(19) United States
(12) Patent Application Publication
(10) Pub. No.: US 2025/0331936 A1
(43) Pub. Date: Oct. 30, 2025

(54) SYSTEMS AND TECHNIQUES FOR MINIMALLY-INVASIVE PROCEDURES

(71) Applicant: W ENDOLUMINAL ROBOTICS LTD, Ariel (IL)

(72) Inventors: Eli SHAPIRA, Kefar Sava (IL); Nadav PAZ, Jerusalem (IL); Ron KAPPEL, Petah Tikva (IL); Tsahi Itshak GRIMBERG, Kefar Sava (IL)

(21) Appl. No.: 18/869,933

(22) PCT Filed: May 23, 2023
(86) PCT No.: PCT/IB2023/055277

§ 371 Date: Nov. 27, 2024

Related U.S. Application Data
(60) Provisional application No. 63/347,119, filed on May 31, 2022.
(60) Provisional application No. 63/445,796, filed on Feb. 15, 2023.

(51) Int. Cl. A61B 34/32; A61B 1/00; A61B 1/267; A61B 34/00; A61B 34/10; A61B 34/30

(52) U.S. Cl. CPC A61B 34/32; A61B 1/00009; A61B 1/2676; A61B 34/10; A61B 34/71; A61B 2034/105; A61B 2034/301; A61B 2034/302

(57) ABSTRACT

A system for use with a mouth and a lung of a subject includes a catheter and a robot. The catheter has a head, and a tube that has a distal steering region. The robot includes a manipulator structure that includes a steering manipulator and an advancement manipulator. The steering manipulator is configured to receive the head in a manner that operatively couples the steering manipulator to the steering region such that a curvature of the steering region is adjustable by the steering manipulator manipulating the head. The advancement manipulator is configured to receive a part of the tube such that operation of the advancement manipulator feeds the tube through the advancement manipulator in a manner that (i) pulls the head and the steering manipulator distally toward the advancement manipulator and the mouth, and (ii) pushes at least the part of the tube distally through the mouth toward the lung.

### CROSS-REFERENCES TO RELATED APPLICATIONS

The present application claims priority to Provisional U.S. Patent Application 63/347,119 to Shapira et al., filed May 31, 2022, and titled “Techniques for accessing lung tissue using a tubular assembly”; and to Provisional U.S. Patent Application 63/445,796 to Shapira et al., filed Feb. 15, 2023, and titled “Control system for endoscopic assembly”.

The present application is also related to PCT/IB2022/057505 to Shapira et al., filed Aug. 11, 2022, titled “Two-pronged approach for bronchoscopy,” which published as WO 2023/017460; and to PCT/IB2022/058307 to Shapira et al., filed Sep. 4, 2022, titled “Steerable tubular assembly for bronchoscopic procedures,” which published as WO 2023/047219.

Each of the above applications is incorporated herein by reference.

### FIELD OF THE INVENTION

The present disclosure relates in general to systems and techniques for medical procedures. More specifically, the present disclosure relates to systems and techniques for manipulation of tubular structures such as catheters for medical procedures such as bronchoscopy.

### BACKGROUND

Steerable tubes, such as catheters, are routinely used to access body cavities of patients. However, configuring such steerable tubes for steering and advancement through particular anatomical lumens, such as airways, remains a challenge. Endoscopic systems may use various means of controlling a steering region of a catheter tube, with varying levels of accuracy in maintaining precise coordinates of a tip of the steering region.

### SUMMARY OF THE INVENTION

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

The present disclosure relates, inter alia, to methods and systems for carrying out an endoscopic procedure on a subject. In some implementations, the procedure may be carried out using two endoscopic catheters, each controlled independently. It is to be noted that the systems and techniques described herein may be applicable to various endoluminal/transluminal procedures including, but not limited to, bronchoscopic, gastroscopic, colonoscopic, and/or transvascular procedures. Nonetheless, the present disclosure focuses on bronchoscopic procedures, for which the systems and techniques described herein may be particularly advantageous.

In some implementations, the system comprises a robot configured to maneuver one or more catheters into and within a lumen of the subject, e.g. the bronchial airways of a lung of the subject. The robot may comprise a manipulator structure guided by a robotic control system. The control system may be configured, e.g. to concurrently guide and maneuver a pair of catheters toward their respective sites.

For some such implementations, a computer model of the lung (which may be generated using imaging data such as CT images and/or MRI images) may be used by the robotic controller to determine the position of the first tube within the airways, e.g. by mapping, onto the computer model, real-time positioning data—e.g. imaging data generated from ultrasound transceiver(s) and/or camera(s) at the end of the tubes, and/or data (e.g. electromechanical data) from sensors on the tubes and/or the robotic controller. The imaging and tool sites are typically present in (e.g. pre-entered into) the computer model, such that the robotic controller can assess whether the tubes are correctly positioned at their respective sites. The designation of the sites and/or the routes may be based on one or more parameters, which are typically parameters of the lung/airways (e.g. derived from the computer model) and/or characteristics of the system to be used (e.g. of the ultrasound transducer and/or the tool). Typically, this designation is performed by circuitry (e.g. running software and/or an algorithm) that uses one or more such parameters as inputs.

Because the imaging and tool sites are typically designated such that the target and the tool will appear in the field of view of the ultrasound transceiver, the imaging and tool sites and/or routes are typically designated as pairs. That is, rather than merely assessing a quality of a given candidate imaging site/route in isolation, or a quality of a given candidate tool site/route in isolation, the designation techniques/algorithms disclosed herein typically assess these sites/routes as candidate pairs—each candidate pair including a candidate imaging site/route and a candidate tool site/route. For example, a candidate pair may only be considered suitable if (i) the target and the tool site of the pair are within the effective imaging range of the imaging site, and (ii) both the imaging site and the tool site are accessible by their respective tubes.

In some implementations, the system may be configured to maneuver the one or more catheters into and within a lumen of the subject, e.g. the bronchial airways of a lung of the subject. The robot may comprise a manipulator structure guided by a control system. The manipulator structure may comprise one or more manipulator assemblies, each manipulator assembly comprising a steering manipulator and an advancement manipulator, e.g. a tube feeder, typically positioned at a distance from each other along an advancement path comprising a track or rail. The control system may comprise a user interface and/or a hand control for interfacing with and manipulating the manipulator structure.

The catheter may be provided as part of the manipulator assembly, or may be, e.g. a consumable component, provided separately. The catheter comprises a head and a tube having an intermediate region and a distal steering region, the steering region controllable by a set of wires, e.g. two, three, or four wires. Each wire is typically connected proximally to a slidable plunger provided as part of the head. The head thus comprises a plunger for each wire, the plungers independently and slidably coupled to a stem of the head. The plungers and the stem may be complementarily shaped to rotationally lock the first and second plungers to the stem. The complementary shapes may define, e.g. a keyed joint, such that the plungers and the stem are rotatable as a unit.

The manipulator structure typically comprises a pair of manipulator assemblies, each assembly configured to receive a catheter. In some implementations, the manipulator structure may comprise three or more manipulator assemblies. In such implementations, one or more manipulator assemblies may be configured to receive and guide a catheter configured to provide a camera, while additional manipulator assemblies may guide catheters carrying biopsy tools or other medical instruments.

The head of the catheter is configured to be inserted into the steering manipulator, and the tube is threadable through, and advanceable by, the advancement manipulator. The catheter thus provides a slidable coupling between the steering manipulator and the advancement manipulator along the advancement path, e.g. a track or rail. That is, the manipulator structure is configured such that the advancement manipulator is repositionable with respect to the steering manipulator.

The control system is configured to provide separate control of the steering manipulator and the advancement manipulator, such that the steering manipulator typically controls rotation, bending, and straightening of the steering region of the tube, whereas the advancement manipulator may control advancement of the catheter, i.e. the steering region thereof. In some implementations, rotation of the catheter may be provided by the advancement manipulator rather than by the steering manipulator. The advancement manipulator is typically situated close to the subject and may be stationary with respect to the rail, such that the tube is fed through the advancement manipulator into and within the lumen of the subject. Feeding the tube through the advancement manipulator draws the head of the catheter within the steering manipulator after the tube, such that the steering manipulator is pulled distally along the rail. The steering manipulator may be configured to slide or retreat passively proximally along the rail when not being actively pulled distally by the advancement manipulator. Such an arrangement of advancement manipulator, catheter, and steering manipulator contributes to straightening of the intermediate region of the tube during active control by the control system, thus facilitating greater accuracy in positioning of the distal steering region.

Control of bending and straightening of the steering region is accomplished by sliding the plungers along the stem of the head. When the catheter head is engaged with the steering manipulator, each plunger sits in a cradle fixedly attached to an abutment within the steering manipulator. The abutment is slidably connected via a spring to an actuator (e.g. a linear actuator) whose linear movement compresses or relaxes the spring and concurrently moves the plunger distally or proximally, resulting in bending or straightening of the wire to which it is attached. Each linear actuator, spring, cradle and abutment comprise an individual control unit. The steering manipulator comprises a control unit for each wire. In a two-wire system, one wire configured to bend the steering region and the other wire configured to straighten the steering region, the steering manipulator comprises two control units.

A pair of encoders or position readers may be provided for each control unit. A first encoder is configured to read a position of the linear actuator, and a second encoder is configured to read a position of the abutment, i.e. the cradle and the plunger removably coupled thereto. The control system is configured to use the position information provided by the encoders to determine (1) a distance moved by the respective plunger, and thus the resulting movement of the connected wire in the steering region; and (2) a force applied to a given spring between an actuator and an abutment/cradle pair of a given control unit, and thus the force applied to the steering region via the wire attached to the associated plunger. Conversely, the control system is configured to respond to a force applied to the steering region via a wire by adjusting the force applied to the respective plunger by the associated linear actuator, thus maintaining a given curvature of the steering region.

The position of the linear actuator is controlled by a motor having a rotational mechanism configured to move the actuator linearly along a threaded rod, whereas the positions of the abutment, cradle, and associated plunger are determined by passive or reactive movement caused by movement of the linear actuator and/or attached spring. By knowing the spring constant of the spring coupled to the linear actuator, and the distance over which the plunger moves, the force applied via the wire to the steering region may be determined. The reverse situation also applies: i.e. if the steering region encounters resistance from the walls of the lumen through which the catheter tube passes, this force may be registered and calculated by the control system.

Rotation of the steering region is accomplished by a motor configured to rotate the entire catheter head within the steering manipulator. Because the plungers are fixed axially but not rotationally within the cradles, the steering manipulator is able to control bending and straightening of the steering region, via linear motion of the plungers, independently of rotation of the catheter head, which simultaneously rotates the tube to which the head is fixedly attached.

Thus, the steering manipulator may comprise three motors, one to move each linear actuator, and a third to rotate the catheter head. In some implementations, the catheter may comprise three plungers, each separately slidable along the stem of the head, and each attached to a separate wire in a manner that allows linear movement of the three plungers along the head to control movement of the steering region. In implementations in which the catheter has three plungers, the corresponding steering manipulator may be configured with three cradles, one for each plunger, and three corresponding control units. In some such implementations, the catheter head would not require rotation; in such cases, the third motor may therefore be the motor for the third wire, rather than for rotation of a catheter having two wires. Thus, for either a two-wire catheter with rotation, or for a three-wire catheter, the head is configured in a manner that allows 360 degrees of motion of the steering region.

Further verification of a position of the distal tip of the tube may be acquired by positioning a sensor at or near the advancement manipulator. The sensor senses the linear advancement of the tube, and/or the rotation of cylindrical components of the advancement manipulator through which the tube moves, thus providing another means of verifying the distance through which the steering region has advanced. The sensor may also be configured to sense rotation of the tube, thus providing (optionally in combination with data collected from the steering manipulator) an indication of the angle and position at which the steering region is disposed.

Visualization of a distal tip of the steering region, e.g. within the airways, may be facilitated by inserting a camera along the tube. Images taken by the camera and provided to the control system may be used with image recognition software or other pattern recognition algorithms to determine the location of the steering region within the airways of the subject.

Any of the techniques, methods, operations, steps, etc. described or suggested herein can be performed on a living animal (e.g., human, other mammal, etc.) or on a non-living simulation, such as a cadaver, a cadaver heart, an anthropomorphic ghost, and/or a simulator device (which may include computerized and/or physical representations of body parts, tissue, etc.).

There is therefore provided, in accordance with some implementations, a system, including a catheter and/or a robot. The catheter may include a head at a proximal region of the catheter, and/or a tube. The tube may have a distal portion configured to be advanced into the subject via a body orifice of the subject, and/or a steering region at the distal portion.

The robot may include a manipulator structure that defines an advancement path and includes a manipulator assembly.

The manipulator assembly may include a steering manipulator and/or an advancement manipulator.

The steering manipulator may be slidable along the advancement path. The steering manipulator may be configured to receive the head in a manner that operatively couples the steering manipulator to the steering region such that a curvature of the steering region is adjustable by the steering manipulator manipulating the head.

The advancement manipulator may be configured to receive the tube such that operation of the advancement manipulator draws the steering manipulator along the advancement path by feeding the tube though the advancement manipulator.

For some implementations, the manipulator assembly is configured such that the advancement manipulator is positionable distally from the steering manipulator to define a separation, between the steering manipulator and the advancement manipulator, of at least 40 cm.

For some implementations, the manipulator assembly is configured such that the steering manipulator is pullable, by the advancement manipulator feeding the tube, to reduce the separation to less than 20 cm.

For some implementations, the manipulator structure is configured such that the operation of the advancement manipulator feeds the tube through the advancement manipulator and through the body orifice into the subject while the advancement manipulator remains stationary with respect to the body orifice.

For some implementations, the manipulator assembly is configured to be loaded with the catheter such that a region of the tube, suspended between the advancement manipulator and the steering manipulator, is substantially unsupported by the manipulator assembly.

For some implementations, the manipulator assembly is configured to be loaded with the catheter such that distally beyond the advancement manipulator the tube is substantially unsupported by the manipulator assembly.

For some implementations, the catheter is sterilized.

For some implementations, the manipulator structure further includes a sensor, the sensor positioned and configured to sense linear advancement of the catheter; and/or provide an advancement output indicative of the sensed linear advancement.

For some implementations, the manipulator structure is configured such that the advancement path is substantially horizontal.

For some implementations, the manipulator structure is configured such that the advancement path is substantially vertical.

For some implementations, the manipulator structure is arranged such that drawing of the steering manipulator along the advancement path by the advancement manipulator draws the steering manipulator closer to the advancement manipulator.

For some implementations, the robot further includes a robotic control system.

For some implementations, the manipulator structure is configured such that the advancement path is substantially straight between the steering manipulator and the advancement manipulator.

For some implementations, the manipulator structure is configured such that the advancement path is curved between the steering manipulator and the advancement manipulator.

For some implementations, the advancement manipulator is configured to rotate the catheter by rotating the tube.

For some implementations, the advancement manipulator is configured to allow rotational slipping of the tube.

For some implementations, the advancement manipulator is configured to disallow rotational slipping of the tube.

For some implementations, the steering manipulator is biased to retreat proximally along the advancement path.

For some implementations, the steering manipulator is biased to retreat proximally along the advancement path by the advancement path sloping downward proximally.

For some implementations, the steering manipulator is biased to retreat proximally along the advancement path by a pulley attached to a slidable mounting.

For some implementations, the steering manipulator is biased to retreat proximally along the advancement path by a spring.

For some implementations, the steering manipulator is biased to retreat proximally along the advancement path by gravitational pull.

For some implementations, the catheter further includes a first wire and a second wire, each of the first wire and the second wire extending from the steering region proximally along the tube.

The head may further include a stem; a first plunger, operatively coupled to the steering region by being attached to the first wire, and mounted on the stem to be slidable linearly along the stem; and/or a second plunger, operatively coupled to the steering region by being attached to the second wire, and mounted on the stem to be slidable linearly along the stem independently of the first plunger.

For some implementations, the steering manipulator is configured to receive the head in a manner that operatively couples the steering manipulator to the first plunger and the second plunger in a manner that configures the steering manipulator to control sliding of the first plunger and the second plunger linearly along the stem.

For some implementations, each of the first plunger and the second plunger is slidably coupled to the stem independently of the other plunger.

For some implementations, application of a sliding force to either plunger slides the plunger in a first direction along the stem in a manner that adjusts a curvature of the steering region by applying tension to the respective wire, and releasing the sliding force allows the respective wire to relax, by the wire responsively pulling the plunger in a reverse direction along the stem.

For some implementations, application of a force to the head adjusts a curvature of the steering region by applying tension to one of the first wire or the second wire, and concurrent tensioning of both wires adjusts a stiffness of the steering region such that the steering region maintains a specific curvature.

For some implementations, the first wire is operatively coupled to the steering region in a manner that causes deflection of the steering region in a first direction upon tensioning of the first wire, and the second wire is operatively coupled to the steering region in a manner that causes deflection of the steering region in a second direction upon tensioning of the second wire, the second direction being opposite to the first direction.

For some implementations, the steering manipulator is configured to control, via sliding of the first plunger and the second plunger linearly along the stem, (i) a curvature of the steering region, and (ii) a stiffness of the steering region.

For some implementations, the system is configured such that loading the head into the steering manipulator enables the steering manipulator to rotate, bend, and straighten the steering region.

For some implementations, for each of the plungers, the plunger is mounted on the stem in a manner in which, while the head is not engaged by the steering manipulator, the plunger is slidable freely along at least part of the stem.

For some implementations, the first plunger is configured to apply tension to the first wire to control a curvature of the steering region.

For some implementations, the second plunger is configured to apply tension to the second wire to control a curvature of the steering region.

For some implementations, the steering manipulator is configured to receive the head in a manner that enables the steering manipulator to, while remaining operatively coupled to the first plunger and the second plunger, rotate the steering region by rotating the head.

For some implementations, the steering manipulator includes a motor configured to rotate the steering region by rotating the head while the head is loaded within the steering manipulator.

For some implementations, the motor is operatively coupled to a drive axle, and/or the head further includes a head gearwheel, coupled to the stem.

The system may further include a single-use gearwheel, configured to be temporarily mounted on the drive axle such that loading the head into the steering manipulator operatively couples the motor to the head gearwheel via the drive axle and the single-use gearwheel, the single-use gearwheel being discardable with the catheter after use.

For some implementations, the catheter is a first catheter, the steering manipulator is a first steering manipulator, and the system further includes a second catheter and a second steering manipulator configured to engage a head of the second catheter in a manner that configures the steering manipulator to manipulate a steering region of the second catheter.

The system may further include a robotic control system, electronically couplable to both the first steering manipulator and the second steering manipulator in a manner that enables the robotic control system to control both the first catheter and the second catheter.

For some implementations, the first plunger and the second plunger and the stem are complementarily shaped to rotationally lock the first plunger and the second plunger to the stem.

For some implementations, the complementary shaping defines a keyed joint.

For some implementations, the stem has a noncircular outer cross-section, and each of the first plunger and the second plunger has a complementary noncircular inner cross-section, thereby rotationally locking the first plunger and the second plunger to the stem.

For some implementations, the steering manipulator includes a first cradle, configured to, upon the steering manipulator receiving the head, cradle the first plunger in a manner that operatively couples the steering manipulator to the first plunger while allowing the first plunger to slip rotationally within the first cradle; and/or a second cradle, configured to, upon the steering manipulator receiving the head, cradle the second plunger in a manner that operatively couples the steering manipulator to the second plunger while allowing the second plunger to slip rotationally within the second cradle.

For some implementations, the system further includes a track on which the steering manipulator is slidably mounted.

For some implementations, the slidable mounting of the steering manipulator on the track enables distal advancement and proximal retraction of the catheter.

For some implementations, the first wire is operatively coupled to the steering region in a force-multiplication arrangement configured to increase a mechanical advantage of the first wire on the steering region.

For some implementations, the second wire is operatively coupled to the steering region in a second force-multiplication arrangement configured to increase a mechanical advantage of the second wire on the steering region.

For some implementations, the manipulator structure includes a mount from which the steering manipulator hangs.

For some implementations, the manipulator structure is configured such that operation of the advancement manipulator draws the steering manipulator downward along the advancement path by feeding the tube though the advancement manipulator.

For some implementations, the manipulator structure includes a winch configured to lift the steering manipulator upwards in retreat along the advancement path.

For some implementations, the system further includes an imaging device, positionable at the distal portion of the catheter; and/or a data-processing system including means for carrying out the steps in paragraph 374.

For some implementations, the robot includes a robotic control system that includes the data-processing system, and that is configured to electronically operate the manipulator structure.

For some implementations, the system further includes an imaging device, positionable at the distal portion of the catheter; and/or a data-processing system including means for carrying out the steps in paragraph 416.

For some implementations, the robot includes a robotic control system that includes the data-processing system, and that is configured to electronically operate the manipulator structure.

For some implementations, the advancement manipulator includes a set of rollers.

For some implementations, the set of rollers is configured to rotate the catheter by rotating the tube.

For some implementations, the set of rollers is configured for single use.

For some implementations, the robot further includes a robotic control system, configured to electronically control the manipulator structure.

For some implementations, the manipulator assembly is a first manipulator assembly, and the manipulator structure includes a second manipulator assembly, the robotic control system configured to electronically coordinate control of the first manipulator assembly and the second manipulator assembly.

For some implementations, the system further includes a third manipulator assembly, and the robotic control system is further configured to electronically coordinate control of the first manipulator assembly, the second manipulator assembly, and the third manipulator assembly.

For some implementations, the head includes a stem, a first plunger, and a second plunger, and/or the steering manipulator is configured to receive the head in a manner that operatively couples the steering manipulator to the steering region such that (i) the steering manipulator sliding the first plunger in a first direction along the stem bends the steering region, and/or (ii) the steering manipulator sliding the second plunger in a first direction along the stem straightens the steering region.

For some implementations, the robotic control system is configured to electronically receive information from the manipulator structure.

For some implementations, the robotic control system is configured to electronically control the manipulator structure responsively to the information.

For some implementations, the manipulator structure includes a track that defines at least part of the advancement path.

For some implementations, the track includes a rail.

For some implementations, the system further includes a mount configured to movably support the advancement manipulator.

For some implementations, the steering manipulator and the advancement manipulator are arranged horizontally with respect to each other.

For some implementations, the steering manipulator and the advancement manipulator are arranged vertically with respect to each other.

For some implementations, the steering manipulator and the advancement manipulator are arranged substantially perpendicular to a craniocaudal axis of a real or simulated subject undergoing a procedure facilitated by the robot.

For some implementations, the system further includes a user interface enabled to facilitate, via the manipulator structure, advancement, steering, and rotation of the steering region.

For some implementations, the user interface includes a hand-held controller.

For some implementations, the user interface includes a display screen.

For some implementations, the system further includes a gate configured to secure the head in the steering manipulator.

For some implementations, the head is removable from the steering manipulator by opening the gate.

For some implementations, the catheter is configured to become limp responsively to the head being disengaged from the steering manipulator.

For some implementations the head includes (i) a stem, (ii) a first plunger, operatively coupled to the steering region via a first wire, and mounted on the stem to be slidable linearly along the stem, and/or (iii) a second plunger, operatively coupled to the steering region via a second wire, and mounted on the stem to be slidable linearly along the stem independently of the first plunger. The steering manipulator may include a first control unit that includes (i) a first actuator, (ii) a first spring, and/or (iii) a first cradle, coupled to the first actuator via the first spring, and configured to receive the first plunger; and/or a second control unit that includes (i) a second actuator, (ii) a second spring, and/or (iii) a second cradle, coupled to the second actuator via the second spring, and configured to receive the second plunger. The steering manipulator may be configured to manipulate the steering region by (i) actuating the first actuator to, via the first spring, slide the first plunger linearly along the stem, and/or (ii) actuating the second actuator to, via the second spring, slide the second plunger linearly along the stem.

For some implementations, the catheter is configured such that linear sliding of the first plunger along the stem bends the steering region.

For some implementations, the catheter is configured such that linear sliding of the second plunger along the stem straightens the steering region.

For some implementations, the steering manipulator further includes a third control unit, each of the first, second, and third control units being configured to pull a respective wire extending from the respective control unit to the steering region.

For some implementations, the steering manipulator includes a housing, and each of the first control unit and the second control unit includes (i) an actuator encoder configured to provide an actuator output indicative of a linear position of the respective actuator with respect to the housing, and/or (ii) a cradle encoder configured to provide a cradle output indicative of a linear position of the respective cradle with respect to the housing.

For some implementations, the actuator encoder is fixedly coupled to the actuator.

For some implementations, the cradle encoder is fixedly coupled to the cradle.

For some implementations, the actuator output is indicative of a linear position of the actuator encoder with respect to the housing.

For some implementations, the cradle output is indicative of a linear position of the cradle encoder with respect to the housing.

For some implementations, the robot further includes a robotic control system configured to use the cradle output and the actuator output to control a force applied to each spring by the respective actuator.

For some implementations, the actuator encoder includes a first readhead paired with a first scale, and the cradle encoder includes a second readhead paired with a second scale.

For some implementations, the first scale and the second scale are fixedly attached to the housing.

For some implementations, the first readhead is fixedly attached to the actuator and the second readhead is fixedly attached to the cradle.

For some implementations, for each of the first control unit and the second control unit, when the corresponding plunger is disposed in the cradle, the control unit is configured such that a distance between the actuator and the cradle is dependent on tension in the corresponding wire.

For some implementations, the system further includes a robotic control system, configured to, for each of the first control unit and the second control unit, receive the actuator output and the cradle output, and determine a magnitude of tension in the corresponding wire responsively to the actuator output and the cradle output.

For some implementations, for each of the first control unit and the second control unit, responsively to receiving the actuator output and the cradle output, the robotic control system is configured to calculate a distance between the actuator and the cradle, and to determine a magnitude of the tension responsively to the calculated distance.

For some implementations, the robotic control system is configured to balance tension on both wires.

For some implementations, the robotic control system is configured to adjust curvature of the steering region while maintaining stiffness on both wires.

For some implementations, for each of the first control unit and the second control unit: the spring has a predetermined spring constant, and/or the robotic control system is configured to determine the magnitude of tension in the corresponding wire responsively to the actuator output, the cradle output, and the predetermined spring constant.

For some implementations, the robotic control system is configured to prevent the magnitude of the tension in the corresponding wire from rising above a predetermined level.

For some implementations, the robotic control system is configured to warn an operator about the magnitude of the tension in the corresponding wire rising above a predetermined level.

For some implementations, concurrent application of a balanced magnitude of tension to each wire at a point at which the steering region has a specific curvature is configured to stiffen the steering region in the specific curvature.

For some implementations, the steering manipulator further includes a first motor configured to produce linear movement of the first actuator, and responsively, the first cradle and the first plunger; and/or a second motor configured to produce linear movement of the second actuator, and responsively, the second cradle and the second plunger.

For some implementations, linear movement of the first plunger bends the steering region.

For some implementations, linear movement of the second plunger straightens the steering region.

For some implementations, rotation of the head rotates the steering region.

For some implementations, the steering manipulator further includes a third motor.

For some implementations, the third motor is configured to rotate the head.

For some implementations, the system further includes a sensor configured to sense forward and rotational movement of the tube with respect to the advancement manipulator.

For some implementations, the sensor includes (i) a rider, the sensor being resiliently connected to the advancement manipulator in a manner that maintains contact between the rider and the tube such that the rider rolls responsively to movement of the tube, and/or (ii) an optical reader mounted facing the rider, and configured to detect rolling of the rider, and/or responsively to detecting the rolling, provide an output indicative of the movement.

For some implementations, rolling of the rider enables verification of movement of the tube by the optical reader in two degrees of motion.

For some implementations, the sensor is connected to the advancement manipulator in a manner that rolling of the rider follows movement of the tube.

For some implementations, the rider is spherical.

For some implementations, the sensor is disposed distally to the advancement manipulator.

For some implementations, the manipulator assembly is a first manipulator assembly, the catheter is a first catheter, and/or the system further includes (i) a second catheter, and/or (ii) a second manipulator assembly, defining a second advancement path, such that the robot is configured to control the first catheter and the second catheter independently and in parallel with each other.

For some implementations, the system further includes (i) a third catheter, and (ii) a third manipulator assembly that defines a third advancement path, such that the robot is configured to control the first catheter, the second catheter, and the third catheter independently and in parallel with each other.

For some implementations, the manipulator structure is configured such that the advancement manipulator is repositionable with respect to the steering manipulator.

For some implementations, the manipulator structure is configured such that the advancement manipulator is movable with respect to the steering manipulator in a manner that facilitates positioning of the advancement manipulator adjacent to the body orifice of the subject.

For some implementations, the manipulator structure is configured such that the advancement manipulator is movable with respect to the steering manipulator in a manner that facilitates positioning of the advancement manipulator adjacent to the body orifice of the subject.

There is further provided, in accordance with some implementations, a system, including a catheter and/or a steering manipulator. The catheter may include a tube, a first wire and a second wire, and/or a head. The tube may have a steering region at a distal portion thereof. The first and second wires may each extend from the steering region proximally along the tube.

The head may include (i) a stem, (ii) a first plunger, operatively coupled to the steering region by being attached to the first wire, and mounted on the stem to be slidable linearly along the stem, and/or (ii) a second plunger, operatively coupled to the steering region by being attached to the second wire, and mounted on the stem to be slidable linearly along the stem independently of the first plunger.

The steering manipulator may be configured to engage the head by receiving the head in a manner that operatively couples the steering manipulator to the first plunger and the second plunger in a manner that configures the steering manipulator to manipulate the steering region by controlling sliding of the first plunger and the second plunger linearly along the stem.

For some implementations, the catheter is sterilized.

For some implementations, the steering manipulator is configured to stiffen the steering region by facilitating balancing of tension between the first wire and the second wire.

For some implementations, the catheter is configured such that, while the steering region has a given curvature, balanced tension applied to the first wire and the second wire stiffens the steering region in the given curvature.

For some implementations, the first wire is operatively coupled to the steering region in a manner that causes deflection of the steering region in a first direction upon pulling of the first wire, and the second wire is operatively coupled to the steering region in a manner that causes deflection of the steering region in a second direction upon pulling of the second wire, the second direction being opposite to the first direction.

For some implementations, the steering manipulator is configured to control, via sliding of the first plunger and the second plunger linearly along the stem, (i) a curvature of the steering region, and (ii) a stiffness of the steering region.

For some implementations, the system is configured such that engaging the head with the steering manipulator enables the steering manipulator to rotate, bend, and straighten the steering region.

For some implementations, for each of the first plunger and the second plunger, the plunger is mounted on the stem in a manner in which, while the head is not engaged by the steering manipulator, the plunger is slidable freely along at least part of the stem.

For some implementations, the first plunger is configured to tension the first wire to control a curvature of the steering region.

For some implementations, the second plunger is configured to tension the second wire to control a curvature of the steering region.

For some implementations, the steering manipulator includes a motor enabled to rotate the catheter while the head is engaged with the steering manipulator.

For some implementations, the steering manipulator is configured to receive the head in a manner that enables the steering manipulator to, while remaining operatively coupled to the first plunger and the second plunger, rotate the steering region by rotating the head.

For some implementations, the system is configured such that engagement of the head by the steering manipulator enables measurement and calibration of tension on each of the first wire and the second wire of the catheter.

For some implementations, the catheter is configured such that, upon the head becoming released from the steering manipulator, the steering region responsively becomes limp.

For some implementations, the system further includes a robotic control system in electronic communication with the steering manipulator, and configured to identify the catheter upon the head being engaged by the steering manipulator.

For some implementations, the robotic control system is configured to, responsively to identifying the catheter, set one or more parameters for manipulation of the catheter.

For some implementations, the one or more parameters include allowable ranges of sliding of the first plunger and the second plunger linearly along the stem.

For some implementations, the one or more parameters include a maximum allowable force with which the steering manipulator may slide the first plunger linearly along the stem.

For some implementations, the one or more parameters include a maximum allowable force with which the steering manipulator may slide the second plunger linearly along the stem.

For some implementations, the steering manipulator includes (1) a first control unit, including a first actuator, a first force sensor, and/or a first cradle, and (2) a second control unit, including a second actuator, a second force sensor, and/or a second cradle. The first cradle may be coupled to the first actuator via the first force sensor, and configured to receive the first plunger. The second cradle may be coupled to the second actuator via the second force sensor, and configured to receive the second plunger. The steering manipulator may be configured to manipulate the steering region by (1) actuating the first actuator to, via the first force sensor, slide the first plunger linearly along the stem, and/or (2) actuating the second actuator to, via the second force sensor, slide the second plunger linearly along the stem.

For some implementations, the first actuator is a first linear actuator, and the second actuator is a second linear actuator.

For some implementations, the steering manipulator includes (1) a first control unit, including a first actuator, a first spring, and/or a first cradle, and (2) a second control unit, including a second actuator, a second spring, and/or a second cradle. The first cradle may be coupled to the first actuator via the first spring, and configured to receive the first plunger. The second cradle may be coupled to the second actuator via the second spring, and configured to receive the second plunger. The steering manipulator may be configured to manipulate the steering region by (1) actuating the first actuator to, via the first spring, slide the first plunger linearly along the stem, and/or (2) actuating the second actuator to, via the second spring, slide the second plunger linearly along the stem.

For some implementations, the first actuator is a first linear actuator, and the second actuator is a second linear actuator.

For some implementations, each of the first control unit and the second control unit is configured such that, when the plunger is disposed in the cradle, a linear distance between the actuator and the cradle is relative to tension in the corresponding wire.

For some implementations, while the head is engaged by the steering manipulator, for each of the first plunger and the second plunger, the system is configured to maintain an axial position of the plunger with respect to the stem by automatically adjusting a force applied to the plunger by the actuator via the spring and the cradle.

For some implementations, the catheter is configured such that linear movement of the first plunger along the stem bends the steering region.

For some implementations, the catheter is configured such that linear movement of the second plunger along the stem straightens the steering region.

For some implementations, the steering manipulator includes a motor enabled to rotate the catheter by rotating the head while the head is engaged with the steering manipulator.

For some implementations, for each of the first plunger and the second plunger, while the head is engaged by the steering manipulator with the plunger disposed within the cradle, the steering manipulator is enabled to manipulate the steering region and rotate the head independently and concurrently.

For some implementations, each plunger is rotationally locked with respect to the stem.

For some implementations, each plunger has a circular outer cross-section, allowing it to slip rotationally within its cradle.

For some implementations, the steering manipulator further includes a first motor configured to produce linear movement of the first actuator, and responsively, the first cradle and the first plunger; and/or a second motor configured to produce linear movement of the second actuator, and responsively, the second cradle and the second plunger.

For some implementations, the steering manipulator further includes a third motor.

For some implementations, the third motor is configured to rotate the head.

For some implementations, each of the first control unit and the second control unit includes a cradle encoder configured to provide a cradle output indicative of a linear position of the cradle within the steering manipulator.

For some implementations, the cradle encoder is fixedly coupled to the cradle.

For some implementations, the cradle output is indicative of a linear position of the cradle encoder with respect to the steering manipulator.

For some implementations, each of the first control unit and the second control unit includes an actuator encoder configured to provide an actuator output indicative of a linear position of the respective actuator within the steering manipulator.

For some implementations, the actuator encoder is fixedly coupled to the respective actuator.

For some implementations, the actuator output is indicative of a linear position of the actuator encoder with respect to the steering manipulator.

For some implementations, each of the first control unit and the second control unit includes a force sensor configured to provide a force output indicative of strain on the spring resulting from actuation of the corresponding actuator.

For some implementations, the force sensor includes a strain gauge.

For some implementations, the force sensor is disposed between the cradle and the actuator.

For some implementations, each of the first control unit and the second control unit includes a force sensor configured to provide a force output indicative of force exerted on the cradle by the corresponding actuator via the corresponding spring.

For some implementations, each of the first control unit and the second control unit includes another encoder, configured to provide another output, and/or the system further includes a robotic control system configured to, for each of the first control unit and the second control unit: (i) receive the cradle output and the other output, and/or (ii) responsively to the cradle output and the other output, determine a tension magnitude in the corresponding wire.

For some implementations, for each of the first control unit and the second control unit: the other encoder is an actuator encoder, the other output is an actuator output indicative of a linear position of the actuator within the steering manipulator, and/or the actuator encoder is configured to provide the actuator output.

For some implementations, for each of the first control unit and the second control unit: the other output is indicative of a linear position of the actuator with respect to the cradle, and/or the other encoder is configured to provide the other output that is indicative of the linear position of the actuator with respect to the cradle.

For some implementations, for each of the first control unit and the second control unit, responsively to receiving the cradle output and the other output, the control system is configured to calculate a distance between the actuator and the cradle, and to determine the tension magnitude in the corresponding wire responsively to the calculated distance.

For some implementations, for each of the first control unit and the second control unit: the spring has a predetermined spring constant, the other output is the actuator output, and/or the control system is configured to determine the tension magnitude in the corresponding wire responsively to the actuator output, the cradle output, and the predetermined spring constant.

For some implementations, for each of the first control unit and the second control unit: the other output is a force sensor output, and/or the robotic control system is configured to determine the tension magnitude in the corresponding wire responsively to the force sensor output.

For some implementations, the robotic control system is configured to disallow the steering manipulator from increasing the tension magnitude in the corresponding wire to above a predetermined level.

For some implementations, the robotic control system is configured to warn an operator upon the tension magnitude in the corresponding wire rising above a predetermined level.

For some implementations, the robotic control system is configured to stiffen the steering region by balancing the tension magnitude of the first wire with the tension magnitude of the second wire.

For some implementations, the catheter is configured such that, while the steering region has a given curvature, balanced tension applied to each wire stiffens the steering region in the given curvature.

For some implementations, the actuator encoder includes a first readhead paired with a first scale, and the cradle encoder includes a second readhead paired with a second scale.

For some implementations, the first scale is fixedly attached to the steering manipulator.

For some implementations, the second scale is fixedly attached to the steering manipulator.

For some implementations, the first readhead is fixedly attached to the corresponding actuator.

For some implementations, the second readhead is fixedly attached to the cradle.

For some implementations, each of the first control unit and the second control unit includes an actuator encoder configured to provide an actuator output indicative of a linear position of the actuator within the steering manipulator.

For some implementations, the actuator encoder is fixedly coupled to the actuator.

For some implementations, the actuator output is indicative of a linear position of the actuator encoder with respect to the steering manipulator.

For some implementations, each of the first control unit and the second control unit includes a force sensor configured to provide a force output indicative of strain on the spring resulting from actuation of the corresponding actuator.

For some implementations, the force sensor includes a strain gauge.

For some implementations, the force sensor is disposed between the cradle and the actuator.

For some implementations, each of the first control unit and the second control unit includes a force sensor configured to provide a force output indicative of force exerted on the cradle by the corresponding actuator via the corresponding spring.

For some implementations, each of the first control unit and the second control unit is configured to provide an output indicative of a linear distance between the cradle and the actuator.

For some implementations, for each of the first control unit and the second control unit, the control unit includes a movable linear scale, fixed with respect to the actuator, and the control unit is configured to provide the output responsively to a linear position of the cradle with respect to the movable linear scale.

For some implementations, for each of the first control unit and the second control unit, the control unit includes a movable linear scale, fixed with respect to the cradle, and the control unit is configured to provide the output responsively to a linear position of the actuator with respect to the movable linear scale.

For some implementations, each of the first control unit and the second control unit includes a first encoder and a second encoder.

For some implementations, for each of the first control unit and the second control unit: the first encoder is configured to provide an output indicative of a position of the cradle within the steering manipulator, and/or the second encoder is configured to provide an output indicative of a linear distance between the cradle and the actuator.

For some implementations, for each of the first control unit and the second control unit: the first encoder is configured to provide an output indicative of a position of the actuator within the steering manipulator, and/or the second encoder is configured to provide an output indicative of a linear distance between the cradle and the actuator.

For some implementations, for each of the first control unit and the second control unit: the first encoder is configured to provide an output indicative of a position of the cradle within the steering manipulator, and/or the second encoder is configured to provide an output indicative of a position of the actuator within the steering manipulator.

For some implementations, the system further includes a control system, each of the first encoder and the second encoder is configured to provide a respective output, and/or the control system is configured to receive the respective output of each of the first encoder and the second encoder.

For some implementations, the first wire is operatively coupled to the steering region in a pulley arrangement configured to increase a mechanical advantage of the first wire on the steering region.

For some implementations, the second wire is operatively coupled to the steering region in a second pulley arrangement configured to increase a mechanical advantage of the second wire on the steering region.

For some implementations, the system further includes a track on which the steering manipulator is slidably mounted.

For some implementations, the slidable mounting of the steering manipulator on the track enables distal advancement and proximal retreat of the steering manipulator.

For some implementations, the steering manipulator is biased to retreat proximally along the track.

For some implementations, the steering manipulator is biased to retreat proximally along the track by the track sloping downward proximally.

For some implementations, the steering manipulator is biased to retreat proximally along the track by a pulley attached to the slidable mounting.

For some implementations, the steering manipulator is biased to retreat proximally along the track by a spring.

For some implementations, the steering manipulator is biased to retreat proximally along the track by gravitational pull.

For some implementations, the system further includes a counterweight, coupled to the steering manipulator in a manner that provides the gravitational pull.

For some implementations, the track is sloped in a manner that provides the gravitational pull.

For some implementations, the first plunger and the second plunger and the stem are complementarily shaped to rotationally lock the first plunger and the second plunger to the stem.

For some implementations, the complementary shapes define a keyed joint.

For some implementations, the stem has a noncircular outer cross-section, and each of the first plunger and the second plunger has a complementary noncircular inner cross-section, thereby rotationally locking the first plunger and the second plunger to the stem.

For some implementations, the steering manipulator includes: a first cradle, configured to, upon the steering manipulator receiving the head, cradle the first plunger in a manner that operatively couples the steering manipulator to the first plunger while allowing the first plunger to slip rotationally within the first cradle, and/or a second cradle, configured to, upon the steering manipulator receiving the head, cradle the second plunger in a manner that operatively couples the steering manipulator to the second plunger while allowing the second plunger to slip rotationally within the second cradle.

For some implementations, the steering manipulator is configured to rotate the head and to cause the rotational slipping of the first plunger and the second plunger.

For some implementations: the catheter is a first catheter, the steering manipulator is a first steering manipulator, and/or the system further includes a second catheter and a second steering manipulator configured to engage a head of the second catheter in a manner that configures the steering manipulator to manipulate a steering region of the second catheter.

For some implementations, the system further includes a robotic control system, electronically couplable to both the first steering manipulator and the second steering manipulator in a manner that enables the robotic control system to coordinate control of both the first catheter and the second catheter.

For some implementations: the system further includes a third catheter, and a third steering manipulator, and/or the robotic control system is electronically couplable to the first steering manipulator, the second steering manipulator, and the third steering manipulator in a manner that enables the robotic control system to coordinate control of the first catheter, the second catheter, and the third catheter.

For some implementations, the system further includes a gate configured to maintain engagement of the head by the steering manipulator by securing the head within the steering manipulator.

For some implementations, the head defines a circular surface, and the gate includes a roller configured to ride on the circular surface, facilitating rotation of the catheter while the gate maintains the head secured within the steering manipulator.

There is further provided, in accordance with some implementations, apparatus, including a catheter that includes a tube, a first wire and a second wire, and/or a head. The tube may have a steering region at a distal portion thereof. Each of the first wire and the second wire may extend from the steering region proximally along the tube. The head may include (i) a stem, (ii) a first plunger, mounted on the stem, and operatively coupled to the steering region via the first wire, such that linear sliding of the first plunger along the stem deflects the steering region in a first direction by pulling on the first wire, and/or (iii) a second plunger, mounted on the stem, and operatively coupled to the steering region via the second wire, such that linear sliding of the second plunger along the stem deflects the steering region in a second direction by pulling on the second wire.

For some implementations, the catheter is sterilized.

For some implementations, the head is absent of means to maintain a linear position, along the stem, of either of the first plunger and the second plunger.

For some implementations, the head further includes a head-gearwheel, fixed to the stem.

For some implementations, the first plunger and the second plunger are linearly slidable along the stem independently of each other.

For some implementations, the stem extends through the first plunger and the second plunger.

For some implementations, the apparatus further includes a holder, removably mounted on the head in a manner that inhibits linear sliding of the first plunger along the stem.

For some implementations, the holder is shaped to define a handle that facilitates handling of the head by a human operator.

For some implementations, the manner in which the holder is mounted on the head inhibits linear sliding of the second plunger along the stem.

For some implementations, the manner in which the holder is mounted on the head retains the first plunger at a first-plunger linear position along the stem, and retains the second plunger at a second-plunger linear position along the stem.

For some implementations, the apparatus further includes an electronically-controlled steering manipulator, the head being loadable into the steering manipulator in a manner that operatively couples the steering manipulator to the steering region such that the steering region is adjustable via sliding, by the steering manipulator, of the first plunger and the second plunger linearly along the stem.

For some implementations, the steering manipulator includes a motor, the head further includes a head-gearwheel, fixed to the stem, and/or the manner in which the head is loadable into the steering manipulator operatively couples the head-gearwheel to the motor such that the catheter is rotatable by the motor driving the head-gearwheel.

For some implementations, the motor is operatively coupled to a drive axle, and/or the apparatus further includes a single-use gearwheel, configured to be temporarily mounted on the drive axle such that loading the head into the steering manipulator operatively couples the motor to the head-gearwheel via the drive axle and the single-use gearwheel, the single-use gearwheel being discardable with the catheter after use.

For some implementations, the steering manipulator defines a first push-face with which the steering manipulator is configured to push the first plunger linearly along the stem, and a second push-face with which the steering manipulator is configured to push the second plunger linearly along the stem.

For some implementations, the steering manipulator is configured to push the first plunger proximally along the stem by pushing the first push-face proximally against the first plunger.

For some implementations, the steering manipulator is configured to push the second plunger proximally along the stem by pushing the second push-face proximally against the second plunger.

For some implementations, the apparatus further includes a holder, removably mounted on the head in a manner that maintains the first plunger at a first-plunger linear position along the stem, and maintains the second plunger at a second-plunger linear position along the stem, such that, upon loading of the head into the steering manipulator, the first plunger is positioned at the first push-face, and the second plunger is positioned at the second push-face.

For some implementations, the holder is shaped to define a handle that facilitates loading of the head into the steering manipulator by a human operator.

For some implementations, the holder is removably mounted on the head by snap-fit.

For some implementations, the catheter includes a roller assembly, mounted on the tube, and including a roller configured to feed the tube through the roller assembly.

For some implementations, the apparatus includes a sterile package containing the catheter.

For some implementations, the head further includes a head-gearwheel, fixed to the stem, and the sterile package further contains at least one manipulator-gearwheel that is complementary to the head-gearwheel.

For some implementations, the roller is a roller of a set of rollers, the set of rollers being configured to feed the tube through the roller assembly.

For some implementations, the roller is configured to be pressed into engagement with the tube.

For some implementations, the roller assembly has a rest state in which the roller is disengaged from the tube.

For some implementations, the apparatus further includes an electronically-controlled advancement manipulator, and the roller assembly is loadable onto the advancement manipulator in a manner that configures the advancement manipulator to feed the tube through the roller assembly by rotating the roller.

For some implementations, the advancement manipulator is configured to press the roller into engagement with the tube.

For some implementations, the advancement manipulator is configured to press the roller into engagement with the tube upon loading of the roller assembly onto the advancement manipulator.

For some implementations, the advancement manipulator includes an axle, and the roller assembly is loadable onto the advancement manipulator in a manner that mounts the roller on the axle such that the advancement manipulator becomes configured to feed the tube through the roller assembly by rotating the roller via rotating the axle.

For some implementations, the roller assembly is configured such that the mounting of the roller onto the axle presses the roller into engagement with the tube.

There is further provided, in accordance with some implementations, apparatus including a catheter. The catheter may include a tube, a head, and a feeder. The tube may have a steering region at a distal portion thereof. The head may be at a proximal region of the tube, and/or may be operatively coupled to the steering region such that operation of the head controls bending of the steering region. The feeder may be mounted on the tube, and/or may be configured to feed the tube through the feeder.

For some implementations, the catheter is sterilized.

For some implementations, the apparatus includes a sterile package containing the catheter.

For some implementations, the apparatus is for use with an advancement manipulator, the feeder being loadable onto the advancement manipulator in a manner that configures the advancement manipulator to drive the feeder to feed the tube through the feeder.

For some implementations, the feeder is passive, and is configured to be driven to feed the tube through the feeder.

For some implementations, the feeder includes a roller configured to feed the tube through the feeder.

For some implementations, the feeder has a rest state in which the feeder is mounted on the tube and the roller is disengaged from the tube.

For some implementations, the roller is configured to be pressed into engagement with the tube.

For some implementations, the roller is a feed roller, and the feeder further includes a rider roller that has a rider-roller axis, and that is mounted in the feeder such that feeding of the tube through the feeder by the feed roller rotates the rider roller about the rider-roller axis.

For some implementations, the feeder is configured to feed the tube through the feeder via rotation of the feed roller about a feed-roller axis, and the feed roller is mounted in the feeder such that rotation of the tube within the feeder translates the feed roller along the feed-roller axis.

For some implementations, the feed roller is a first feed roller, the feed-roller axis being a first feed-roller axis, and the feeder includes a second feed roller, complementary to the first feed roller. For some implementations, the feeder is configured to feed the tube through the feeder via concurrent rotation (i) of the first feed roller in a first rotational direction about the first feed-roller axis, and (ii) of the second feed roller in a second rotational direction about a second feed-roller axis, the second rotational direction being opposite to the first rotational direction. For some implementations, the first feed roller and the second feed roller are mounted in the feeder such that rotation of the tube within the feeder concurrently translates (i) the first feed roller in a first axial direction along the first feed-roller axis, and (ii) the second feed roller in a second axial direction along the second feed-roller axis, the second axial direction being opposite to the first axial direction.

For some implementations, the rider roller is mounted in the feeder such that rotation of the tube within the feeder translates the rider roller along the rider-roller axis.

For some implementations, the rider roller is a first rider roller, the rider-roller axis being a first rider-roller axis, and the feeder includes a second rider roller, complementary to the first rider roller. For some implementations, the first rider roller and the second rider roller are mounted in the feeder such that feeding of the tube through the feeder by the feed roller concurrently rotates (i) the first rider roller in a first rotational direction about the first rider-roller axis, and (ii) the second rider roller in a second rotational direction about a second rider-roller axis, the second rotational direction being opposite to the first rotational direction. For some implementations, the first rider roller and the second rider roller are mounted in the feeder such that rotation of the tube within the feeder concurrently translates (i) the first rider roller in a first axial direction along the first rider-roller axis, and (ii) the second rider roller in a second axial direction along the second rider-roller axis, the second axial direction being opposite to the first axial direction.

For some implementations, the apparatus further includes an electronically-controlled advancement manipulator, the feeder being loadable onto the advancement manipulator in a manner that configures the advancement manipulator to feed the tube through the feeder by rotating the feed roller.

For some implementations, the advancement manipulator includes a sensor, and, when the feeder is loaded onto the advancement manipulator, the advancement manipulator is configured to (i) detect the rotation of the rider roller about the rider-roller axis, (ii) responsively to the detected rotation, provide an advancement output indicative of advancement of the tube through the feeder, (iii) detect the translation of the rider roller along the rider-roller axis, and/or (iv) responsively to the detected translation, provide a rotation output indicative of rotation of the tube within the feeder.

For some implementations, the advancement manipulator includes a motorized feed axle; and/or a passive rider axle. For some implementations, the feeder is configured such that loading the feeder onto the advancement manipulator: (i) loads the feed roller onto the feed axle such that the feed roller becomes rotationally locked to the feed axle, but axially slidable along the feed axle, and/or (ii) loads the rider roller onto the rider axle such that the rider roller becomes rotationally and axially locked to the rider axle.

For some implementations, the feed axle and the rider axle are spring-loaded such that, when the feeder is loaded onto the advancement manipulator, the feed axle presses the feed roller into engagement with the tube and the rider axle presses the rider roller into engagement with the tube.

There is further provided, in accordance with some implementations, apparatus for use with an elongate member, the apparatus including an advancement unit that includes a feed axle that lies on a feed-axle axis, a rider axle that lies on a rider-axle axis, a motor, and/or one or more sensors. The motor may be operatively coupled to the feed axle such that operation of the motor rotates the feed axle about the feed-axle axis. The one or more sensors may be operatively coupled to the rider axle in a manner that detects (i) rotation of the rider axle about the rider-axle axis, and/or (ii) translation of the rider axle along the rider-axle axis. The advancement unit may be configured to receive the elongate member such that (i) operation of the motor feeds the elongate member through the advancement unit via rotation of the feed axle about the feed-axle axis in a manner in which the elongate member rotates the rider axle about the rider-axle axis, and/or (ii) rotation of the elongate member within the advancement unit translates the rider axle along the rider-axle axis.

For some implementations, the apparatus further includes a feed roller mounted on the feed axle, and configured to engage the elongate member such that operation of the motor feeds the elongate member through the advancement unit by rotating the feed roller about the feed-axle axis.

For some implementations, the advancement unit is configured to receive the elongate member such that rotation of the elongate member within the advancement unit slides the feed roller along the feed axle.

For some implementations, the apparatus further includes a rider roller mounted on the rider axle, and configured to engage the elongate member such that feeding of the elongate member through the advancement unit rotates the rider axle about the rider-axle axis by rotating the rider roller about the rider-axle axis.

For some implementations, the advancement unit is configured to receive the elongate member such that rotation of the elongate member within the advancement unit translates the rider axle along the rider-axle axis by translating the rider roller along the rider-axle axis.

There is further provided, in accordance with some implementations, apparatus including a catheter for use in operative procedures, the catheter including: a tube having a steering region at a distal portion thereof; a set of wires, extending proximally along the tube from the steering region; and/or a head, attached to a proximal portion of the tube, and operatively coupled to the steering region via the set of wires such that (i) curvature of the steering region is controllable by manipulating the head to apply tension to the set of wires, and/or (ii) the head is configured to not maintain the tension in the wires of the set in an absence of an exogenous force applied to the head.

For some implementations, the catheter is sterilized.

For some implementations, the wires of the set are tensionable independently of each other.

For some implementations, the set of wires includes exactly three wires distributed circumferentially around the tube and extending proximally along the tube.

For some implementations, the set of wires includes exactly two wires extending, opposite each other, proximally along the tube.

For some implementations, the catheter is a single-use catheter.

For some implementations, the catheter is configured such that increasing tension on all of the wires of the set stiffens the steering region.

For some implementations, the steering region of the tube includes intercalating vertebrae.

For some implementations, the stiffening of the steering region is facilitated by the increasing of the tension on all of the wires axially compressing the intercalating vertebrae.

For some implementations, the vertebrae are configured such that, when the steering region is stiffened, the vertebrae slip against each other in a single direction.

For some implementations, the vertebrae are configured such that, when the steering region is stiffened, the vertebrae slip against each other in any direction.

For some implementations, the set of wires includes: a bending wire, tensioning of the bending wire bending the steering region, and/or a straightening wire, tensioning of the bending wire straightening the steering region. The head may include: a stem, fixed to the proximal portion of the tube; a bending plunger attached to the bending wire, and slidable along the stem in a manner that applies tension to the bending wire; and/or a straightening plunger attached to the straightening wire, and slidable along the stem in a manner that applies tension to the straightening wire.

For some implementations, within the steering region, the bending wire is arranged in a force-multiplication arrangement that provides the bending wire with a mechanical advantage.

For some implementations, within the steering region, the mechanical advantage of the bending wire is greater than a mechanical advantage of the straightening wire.

For some implementations, within the steering region, the straightening wire is not arranged in a force-multiplication arrangement.

For some implementations, the mechanical advantage is at least two.

For some implementations, the mechanical advantage is at least three.

For some implementations, the force-multiplication arrangement is a pulley system.

For some implementations, the apparatus further includes a steering manipulator, reversibly engageable with the head, and configured to apply and maintain the exogenous force on the head.

For some implementations, the apparatus further includes a control system configured to electronically control the steering manipulator to apply and maintain the exogenous force on the head.

There is further provided, in accordance with some implementations, apparatus, including a catheter for use in operative procedures, the catheter including a tube, a wire, and/or a head.

The tube may have a steering region at a distal portion thereof. The wire may extend from the steering region proximally along the tube. The head may be attached to a proximal portion of the tube. The head may include a stem, and/or a plunger, slidably coupled to the stem. The head may be connected to the wire such that (i) application of a sliding force to the plunger slides the plunger in a first direction along the stem in a manner that increases a curvature of the steering region by pulling on the wire, and/or (ii) releasing the sliding force allows the wire to responsively pull the plunger in a reverse direction along the stem.

For some implementations, the catheter is sterilized.

For some implementations, the stem is elongate and has a linear axis.

For some implementations the plunger is a first plunger, and the head further includes a second plunger slidably coupled to the stem; the wire is a first wire, and the catheter further includes a second wire, extending from the steering region proximally along the tube, and connected to the second plunger, such that application of a sliding force to the second plunger slides the second plunger in the first direction along the stem in a manner that decreases a curvature of the steering region by applying tension to the second wire.

For some implementations, the first plunger is disposed on the stem axially between the second plunger and the tube.

For some implementations, the steering region of the tube is stiffenable by balanced tensioning of the first wire and the second wire.

For some implementations, the apparatus further includes a steering manipulator, reversibly engageable with the head, and configured to tension the first wire and the second wire independently of each other.

There is further provided, in accordance with some implementations, apparatus, including a catheter for use in operative procedures, the catheter including a flexible tube, a first wire and a second wire, and a head. The flexible tube may have a proximal region, an intermediate region, and a steering region distal to the intermediate region. Each of the first wire and the second wire may extend from the steering region proximally along the tube. The head may be attached to the proximal portion of the tube, to the first wire, and to the second wire, and may be configured (i) to facilitate bending of the steering region by pulling on the first wire, and/or (ii) to facilitate stiffening of the steering region via application of tension to the first wire and the second wire concurrently.

For some implementations, the catheter is sterilized.

For some implementations, the intermediate region is more flexible than the steering region.

For some implementations, the steering region includes a series of vertebrae having a predetermined compressive strength.

For some implementations, the vertebrae are configured to lock in a fixed curvature responsively to the application of the tension to the first wire and the second wire concurrently.

For some implementations, the head includes: a stem; a first plunger, connected to the first wire, and slidably coupled to the stem; and/or a second plunger, connected to the second wire, and slidably coupled to the stem independently of the first plunger.

For some implementations, the head is configured such that, for each of the first plunger and the second plunger, application of a linear force to the plunger pulls on the corresponding wire.

For some implementations, the apparatus further includes a steering manipulator, reversibly engageable with the head, and configured to apply linear force to the first plunger and to apply linear force to the second plunger.

For some implementations, the apparatus further includes a control system configured to electronically control the steering manipulator to apply linear force to the first plunger and to apply linear force to the second plunger.

For some implementations, the control system is configured to electronically control the steering manipulator to stiffen the steering region by applying tension to the first wire and the second wire concurrently.

For some implementations, within the steering region, the first wire is arranged to have a greater mechanical advantage than in the intermediate region.

For some implementations, within the steering region, the greater mechanical advantage is provided by the first wire being arranged in a pulley arrangement.

For some implementations, within the steering region, the second wire is arranged to have a greater mechanical advantage than in the intermediate region.

There is further provided, in accordance with some implementations, a method, including advancing a steering region at a distal portion of a tube of a catheter into a real or simulated subject, the catheter including a first wire and a second wire extending from the steering region proximally along the tube; adjusting a curvature of the steering region by pulling the first wire proximally relative to the tube and to the second wire; and/or subsequently, stabilizing the curvature by balancing tension between the first wire and the second wire.

For some implementations, the steps of adjusting the curvature of the steering region and advancing the steering region are performed concurrently.

For some implementations, the steps of adjusting the curvature of the steering region and stabilizing the curvature of the steering region are performed concurrently.

For some implementations, adjusting the curvature includes pulling the first wire.

For some implementations, stabilizing the curvature includes pulling the second wire while maintaining the steering region in a desired curvature.

For some implementations, advancing the steering region into the subject includes transluminally advancing the steering region into the subject.

For some implementations, transluminally advancing the steering region into the subject includes transbronchially advancing the steering region into the subject.

For some implementations, the method further includes sliding a medical tool within a lumen of the tube while the curvature of the steering region remains stabilized.

For some implementations, sliding the medical tool within the lumen includes advancing the medical tool distally through the lumen.

For some implementations, sliding the medical tool within the lumen includes retracting the medical tool proximally through the lumen.

For some implementations, sliding the medical tool within the lumen includes advancing a camera through the lumen.

For some implementations, sliding the medical tool within the lumen includes advancing a needle through the lumen.

For some implementations, sliding the medical tool within the lumen includes advancing an ultrasound probe through the lumen.

For some implementations, advancing the steering region into the subject is robotically performed by an advancement manipulator.

For some implementations, adjusting the curvature of the steering region and stabilizing the curvature are robotically performed by a steering manipulator.

For some implementations, the catheter is a first catheter, and the method further includes: advancing a steering region at a distal portion of a tube of a second catheter into the subject, the second catheter including a first wire and a second wire extending from the steering region proximally along the tube; adjusting a curvature of the steering region of the second catheter by pulling the first wire of the second catheter proximally relative to the tube of the second catheter and to the second wire of the second catheter; and/or subsequently, stabilizing the curvature of the steering region of the second catheter by balancing tension between the first wire and the second wire of the second catheter.

For some implementations, advancing the steering region of the second catheter into the subject includes advancing the steering region of the second catheter into the subject while the steering region of the first catheter remains within the subject.

For some implementations, adjusting the curvature of the steering region of the second catheter includes adjusting the curvature of the steering region of the second catheter while the steering region of the first catheter remains within the subject.

There is further provided, in accordance with some implementations, a method, including: advancing a steering region at a distal portion of a tube of a catheter into a real or simulated subject, the catheter including a first wire and a second wire extending from the steering region proximally along the tube; concurrently tensioning the first wire and the second wire; and/or adjusting a curvature of the steering region within the subject.

For some implementations, adjusting a curvature of the steering region includes adjusting a curvature of the steering region while maintaining the concurrent tensioning on the first wire and the second wire.

For some implementations, advancing the steering region into the subject includes transbronchially advancing the steering region into the subject.

For some implementations, the catheter is a first catheter, and the method further includes: advancing a steering region at a distal portion of a tube of a second catheter into the subject, the second catheter including a first wire and a second wire extending from the steering region proximally along the tube; concurrently tensioning the first wire and the second wire of the second catheter; and/or adjusting a curvature of the steering region of the second catheter within the subject.

For some implementations, advancing the steering region of the second catheter into the subject includes advancing the steering region of the second catheter into the subject while the steering region of the first catheter remains within the subject.

For some implementations, adjusting the curvature of the steering region of the second catheter includes adjusting the curvature of the steering region of the second catheter while the steering region of the first catheter remains within the subject.

For some implementations: the first wire is arranged within the steering region to define a force-multiplication arrangement, and/or adjusting the curvature of the steering region includes adjusting the curvature of the steering region by applying a tensioning force to a proximal end of the first wire such that the tensioning force is multiplied, within the steering region, by the force-multiplication arrangement.

There is further provided, in accordance with some implementations, a system, including: (1) a tube feeder, including a set of rollers, and configured to feed a tube therethrough, and/or (2) a sensor including a rider configured to roll passively in response to movement of the tube. The sensor may be configured to detect passive rolling of the rider, and/or responsively to detecting the rolling, to provide an output indicative of the movement. For some implementations, the tube feeder is sterilized.

For some implementations, at least the rider of the sensor is sterilized.

For some implementations, rolling of the rider enables verification of movement of the tube by the sensor in two degrees of motion.

For some implementations, the rider is spherical.

For some implementations, the rider is cylindrical.

For some implementations, the sensor includes an optical reader.

For some implementations, the sensor includes an electromechanical encoder.

For some implementations, the sensor is disposed downstream of the tube feeder.

There is further provided, in accordance with some implementations, apparatus, including a steering manipulator for use with a catheter including a head and a steering region, the steering manipulator including a housing, a first control unit, and/or a second control unit.

The first control unit may include: a first actuator; a first spring; a first cradle, coupled to the first actuator via the first spring, and configured to receive a first plunger of the head of the catheter; a first actuator encoder configured to provide a first actuator output indicative of a linear position of the first actuator with respect to the housing; and/or a first cradle encoder configured to provide a cradle output indicative of a linear position of the cradle with respect to the housing.

The second control unit may include: a second actuator; a second spring; a second cradle, coupled to the second actuator via the second spring, and configured to receive a second plunger of the head of the catheter; a second actuator encoder configured to provide a second actuator output indicative of a linear position of the second actuator with respect to the housing; and/or a second cradle encoder configured to provide a second cradle output indicative of a linear position of the second cradle with respect to the housing.

The steering manipulator may be configured to receive the head of the catheter in a manner that operatively couples the steering manipulator to the head, enabling the steering manipulator to manipulate the steering region by (i) actuating the first actuator to, via the first spring, slide the first plunger linearly along a stem of the head of the catheter, and/or (ii) actuating the second actuator to, via the second spring, slide the second plunger linearly along the stem.

For some implementations, linear movement of the first plunger along the stem is configured to bend the steering region.

For some implementations, linear movement of the second plunger along the stem is configured to straighten the steering region.

For some implementations, the steering manipulator further includes a third motor.

For some implementations, the third motor is configured to rotate the head.

There is further provided, in accordance with some implementations, a computer-implemented method for use with a real or simulated lung of a real or simulated subject, the method including: (1) while an imaging device, disposed at a distal portion of a catheter, is disposed within a real or simulated airway of the lung, receiving an input from the imaging device, the imaging device having a field of view; (2) referencing a three-dimensional model of the airway, and/or a planned route through the model; (3) identifying, within the model, a viewing frustum that corresponds to the field of view of the imaging device; and/or (4) generating an output that includes an output image derived from the input and/or, superimposed on the output image, an indication of a part of the planned route that appears within the viewing frustum.

For some implementations, the method is performed iteratively as the field of view changes.

For some implementations, the output is provided via a user interface to a user.

For some implementations, the input from the imaging device includes a video feed, and generating the output includes generating the output image from the video feed.

For some implementations, the part of the planned route that appears within the viewing frustum corresponds to an upcoming part of the route to be followed by a distal portion of the catheter, and/or generating the output includes superimposing on the output image the upcoming part of the planned route.

For some implementations, identifying the viewing frustum includes simulating lighting conditions corresponding to actual lighting used by the imaging device.

For some implementations, the imaging device includes a light source, and/or generating the output image includes generating the output image acquired during illumination of the airway by the light source.

For some implementations, superimposing on the output image the indication of the part of the planned route, includes superimposing a series of dots on the output image.

For some implementations, superimposing on the output image the indication of the part of the planned route, includes superimposing an arrow on the output image.

For some implementations, superimposing on the output image the indication of the part of the planned route, includes superimposing on the output image a distinct marking/coloration of the airway that lies on the part of the planned route.

For some implementations, superimposing on the output image the indication of the part of the planned route, includes superimposing on the output image a visual indication of the planned route.

For some implementations, the method further includes: referencing an expected disposition, along the route, of a representation of the imaging device; and/or responsively to the expected disposition and to the identified viewing frustum, updating the model to become more representative of the airway.

For some implementations, the method further includes determining the expected disposition responsively to receiving a sensor output from a sensor configured to sense advancement of the catheter.

For some implementations, the method further includes: referencing an expected disposition, along the route, of a representation of the imaging device; and/or responsively to the expected disposition and to the identified viewing frustum, determining a refined disposition, along the route, of the representation of the imaging device, the refined disposition being more representative, than the expected disposition, of a true position of the imaging device within the airway.

For some implementations, determining the refined disposition includes updating the model in a manner that refines the expected disposition into the refined disposition.

For some implementations, the method further includes determining the expected disposition responsively to receiving a sensor output from a sensor configured to sense advancement of the catheter.

For some implementations, identifying the viewing frustum includes: generating a set of possible viewing frustums, the set of candidate viewing frustums based on an expected disposition of the imaging device with respect to the three-dimensional model, and/or from the set of candidate viewing frustums, selecting the viewing frustum that most closely matches the field of view.

For some implementations, the expected disposition of the imaging device is based on an output from a sensor, and the method further includes identifying the expected disposition of the imaging device by reading the output of the sensor.

For some implementations, the method further includes advancing the imaging device along the planned route by manipulating a head of the catheter, the head being functionally coupled to the distal portion of the catheter via a wire.

For some implementations, manipulating the head of the catheter includes rotating the head.

For some implementations, manipulating the head of the catheter includes pulling on the wire, such that the wire changes a curvature of a distal portion of the catheter.

For some implementations: the method further includes generating, from the viewing frustum, a derived image, and/or identifying the viewing frustum includes matching the derived image to the input from the imaging device.

For some implementations: identifying the viewing frustum includes selecting the viewing frustum from a plurality of candidate viewing frustums within an expected range of the three-dimensional model, each of the candidate viewing frustums having a corresponding derived image, and/or selecting the viewing frustum from the plurality of candidate viewing frustums includes comparing, with the input from the imaging device, the derived image of each of the candidate viewing frustums.

For some implementations, generating the derived image includes calculating light reflections from a virtual light source within the model in a manner that the derived image matches the input from the imaging device.

For some implementations: the imaging input is a three-dimensional image acquired in real time, and/or comparing image inputs acquired iteratively by the imaging device includes comparing image inputs in a manner that creates a real-time three-dimensional representation of the airway.

There is further provided, in accordance with some implementations, a system, including a catheter, a manipulator assembly, and/or a control system. The catheter may include a head, at a proximal portion of the catheter, and/or a tube, extending from the head toward a distal portion of the catheter. The manipulator assembly may be configured to receive and engage the catheter in a manner that configures the manipulator assembly to manipulate the distal portion of the catheter through a real or simulated airway of a real or simulated subject. The control system may be configured to electronically operate the manipulator assembly, and may include a data-processing system including means for carrying out the steps in paragraph 374.

For some implementations, the catheter is sterilized.

For some implementations, the system further includes an imaging device, positionable at the distal portion of the catheter.

For some implementations, the system further includes a sensor, configured to sense manipulation of the catheter; and/or to provide: an advancement output indicative of a sensed linear manipulation of the catheter; and/or a rotation output indicative of a sensed rotational manipulation of the catheter.

For some implementations, the catheter is a first catheter; the manipulator assembly is a first manipulator assembly; the system further includes a second catheter and a second manipulator assembly; the first manipulator assembly is configured to receive and engage the first catheter in a manner that configures the first manipulator assembly to manipulate a distal portion of the first catheter through the airway; and/or the second manipulator assembly is configured to receive and engage the second catheter in a manner that configures the second manipulator assembly to manipulate a distal portion of the second catheter through the airway.

For some implementations, the system further includes a first sensor configured to sense the manipulation of the first catheter by the first manipulator assembly, and/or a second sensor configured to sense the manipulation of the second catheter by the second manipulator assembly,
and, for each of the first sensor and the second sensor, a sensor output reflective of the manipulation of the respective catheter is provided to the control system, and the control system is configured to use the sensor output to update the generated output.

There is further provided, in accordance with some implementations, a computer-implemented method for use with an imaging device within a real or simulated airway of a real or simulated lung of a real or simulated subject, the method including: (1) while the imaging device is disposed at a first site within the airway such that the imaging device has a first field of view, receiving a first input from the imaging device; (2) identifying, within a three-dimensional model of the airway, a first viewing frustum that corresponds to the first field of view; (3) while the imaging device is disposed at a second site having a second field of view, receiving a second input from the imaging device; (4) identifying, within the three-dimensional model, a second viewing frustum that corresponds to the second field of view; and/or (5) responsively to identifying the first viewing frustum and the second viewing frustum, adjusting at least a part of the three-dimensional model.

For some implementations, adjusting at least a part of the three-dimensional model includes adjusting the part of the three-dimensional model between the first viewing frustum and the second viewing frustum.

For some implementations, adjusting the part of the three-dimensional model includes scaling the three-dimensional model to match a difference in scale between the part of the model, and at least one of the first field of view and the second field of view.

For some implementations, adjusting the part of the three-dimensional model includes rotating a depiction of the airway within the model.

For some implementations, the method is performed iteratively, the computer receiving iterative inputs from the imaging device as the imaging device moves along the airway.

For some implementations, a distance between the first field of view and the second field of view includes a part of a planned route through the model, and adjusting the part of the model includes adjusting a part of the planned route through the model.

For some implementations, adjusting the part of the planned route includes adjusting a distance between subsequent forks of the airway.

For some implementations, adjusting the part of the planned route includes adjusting an angle between subsequent forks of the airway.

For some implementations, the method further includes referencing a planned route through the model; and/or subsequently to adjusting at least the part of the three-dimensional model, and while the imaging device remains at the second site, generating an output that includes (i) an output image derived from the second input, and/or (ii) an indication of a part of the planned route that appears within the second viewing frustum.

For some implementations, the method further includes providing a display of at least part of the planned route through a corresponding part of the three-dimensional model.

There is further provided, in accordance with some implementations, a computer-implemented method for use with a real or simulated lung of a real or simulated subject, the method including: (1) while an imaging device, disposed at a distal portion of a catheter, is disposed in a true disposition within a real or simulated airway of the lung, receiving an input from the imaging device, the imaging device having a field of view; (2) referencing a three-dimensional model of the airway, a planned route through the model, and/or an expected disposition, along the route, of a representation of the imaging device; (3) identifying, within the model, a viewing frustum that corresponds to the field of view of the imaging device, and/or (4) responsively to the identified viewing frustum, determining a refined disposition, along the route, of the representation of the imaging device, the refined disposition being more representative, than the expected disposition, of the true disposition.

For some implementations, the true disposition includes a true position and a true orientation, and being disposed in the true disposition includes being disposed in the true position and true orientation.

For some implementations, the refined disposition includes a refined position and a refined orientation, and determining the refined disposition includes determining the refined position and refined orientation of the representation of the imaging device.

For some implementations, the expected disposition includes an expected disposition and an expected orientation, and referencing the expected disposition includes referencing the expected disposition and expected orientation of the representation of the imaging device.

For some implementations, the method includes referencing a portion of the three-dimensional model within a threshold distance of the expected disposition, and within the threshold distance, matching the viewing frustum to the field of view.

For some implementations, identifying the viewing frustum includes identifying similar anatomical features in the received input and in a referenced portion of the three-dimensional model.

For some implementations, determining the refined disposition of the imaging device provides a refined indication of a disposition of the distal portion of the catheter.

For some implementations, the method further includes generating an output that includes: an input image derived from the input from the imaging device, and/or superimposed on the input image, an indication of a part of the planned route that appears within the viewing frustum.

For some implementations, the planned route is determined by an algorithm preoperatively; and/or the method further includes guiding a distal steering region of the catheter according to the planned route preoperatively determined by the algorithm.

For some implementations, the planned route is determined by an operator intraoperatively; and/or the method further includes guiding a distal steering region according to the planned route intraoperatively determined by the operator.

For some implementations, the method further includes: receiving a position input from a sensor indicative of a linear and rotational position of the imaging device with respect to the airway; and/or and determining the refined disposition of the representation of the imaging device includes refining the linear and rotational disposition of the representation the imaging device with respect to the model.

For some implementations, receiving the position input from the sensor includes receiving input from the sensor indicative of a linear and rotational disposition of the imaging device.

For some implementations, receiving the position input from the sensor includes, using the sensor, measuring a distance traveled by, and rotation of, the imaging device.

For some implementations, the method further includes calculating a quantitative difference between the refined disposition and the expected disposition.

For some implementations, the quantitative difference is provided in three dimensions, such that calculating the quantitative difference includes calculating the quantitative difference in three dimensions.

For some implementations, the method further includes outputting the quantitative difference as an adjustment to the three-dimensional model.

For some implementations, the method is performed iteratively as the catheter follows the planned route to a target, such that receiving the input from the imaging device includes receiving a video feed.

For some implementations, in at least some iterations of the method, the expected disposition differs from the true disposition, such that determining the refined disposition requires an adjustment to the model.

For some implementations, the required adjustments improve accuracy of the model, such that the refined disposition approaches the true disposition as the catheter approaches the target.

For some implementations, the method further includes determining the expected disposition of the representation of the imaging device based on receiving input from a sensor.

For some implementations, referencing the expected disposition includes referencing the expected disposition derived by a controller with respect to sensor input indicative of a distance traveled along the planned route.

For some implementations, the method further includes displaying an indication of the refined disposition by the controller.

There is further provided, in accordance with some implementations, a system, including a catheter, an imaging device, a manipulator structure, and/or a control system. The catheter may include a head, at a proximal portion of the catheter; and/or a tube, extending from the head toward a distal portion of the catheter. The manipulator structure may include (i) a manipulator assembly configured to receive and engage the catheter in a manner that configures the manipulator assembly to manipulate the distal portion of the catheter through a real or simulated airway of a real or simulated subject; and/or a sensor, configured to sense the manipulation of the catheter by the manipulator assembly, and/or provide a manipulation output indicative of the sensed manipulation of the catheter. The control system may be configured to electronically operate the manipulator structure; receive the manipulator output; and/or may include a data-processing system including means for carrying out the steps in paragraph 416, the control system determining the expected disposition responsively to the manipulation output.

For some implementations, the catheter is sterilized.

For some implementations, the sensor is configured to measure movement of the catheter by determining a length of the tube moving past a reference point on the manipulator assembly; and/or rotation of the tube from an initial position.

For some implementations, the manipulation output includes a measurement of a distance traveled by a distal end of the tube.

For some implementations, the manipulation output includes a measurement of rotation of the tube as determined by determining a degree of rotation of the head.

For some implementations, the control system is configured to verify an expected disposition of the distal portion of the catheter in the airway.

For some implementations, the imaging device used for a distal part of the operative plan is an ultrasound probe.

For some implementations, the ultrasound probe is configured to produce planar images.

For some implementations, determination of the expected disposition is based on three-dimensional reconstruction of a plurality of stacked planar images acquired intraoperatively.

For some implementations, the system is configured to align/stack the planar images to produce a three-dimensional image intraoperatively.

For some implementations, the system further includes an imaging device, positionable at the distal portion of the catheter.

For some implementations, the imaging device is an ultrasound transceiver.

For some implementations, the imaging device is a camera.

For some implementations, the camera has a fiber optic light source.

For some implementations, the camera is configured to provide a video output of a position of a distal end of the catheter.

For some implementations, the catheter is a first catheter; the system further includes a second catheter; the manipulator assembly is a first manipulator assembly; the system further includes a second manipulator assembly; the sensor is a first sensor; the manipulation output is a first manipulation output; the system further includes a second sensor; the second manipulator assembly is configured to receive and engage the second catheter in a manner that configures the second manipulator assembly to manipulate a distal portion of the second catheter through the airway; and/or the second sensor is configured to (i) sense the manipulation of the second catheter by the second manipulator assembly; and/or (ii) provide a second manipulation output indicative of the sensed manipulation of the second catheter.

For some implementations, the first catheter is configured to carry a tool; the second catheter is configured to carry an imaging device; the first manipulation output provides information on the expected disposition of the tool; and/or the second manipulation output provides information on the expected disposition of the imaging device.

For some implementations, the control system is configured to record a tool site for the tool; compare the first manipulation output with the tool site; and/or provide a tool comparison output indicating the expected disposition of the tool relative to the tool site.

For some implementations, the control system is configured to record an imaging site for the imaging device; compare the second manipulation output with the imaging site; and/or provide an imaging comparison output indicating the expected disposition of the imaging device relative to the imaging site.

There is further provided, in accordance with some implementations, a computer-implemented method for use with a real or simulated lung of a real or simulated subject, the method including: (1) while an imaging device, disposed at a distal portion of a catheter, is disposed in a true position within a real or simulated airway of the lung, receiving an input from the imaging device; (2) referencing a three-dimensional model of the airway, a planned route through the model, and/or an expected disposition, along the planned route, of a representation of the imaging device; and/or (3) determining a refined position, along the route, of the representation of the imaging device, the refined position being more representative, than the expected disposition, of the true position.

There is further provided, in accordance with some implementations, a system for performing a bronchoscopic procedure on a real or simulated lung of a real or simulated subject, the system including a catheter, an extracorporeal manipulator structure, and/or a control system. The catheter may include a head and/or a tube that has a steering region at a distal portion thereof. The extracorporeal manipulator structure may include a manipulator assembly and a sensor; and/or may be configured to receive the catheter such that the manipulator assembly becomes engaged with the catheter. The sensor may be configured to sense linear advancement of the catheter, and/or provide an advancement output indicative of the sensed linear advancement. The control system may be electronically connectable with the manipulator structure, and may include a data-processing system that includes a manipulator module, a tracing module, and/or a display module.

The manipulator module may be configured to instruct the manipulator assembly to linearly advance the catheter.

The tracing module may be configured to reference a three-dimensional model of a real or simulated lung of a real or simulated subject, the model including a representation of a real or simulated airway of the lung, receive the advancement output, and/or responsively to the advancement output, determine an expected disposition within the representation of the airway corresponding to a disposition of the distal portion of the catheter along the airway.

The display module may be configured to provide a navigational guide responsively to the disposition determined by the tracing module.

For some implementations, the system further includes (i) an imaging device having a field of view, and being extendable through the catheter, and/or a model-image bridging module.

The model-image bridging module may be configured to, while the distal portion of the tube is disposed within the airway and the imaging device is disposed at the distal portion of the tube: (i) reference a planned route through the model, (ii) receive an imaging input from the imaging device, (iii) identify, within the model, a viewing frustum that corresponds to the field of view of the imaging device, and/or (iv) responsively to the expected disposition and the identified viewing frustum, refine the expected disposition into a refined disposition. The navigational guide may include an output that includes an output image derived from the imaging input, and/or superimposed on the output image, an indication of a part of the planned route that appears within the viewing frustum, positioning of the indication with respect to the output image being responsive to the refined disposition.

For some implementations, the catheter is sterilized.

For some implementations, the advancement output includes a measurement of a distance traveled by a distal end of the tube as determined by a length of the tube moving past a reference point on the manipulator assembly.

For some implementations, the advancement output is determined by mechanical contact of the sensor with the tube.

For some implementations, the sensor is an electromechanical sensor.

For some implementations, sensing of linear advancement is via optical observation of the tube.

For some implementations, the sensor is configured to record and output a length of tube passing the sensor.

For some implementations, the navigational guide includes an indication of the planned route beyond the identified viewing frustum.

For some implementations, the navigational guide includes an indication of a part of a planned route distal to the refined disposition.

For some implementations, the sensor is positioned in proximity to an entry point of the tube to the subject.

For some implementations, the sensor is configured to track and output rotation of the tube.

For some implementations, the control system is configured to use the advancement output to adjust a position of the steering region.

For some implementations, the sensor is configured to generate output indicative of a position of the steering region in three-dimensional space.

For some implementations, a tool is disposed at a distal end of the catheter, and the system is configured to verify a position of the tool.

For some implementations, the manipulator structure further includes a rotation sensor configured to sense rotation of the catheter, and/or provide a rotation output indicative of the sensed rotation. The tracing module may be configured to receive the rotation output, and/or determine the expected disposition responsively to the advancement output and the rotation output.

For some implementations, the navigational guide includes a rotation indication that is indicative of the rotation output.

For some implementations, the manipulator structure further includes a bending sensor configured to sense bending of the catheter, and/or provide a bending output indicative of the sensed bending. The tracing module may be configured to receive the bending output, and/or determine the expected disposition responsively to the advancement output and the bending output.

For some implementations, the navigational guide includes a bending indication that is indicative of the bending output.

For some implementations, the manipulator assembly includes a steering manipulator and an advancement manipulator, and the manipulator assembly becomes engaged with the catheter by the steering manipulator receiving the head and the advancement manipulator receiving the tube of the catheter.

For some implementations, the sensor is further configured to sense rotation of the tube and provide a rotational output indicative of the sensed rotation.

For some implementations, the steering manipulator is configured to sense bending of the tube and to provide a bending output indicative of the sensed bending.

For some implementations, the tracing module is configured to receive the advancement output, the rotational output, and the bending output, and/or determine the disposition responsively to the advancement output, the rotational output, and the bending output.

For some implementations, the manipulator structure further includes a force sensor configured to sense force exerted on the steering region, and to provide a force output indicative thereof.

For some implementations, the control system further includes means to maintain a bending state of the steering region by automatically responding to the force output.

For some implementations, the system further includes: an imaging device, positionable at the distal portion of the catheter; and/or a data-processing system including means for carrying out the steps in paragraph 374.

For some implementations, the control system includes the data-processing system, configured to electronically operate the extracorporeal manipulator structure.

For some implementations, the system further includes: an imaging device, positionable at the distal portion of the catheter; and/or a data-processing system including means for carrying out the steps in paragraph 416.

For some implementations, the control system includes the data-processing system, configured to electronically operate the manipulator structure.

For some implementations, the catheter further includes a first wire, and a second wire, each of the first wire and the second wire extending from the steering region proximally along the tube, and the head further includes: a stem; a first plunger, operatively coupled to the steering region by being attached to the first wire, and mounted on the stem to be slidable linearly along the stem; and/or a second plunger, operatively coupled to the steering region by being attached to the second wire, and mounted on the stem to be slidable linearly along the stem independently of the first plunger.

For some implementations, the steering manipulator is configured to bend the steering region by sliding the first plunger.

For some implementations, the steering manipulator is configured to straighten the steering region by sliding the second plunger.

There is further provided, in accordance with some implementations, a system including a catheter and/or a robot. The catheter may include a head at a proximal region of the catheter, and/or a tube. The tube may have a distal portion configured to be advanced into the subject via a body orifice of the subject, and/or a steering region at the distal portion. The robot may include a manipulator assembly.

The manipulator assembly may include a steering manipulator and/or an advancement manipulator. The manipulator assembly may be configured to be loaded with the catheter such that (i) the steering manipulator receives the head in a manner that operatively couples the steering manipulator to the steering region such that a curvature of the steering region is adjustable by the steering manipulator manipulating the head, and/or the advancement manipulator receives the tube such that operation of the advancement manipulator feeds the tube through the advancement manipulator in a manner that (i) pulls the head and the steering manipulator distally toward the advancement manipulator and the body orifice, and (ii) pushes the tube distally through the body orifice into the subject.

There is further provided, in accordance with some implementations, a method for use with a real or simulated subject, the subject having a real or simulated body orifice. The method may include, into an advancement manipulator of a manipulator assembly, loading a tube of a catheter, the tube having a steering region at a distal portion of the tube. The method may include loading a head of the catheter into a steering manipulator of the manipulator assembly, the head being at a proximal region of the catheter, and the steering manipulator being configured to adjust a curvature of the steering region by manipulating the head. The method may include operating the advancement manipulator to feed the tube through the advancement manipulator in a manner that (i) pulls the head and the steering manipulator distally toward the advancement manipulator and the body orifice, and/or (ii) pushes the tube distally through the body orifice into the subject.

There is further provided, in accordance with some implementations, a method for use with a real or simulated subject, the subject having a real or simulated body orifice, the method including (a) positioning an advancement manipulator of a manipulator assembly at the body orifice; and/or (b) while (i) a tube of a catheter is disposed through the advancement manipulator, and (ii) a head of the catheter is disposed within a steering manipulator of the advancement assembly, operating the advancement manipulator to feed the tube through the advancement manipulator in a manner that (i) pulls the head and the steering manipulator distally toward the advancement manipulator and the body orifice, and (ii) pushes the tube distally through the body orifice into the subject.

The present invention will be more fully understood from the following detailed description of implementations thereof, taken together with the drawings, in which:

### BRIEF DESCRIPTION OF THE DRAWINGS

FIGS. 1, 2A-C, 3, 4, 5A-C, 6A-B, 7A-D, 8A-C, and 9A-H are schematic illustrations of a robotic system, and components thereof, for advancing and/or steering a catheter, in accordance with some implementations of the present disclosure;

FIGS. 10A-B are schematic illustrations of a sensor for tracking advancement and/or rotation of a catheter, in accordance with some implementations of the present disclosure;

FIGS. 11A-C and 12 are schematic illustrations of variants of manipulator structures of robotic systems, in accordance with some implementations of the present disclosure;

FIGS. 13A-B are diagrams showing exemplary techniques for planning routes for catheters through airways, in accordance with some implementations;

FIG. 14 is a schematic illustration of an exemplary system for guiding a catheter in an endoscopic procedure;

FIG. 15 is a schematic inputs and outputs of an exemplary robotically-controlled catheter-guidance system;

FIGS. 16A-C are diagrammatic illustrations of a representative progression and visualization of steps in a robotically-controlled endoscopic procedure;

FIG. 17 is a schematic display of inputs and outputs of an exemplary robotically-controlled catheter-guidance system;

FIGS. 18A-E and 19A-B are diagrammatic illustrations of a representative progression and visualization of steps in a robotically-controlled endoscopic procedure to refine an expected disposition of a catheter tip;

FIG. 20 is a schematic illustration of an exemplary system for guiding two catheters in a robotically-controlled endoscopic procedure;

FIGS. 21, 22A-B, and 23A-C are schematic illustrations of a steering manipulator and a catheter for use therewith, in accordance with some implementations; and

FIGS. 24A-F, 25, 26A-D, 27A-B, 28A-B, and 29A-B are schematic illustrations of, inter alia, an advancement unit and rollers, in accordance with some implementations.

### DETAILED DESCRIPTION

The present disclosure includes different variants of some elements. Variants of a given element typically have the same structure and/or function as each other except for any differences described. For any given element for which different variants are disclosed, the identical name is used for each variant, in order to denote that they are, in fact, variants the same given element. Unless stated otherwise, implementations of the devices, systems, and techniques described herein may include any arrangement in which one variant of an element is substituted with another identically-named variant of that element. Furthermore, throughout the figures, suffixes are used to denote different variants of the same element. Unless stated otherwise, such variants may be substituted with each other, mutatis mutandis. That is, unless stated otherwise, any element having a given reference numeral may be substituted with any other element (i.e. any other variant of the element) having the same reference numeral, independent of any suffix. For example, an element having a reference numeral suffixed with a double prime symbol (″) is a variant of, and may be substituted with, another element having the same reference numeral suffixed with a single prime symbol (′) or with no suffix.

In order to avoid undue clutter from having too many reference numbers and lead lines on a particular drawing, some elements are introduced via one or more drawings and not explicitly identified in every other drawing that contains that element.

The described systems, apparatuses, devices, methods, etc. should not be construed as limiting in any way. Instead, the present disclosure is directed toward all novel and nonobvious features and aspects of the various disclosed implementations and implementations, alone and in various combinations and sub-combinations with one another. The disclosed systems, apparatuses, devices, methods, etc. are not limited to any specific aspect, feature, or combination thereof, nor do the disclosed systems, apparatuses, devices, methods, etc. require that any one or more specific advantages be present or problems be solved.

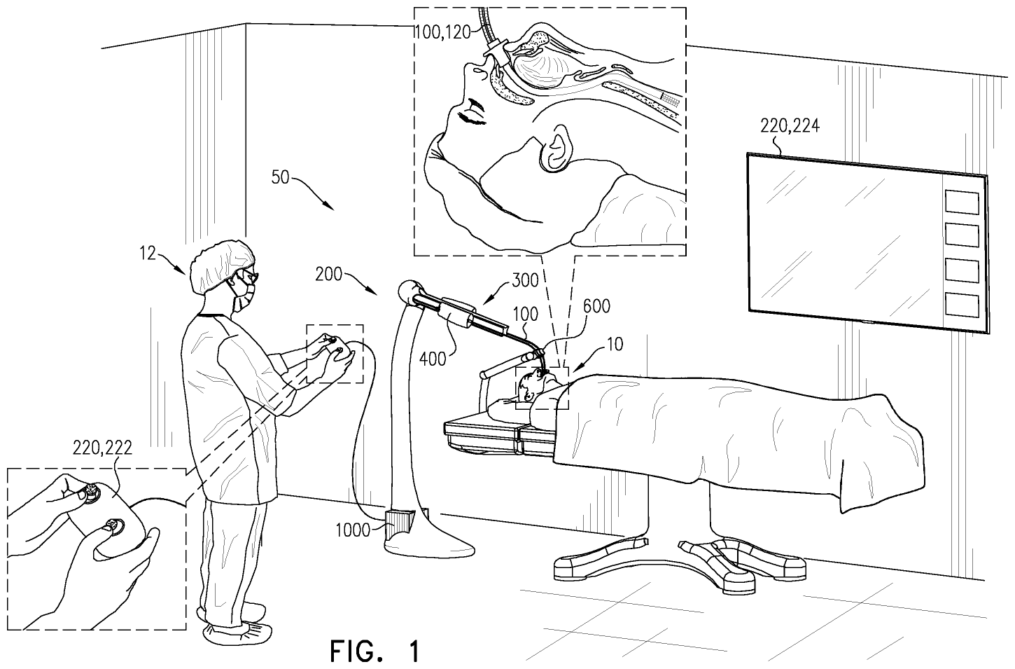

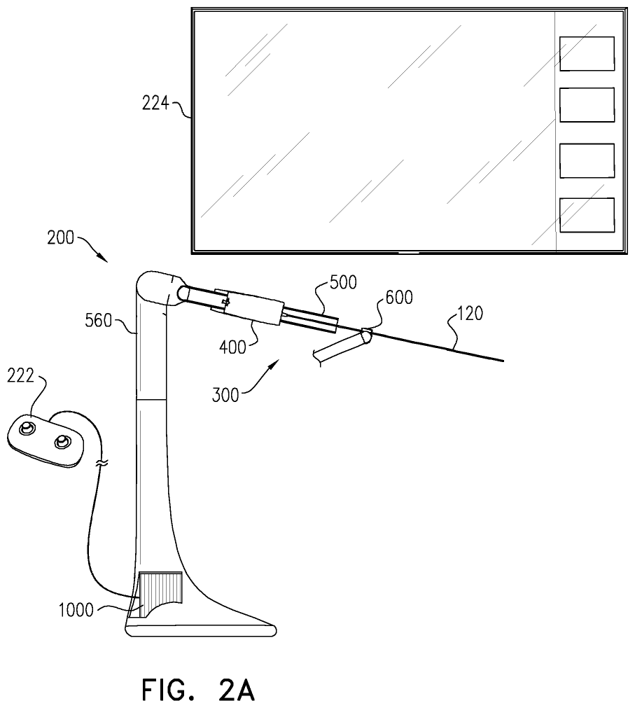

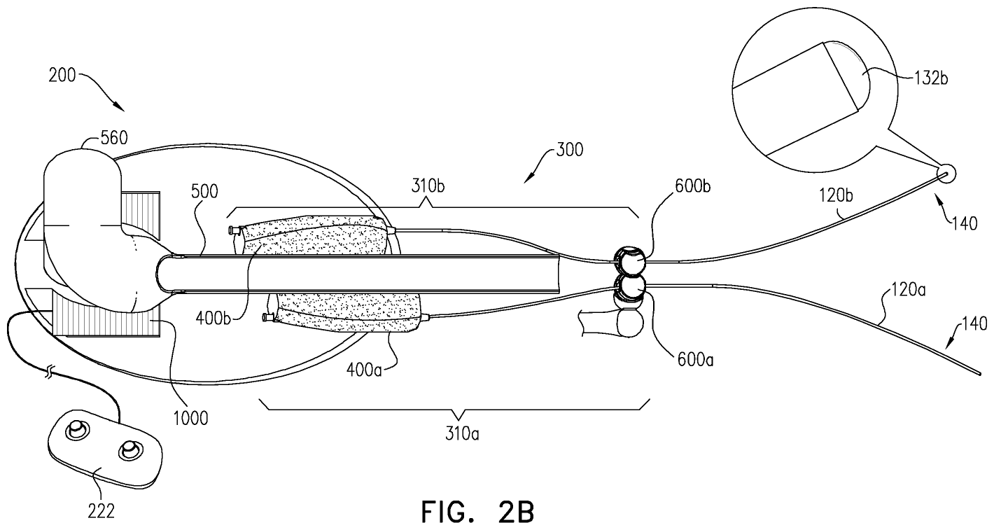

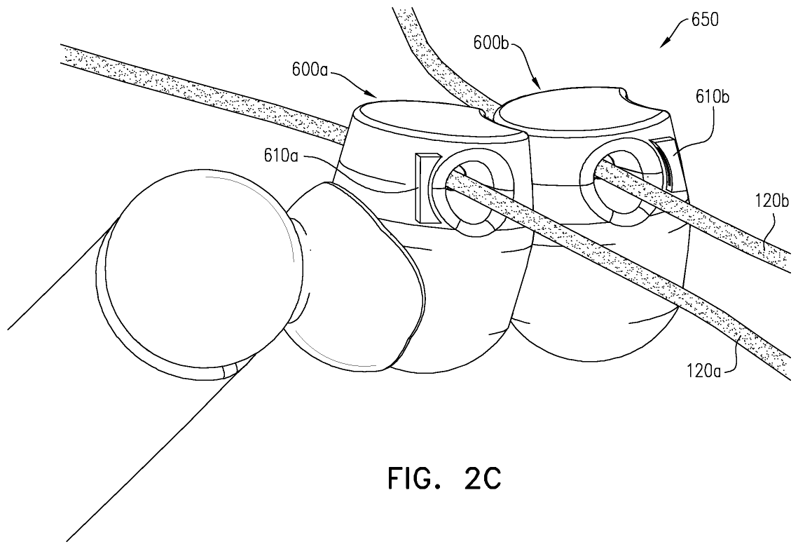

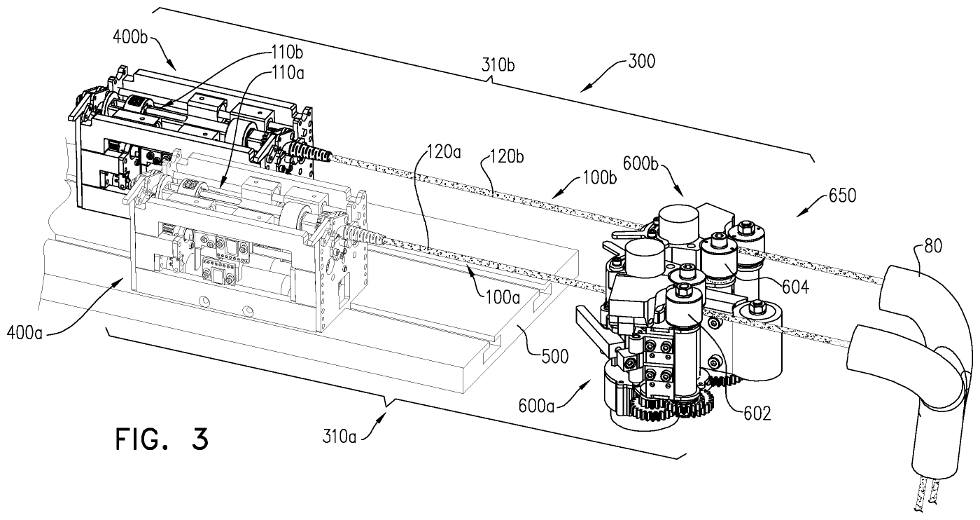

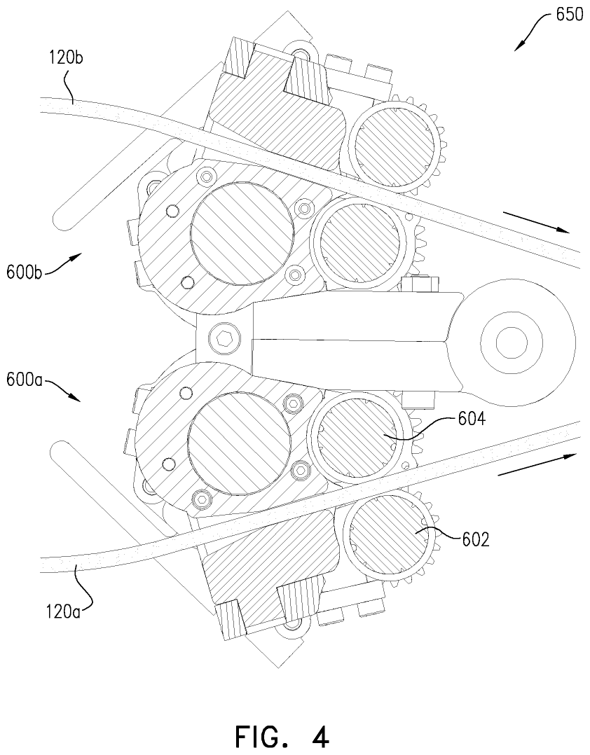

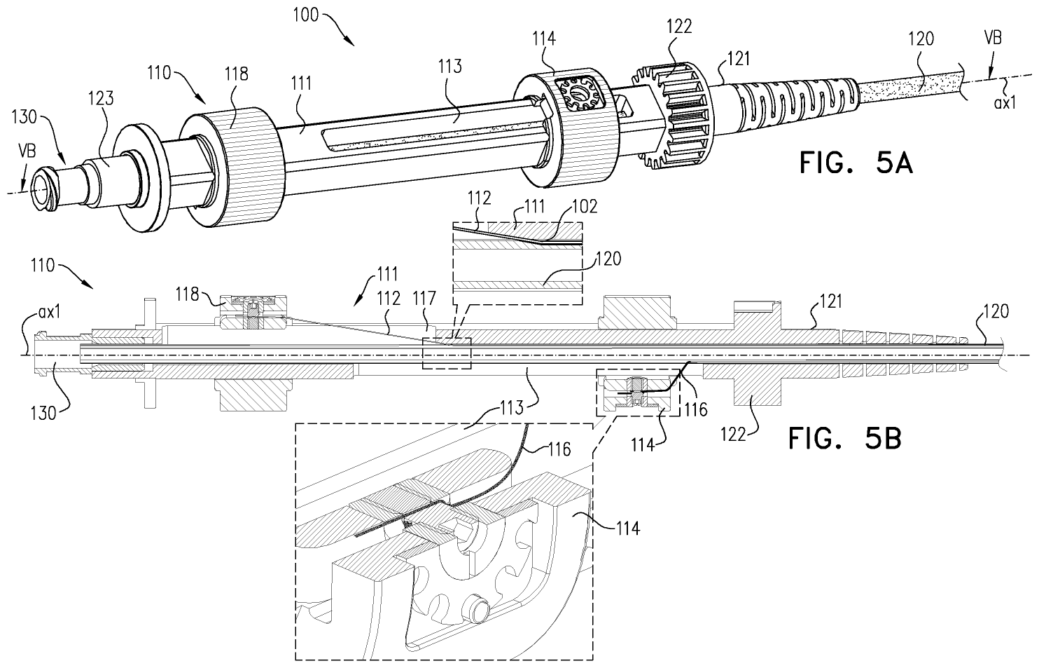

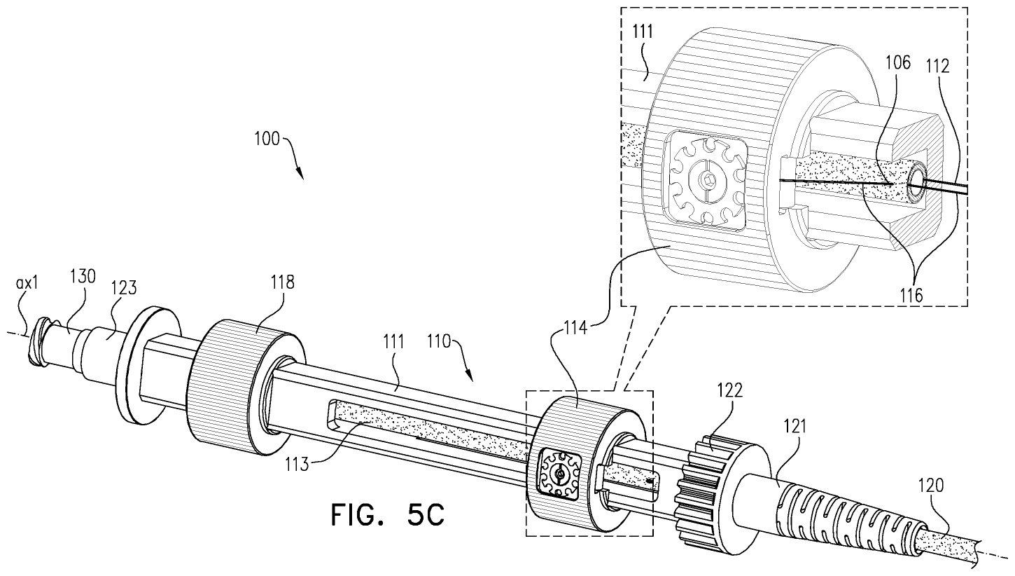

Reference is made to FIGS. 1, 2A-C, 3, 4, 5A-C, 6A-B, 7A-D, 8A-C, and 9A-H, which are schematic illustrations of an exemplary system 50, in accordance with some implementations. System 50 comprises one or more catheters 100, and a robot 200 for controlling advancement and manipulation of the catheters into and within a subject 10. Typically, system 50 is for performing a bronchoscopic procedure on a lung 20 of a subject, as shown in FIG. 1. However, system 50, or variants thereof, may be used for performing other procedures at other anatomical sites and/or via other routes.

Robot 200 comprises a manipulator structure 300 that comprises one or more manipulator assemblies 310 (e.g. a manipulator assembly 310a and/or a manipulator assembly 310b)—e.g. see FIGS. 2B and 3. In some implementations, and as shown, manipulator structure 300 may comprise two manipulator assemblies 310. In some implementations, the manipulator structure may comprise three or more manipulator assemblies. Each manipulator assembly 310 may comprise a steering manipulator 400 and an advancement manipulator 600, which are configured to cooperatively control a respective catheter 100, e.g. as further described herewithin below. Steering manipulator 400 is schematically illustrated in, inter alia, FIGS. 7A-D. Advancement manipulator 600 is schematically illustrated in, inter alia, FIG. 4.

Catheter 100 comprises a head 110 at a proximal part of the catheter, and a tube 120 extending distally away from the head—e.g. see FIGS. 5A-C and 6A-B. Head 110 is configured to fit within steering manipulator 400 (e.g. as shown in FIGS. 8A-C), enabling the steering manipulator to control a steering region 140 of catheter 100 (e.g. as shown in FIGS. 9A-H).

Robot 200 further comprises a control system 1000 that, inter alia, controls manipulator structure 300 (e.g. manipulator assemblies 310 thereof). Control system 1000 may comprise one or more data-processing systems (DPSs). In some implementations, and as shown (e.g. in FIGS. 1 and 2A), robot 200 may be provided with a user interface 220, e.g. comprising a controller 222 (e.g. a hand-operated controller), which an operator 12 may use to operate manipulator structure 300 (e.g. via control system 1000) to control steering region 140. Controller 222 may be considered to be a Human Interface Device (HID). In the example shown, controller 222 is illustrated as a hand-held controller, similar to a video console's gamepad. However, it is to be understood that various configurations are possible. In addition to or instead of hand-control 220, system 50 may comprise a wireless user interface and a remote computing unit (not shown). Visual representation of steering region 140 and data related to the manipulation thereof may be viewed, e.g. on a screen 224, which may be a component of user interface 220.

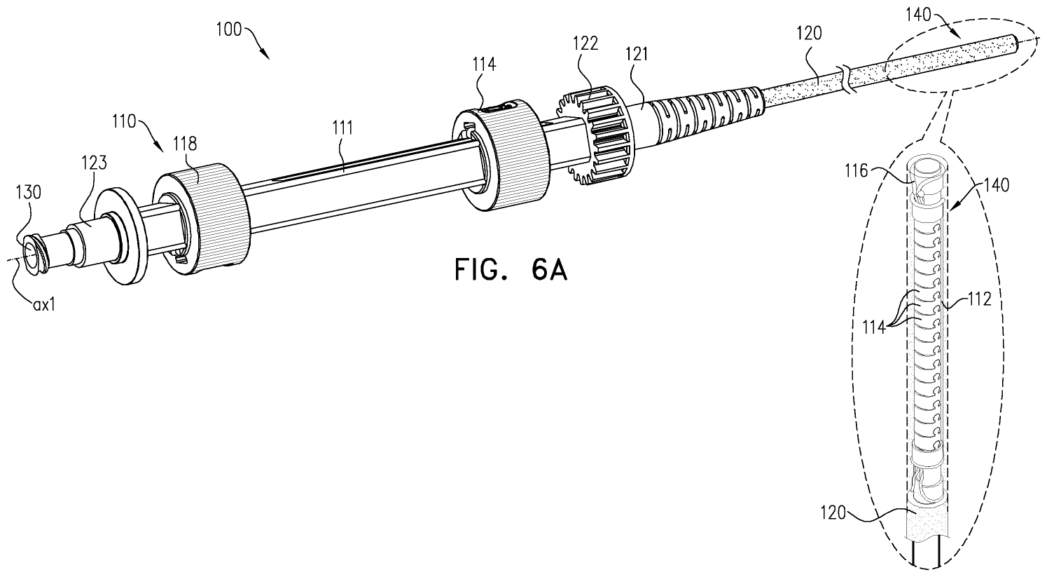

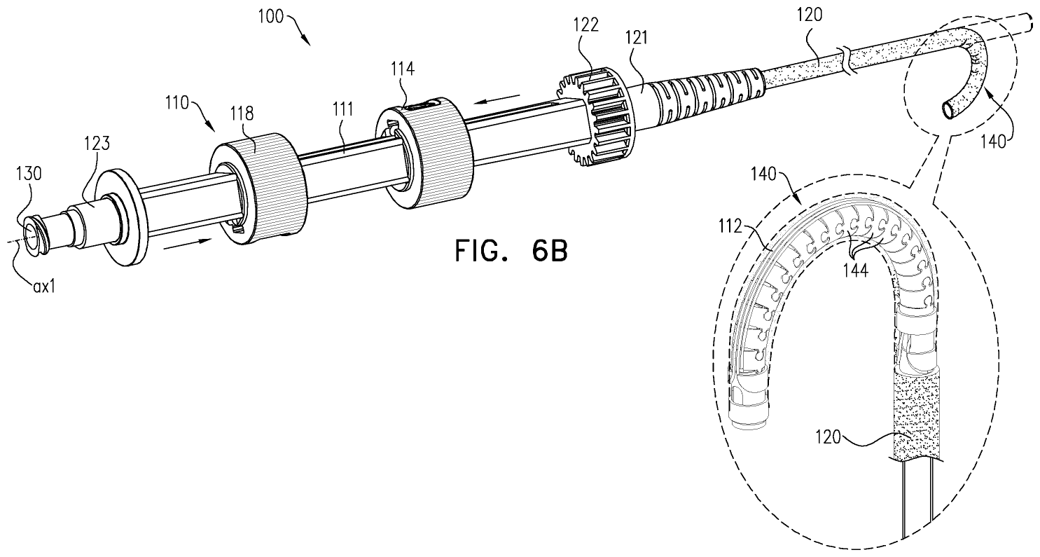

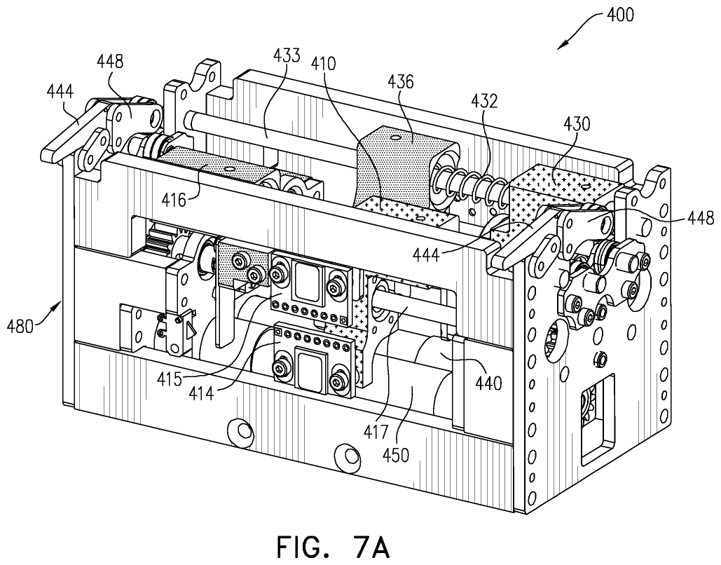

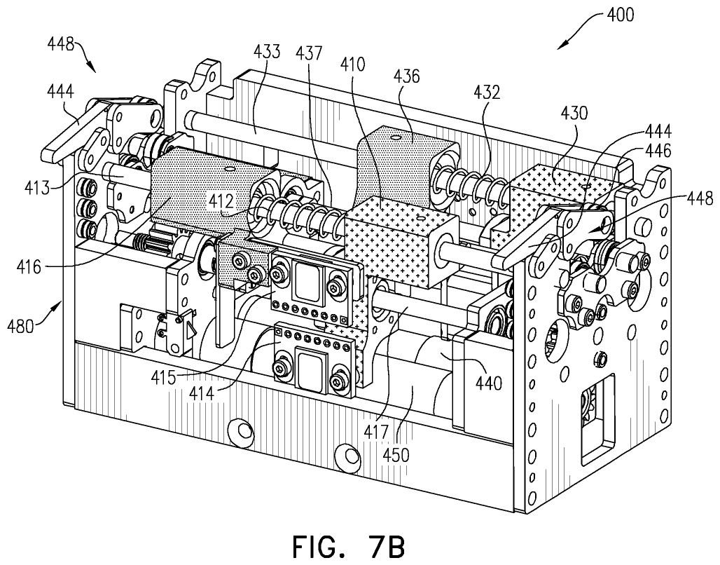

In some implementations, and as shown, robot 200 (e.g. manipulator structure 300 thereof, e.g. manipulator assembly 310 thereof, such as advancement manipulator 600 thereof) may comprise a sensor configured to sense (e.g. track) either or both advancement and rotational movement of tube 120. FIG. 2C shows an example of such a sensor, embodied as sensor a 610, and FIGS. 10A-B show another example of such a sensor, embodied as sensor 620.

Manipulator structure 300 typically defines an advancement path 500. For example, manipulator structure 300 may comprise a track (e.g. a rail) that defines advancement path 500, as shown, e.g. in FIG. 2B. Advancement path 500 may be coupled to a mount 560, configured to support robot 200.

Throughout this implementation, the suffixes a and b after a number refer to duplicate components of the same design, which are configured to act in tandem as part of a system, to manipulate a pair of catheters. In any given illustrated process or stage of operation, each equivalent component a, b may be shown in a different state, as each catheter is configured to be manipulated independently.

Typically, and as shown in, e.g. FIG. 2B, manipulator structure 300 comprises two manipulator assemblies 310 (e.g. a first manipulator assembly 310a, and a second manipulator assembly 310b), for use with a corresponding two catheters 100 (e.g. a first catheter 100a and a second catheter 100b). That is, a first head 110a may be fitted within steering manipulator 400a, and a second head 110b fitted within steering manipulator 400b. Thus, system 50 may comprise two catheters 100. This arrangement may be advantageous, e.g. in implementations in which system 50 is configured to perform a bronchoscopic procedure using a dual access approach.

For some implementations, advancement manipulator 600 (e.g. each advancement manipulator) is configured as a single unit to advance a single catheter tube. Alternatively, and as shown, robot 200 (e.g. manipulator structure 300) may comprise an advancement unit 650 (e.g. a double unit) that comprises multiple (e.g. two) advancement manipulators 600 (e.g. advancement manipulator 600a and advancement manipulator 600b) coupled to each other—e.g. fixedly attached or adjustably coupled to each other. Advancement unit 650 may be fitted with one or more sensors 610 (e.g. a sensor 610a and/or a sensor 610b) to sense advancement and/or rotation of tube 120a, 120b. Sensor 610 may be an optical sensor that optically detects advancement and/or rotation of tube 120.

In some implementations, system 50 may comprise a sheath 80, through which tubes 120 may be passed into the trachea. In some implementations, and as shown in FIG. 3, sheath 80 is forked to facilitate convergence of two tubes 120 for advancement into the trachea.

In some implementations, the catheter may be provided as part of system 50. In some implementations, robot 200 may be provided without catheters 100. In some implementations, catheters 100 may be provided without the robot—e.g. as single-use consumables, such as in a sterilized (or sterilizable) packaging. In some such implementations, the catheter may be configured to lock into the proximal steering manipulator 400, as tube 120 passes through the distal advancement manipulators 600, respectively, as further described below.

In some implementations, and as shown (FIG. 1), steering manipulators 400 and advancement path 500, e.g. a track, are positionable at least partly independently of the advancement manipulators 600. For example, the steering manipulators and/or the track may be floor-mounted or ceiling-mounted, with advancement manipulators being mounted in a different manner. For example, the advancement manipulator may be table-mounted. Alternatively, the steering manipulators 400, the track, and the advancement manipulator may all be secured to mount 560—e.g. with one or more of these component being secured to the mount via adjustable arms. FIGS. 2A-C show an example in which advancement manipulators 600 are mounted on an adjustable arm.

Advancement manipulators 600 may be positioned close to the body orifice into which catheters 100 are to be advanced—e.g. typically the mouth in the case of bronchoscopic procedures. For example, the advancement manipulators may be positionable less than 10 cm (e.g. 1-10 cm), such as less than 5 cm (e.g. 1-5 cm), such as less than 3 cm (e.g. 1-3 cm) from the body orifice. For some implementations, the advancement manipulators are placeable in contact with the subject proximate the body orifice. For some implementations, the advancement manipulators can be rested against and/or secured to the subject in such a position.

As described in more detail hereinbelow, each advancement manipulator 600 is configured to advance the tube 120 of a corresponding catheter 100 into the subject by feeding the tube through the advancement manipulator. Positioning advancement manipulator 600 close to the body orifice reduces the length of tube 120 that is disposed between the advancement manipulator and the body orifice, thereby advantageously preventing buckling of the tube that might otherwise occur due to pushing by the advancement manipulator, and therefore advantageously increasing the degree to which the distance by which the advancement manipulator advances the tube corresponds to the distance that the distal end of the tube has advanced into the subject (e.g. along an airway). This may be further facilitated by the overall structure of the manipulator structure, by which the steering manipulator responsively moves along the advancement path to accommodate (e.g. is drawn along the advancement path by) the advancement of the catheter—e.g. as described in more detail hereinbelow.

For some implementations, the ability to position the advancement manipulators close to the body orifice is facilitated by the ability to position the advancement manipulators at least partly independently of the steering manipulators.

As shown in FIGS. 5A-C, catheter 100 may comprise a head 110 and a flexible tube 120. Tube 120 has a steering region 140—e.g. at a distal portion thereof (not shown in FIGS. 5A-C). Head 110 is operatively coupled to steering region 140, e.g. via one or more wires 112, 116 (e.g. pullwires), such that bending of the steering region is controllable by manipulating the head.

For some implementations, head 110 comprises a stem 111, and one or more plungers slidably coupled to (e.g. mounted on) the stem. In the example shown, head 110 comprises two plungers 114, 118, and catheter 100 (e.g. tube 120) comprises two corresponding wires. Head 110 may be elongate, defining a head axis ax1—e.g. on which stem 111 lies. Plunger 114 is connected to a wire 116, and plunger 118 is connected to a wire 112. Wire 116 operatively couples plunger 114 to steering region 140 of tube 120, such that sliding of plunger 114 pulls wire 116 proximally, which adjusts a curvature of the steering region—e.g. by deflecting the steering region in a first direction. Wire 112 couples plunger 118 to steering region 140, such that sliding of plunger 118 pulls wire 112 proximally, which can also adjust the curvature of the steering region—e.g. by deflecting the steering region in a second direction. The second direction may be opposite to the first direction—e.g. on the same plane. In the example shown, plunger 114 and wire 116 are configured to increase the curvature of the steering region (i.e. to bend the steering region), and plunger 118 and wire 112 are configured to reduce the curvature of the steering region (i.e. to straighten the steering region). However, for some implementations (which are not shown), plunger 118 and wire 112 may be configured to bend the steering region in a different (e.g. opposite) direction to plunger 114 and wire 116.

It is to be noted that, for such implementations, other components of catheter 100 may also contribute to this configuration. For example, steering region 140 itself may be configured to bend only in one direction.

For some implementations, including some implementations in which steering region 140 is configured to bend only in one direction, steering of tube 120 (e.g. through the airways) is facilitated by augmenting bending of the steering region with rotation of the catheter. For example, and as described hereinbelow, steering manipulator 400 may be configured to control steering of steerable region 140 by sliding plungers 114 and 118, and/or by rotating head 110. As shown (FIGS. 5A-C, 6A-B), plungers 114 and 118 may have a circular outer surface (e.g. may be cylindrical) in order to facilitate rotation of head 110 (and thereby catheter 100 as a whole) by steering manipulator 400—e.g. as described in more detail hereinbelow. Head 110 may comprise a head-gearwheel 122 to facilitate such rotation of the head. Head-gearwheel 122 may be fixed to stem 111—e.g. such that axis ax1 is an axis of rotation of the head-gearwheel.

In implementations in which catheter 100 is provided separately from robot 200, the catheter may be a single-use product—e.g. for purposes of hygiene and preventing cross-subject contamination. That is, catheters 100 may be considered to be “consumables” for use with robot 200. For some implementations, other components of system 50 may also be single-use for similar reasons. For example, parts of advancement manipulator 600 that contact tube 120 (e.g. rollers 602 and 604) may also be single-use.

In some implementations, and as shown, e.g. in the exemplary model shown in FIGS. 1 and 2A-B, advancement manipulator 600 may be positioned distally from steering manipulator 400, i.e. closer to subject 10. In some implementations, and as shown in further detail in FIG. 3, manipulator structure 300 may be arranged such that, when catheter 100 is loaded into the robot, head 110 is received by (e.g. is snapped into) steering manipulator 400 (i.e. becomes engaged with the steering manipulator), and tube 120 is received by (e.g. is fed into) advancement manipulator 600.

Thus, as advancement manipulator 600 feeds tube 120 through itself distally toward the body orifice and into the subject, this pulls steering manipulator 400 along advancement path 500, e.g. drawing steering manipulator 400 closer to advancement manipulator 600—thereby shortening the length of the region of tube 120 that is disposed between the two manipulators. That is, as advancement manipulator 600 pushes tube 120 distally, the tube pulls steering manipulator 400 along with it. It is to be noted that, whereas the feeding of the tube through the advancement manipulator is active on the part of the advancement manipulator, the responsive movement of the steering manipulator may be considered to be passive. It is further to be noted that advancement manipulator 600 may remain stationary (e.g. with respect to the body orifice) while catheter 100 and steering manipulator 400 move along the advancement path.

It is to be noted that manipulator assembly 300 therefore advantageously differs from one in which the catheter is advanced by a proximal part of the catheter being pushed distally, and/or in which a unitary device serves both to bend and advance the catheter (e.g. that serves both as a steering manipulator and as an advancement manipulator).

Manipulator assembly 300 may be configured such that advancement manipulator 600 is positionable at least 40 cm (e.g. at least 60 cm, such as at least 80 cm) from steering manipulator 400—i.e. defining a separation between the advancement manipulator and the steering manipulator. For example, such separation may exist before loading of the catheter into the manipulator assembly, and/or after loading but before advancement of the catheter has begun (e.g. when the manipulator assembly is at rest). Steering manipulator 400 may be pullable (by advancement manipulator 600 feeding tube 120) such that the separation becomes less than 20 cm (e.g. less than 10 cm, such as less than 5 cm). Such a reduced separation may occur upon maximal advancement of the catheter. For some implementations, the manipulator assembly may be configured to allow steering manipulator 400 and/or head 110 to reach advancement manipulator 600 (e.g. to be pulled into contact with the advancement manipulator).

As described in more detail hereinbelow, for some implementations, manipulator structure 300 is configured to bias steering manipulators 400 in a proximal direction. For such implementations, the above-described distalward pulling of steering manipulator 400 by advancement manipulator 600 is achieved by the pulling overcoming this bias. Upon advancement manipulator 600 feeding tube 120 proximally (e.g. in order to withdraw the tube from subject 10), or being disengaged from tube 120, steering manipulator 400 responsively moves proximally due to the biasing. Again, this proximalward movement of the steering manipulator is passive on the part of the steering manipulator. Thus, manipulator structure 300 may maintain the region of tube 120 disposed between steering manipulator 400 and advancement manipulator 600 straight (e.g. by maintaining it under nominal tension) during both advancement and retraction of catheter 100. This may advantageously obviate the need for the manipulator structure to include structures that support and/or contact this region of the tube, therefore inter alia increasing hygiene and simplicity of the system. Thus, for some implementations, and as shown, the region of tube 120 disposed (e.g. suspended) between steering manipulator 400 and advancement manipulator 600 is exposed and substantially unsupported. Similarly, for some implementations, and as shown, manipulator structure 300 does not support tube 120 distally from advancement unit 650—i.e. from the part of the manipulator structure that propels the catheter distally.

The described arrangement of manipulator assembly 310 (e.g. steering manipulator 400 and advancement manipulator 600 thereof) enables robot 200 to control both steering (e.g. bending, rotation, and straightening) of steering region 140, and advancement of catheter 100, thereby facilitating maneuvering of catheter 100 into and through a lumen of a subject, such as an airway.

The described arrangement of manipulator assembly 310 also allows advancement manipulator 600 to be stationary and/or positioned close to the point of entry into the subject (e.g. the body orifice, such as the mouth), thereby reducing a length of a free or unsupported portion of the tube 120 that is disposed distally from the advancement manipulator but proximally from the entry point into the subject. Because tube 120 is flexible, reducing the length of this free or unsupported portion of the tube may advantageously reduce undesired or inadvertent buckling of the tube distal to the point at which advancement manipulator 600 pushes the tube distally, thereby increasing the reliability and/or accuracy of the advancement of the tube by the advancement manipulator.

An additional advantage of this described arrangement of manipulator assembly 310 may derive from steering manipulator 400 being spatially and functionally separated from advancement manipulator 600, a feature which allows independent control of steering and catheter advancement.

Once tube 120 has been received by (e.g. loaded into) advancement manipulator 600, operation of the advancement manipulator can pull steering manipulator 400 along the advancement path (e.g. distally and/or toward the advancement manipulator) by feeding the tube though the advancement manipulator (FIG. 3). Thus, each catheter 100 is configured to be loaded into a respective manipulator assembly 310 by (i) loading head 110 into steering manipulator 400, and (ii) loading tube 120 into advancement manipulator 600; and this loading allows the steering manipulator and the advancement manipulator to function as described hereinabove. In some implementations, advancement manipulator 600 is also configured to rotate tube 120, i.e. catheter 100.

Reference is made to FIG. 4, schematically showing a cross-section through advancement unit 650—e.g. a cross-section that is parallel with tubes 120a and 120b. Each advancement manipulator 600 (e.g. 600a, 600b) comprises a set (e.g. a pair) of rollers 602, 604, configured to feed tube 120 (120a, 120b) distally at a rate controllable by the speed of roller rotation. Each set of rollers has adequate opposing force between them to facilitate feeding the tube therethrough, but limited enough to prevent crushing of the tube. The rollers may be cylindrical. In some implementations, one or more of the rollers may have a rough surface, e.g. treads, to facilitate gripping of tube 120. Because rollers 602 and 604 are used to feed tube 120 through the advancement manipulator, they may be termed “feed rollers”.

For some such implementations, the treads may be linear, parallel with each other, and perpendicular with tube 120. Such treads may advantageously grip tube 120 in the direction of linear movement, while nonetheless allowing rotation of the tube—e.g. by rotational slipping of the tube between the rollers. The configuration of the rollers may thus be configured to facilitate both forward/backward movement of catheter 100 as well as rotation thereof.

Reference is now made to FIGS. 5A-C and 6A-B, which are schematic illustrations of an exemplary catheter 100, in accordance with some implementations. Catheter 100 comprises tube 120, having a steering region 140 at a distal portion thereof (not shown in FIGS. 5A-C). Catheter 100 further comprises a first wire 116 and a second wire 112, each wire extending from steering region 140 proximally along tube 120. For some implementations, catheter 100 is configured such that first wire 116 is a bending wire and second wire 112 is a straightening wire. Catheter 100 further comprises a head 110, the head comprising a stem 111, a first plunger 114, and a second plunger 118.

Plunger 114 is operatively coupled to steering region 140 by being attached to wire 116. Plunger 114 is mounted on stem 111 to be slidable linearly along the stem. Plunger 118 is operatively coupled to steering region 140 by being attached to wire 112. Plunger 118 is mounted on stem 111 to be slidable linearly along the stem-typically independently of plunger 114.

Plungers 114, 118 and stem 111 may be complementarily shaped to rotationally lock the plungers to the stem. The complementary shapes may define, e.g. a keyed joint. For example, stem 111 may have a noncircular (e.g. square) outer cross-section, and plungers 114, 118, may have a noncircular (e.g. square) inner cross-section, an arrangement that rotationally locks plungers 114, 118 to stem 111 and facilitates rotation of head 110 independently of axial movement of the plunger.

As shown, wires 116 and 112 pass, from their respective plungers, substantially linearly into tube 120 via holes 106 and 102, from where the wires extend along the tube to steering region 140.

Stem 111 may be shaped to define slots 113 and 117 via which wires 116 and 112, respectively, extend from their respective plungers to tube 120. As shown, slots 113 and 117 may be disposed on opposite sides of stem 111. As shown, slots 113 and 117 may extend along nonidentical axial portions of stem 111—e.g. with at least part of slot 117 extending further proximally than slot 113, and/or with at least part of slot 113 extending further distally than slot 117. However, also as shown, part of slot 113 may be axially coincident with part of slot 117.

For some implementations, slots 113 and 117 may limit the extent of the axial sliding of plungers 114 and 118. For example, each plunger may comprise or be coupled to a respective protrusion that protrudes into the respective slot.

For each plunger, as the plunger slides, the respective wire is pulled by the same amount in the same direction.

Stem 111 may be fixedly connected to head-gearwheel 122, which is configured to engage a manipulator-gearwheel 422 on steering manipulator 400, as further described hereinbelow. Catheter 100 typically further comprises a port 130, in fluid communication with tube 120, and via which a variety of medical instruments and imaging tools (e.g. a camera, an ultrasound probe, or a medical tool such as a biopsy needle) may be advanced into and through the tube.

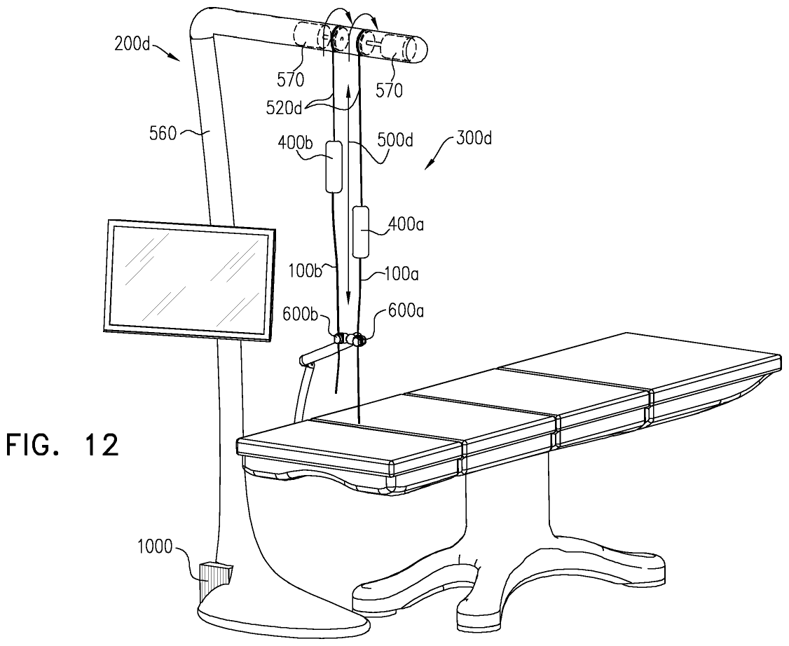

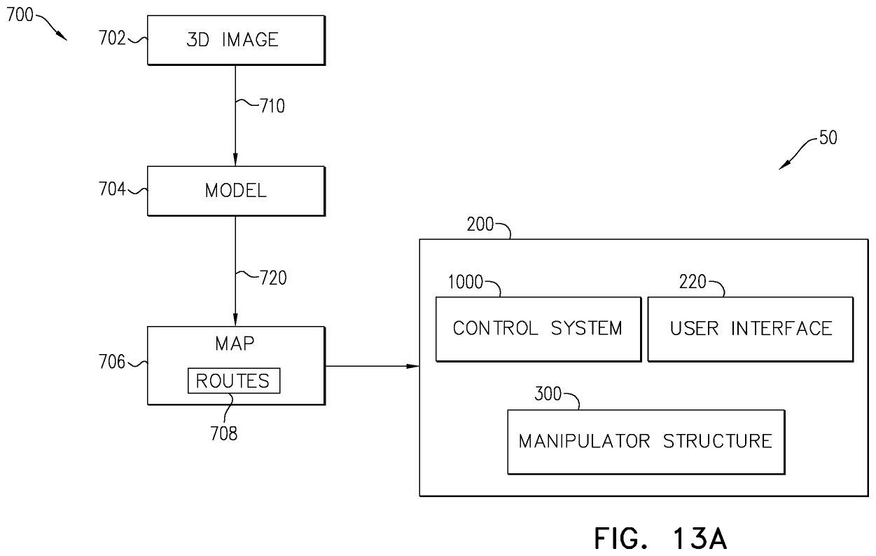

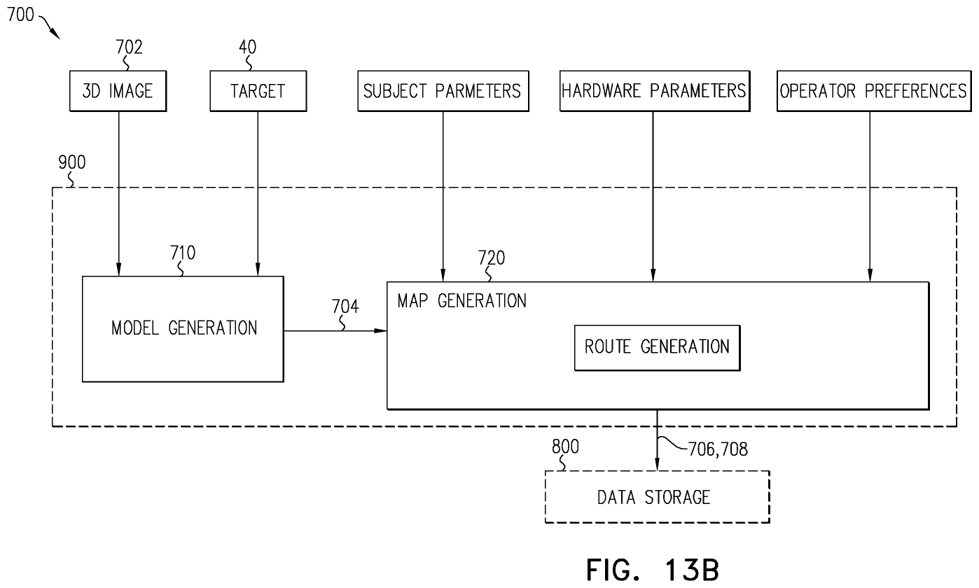

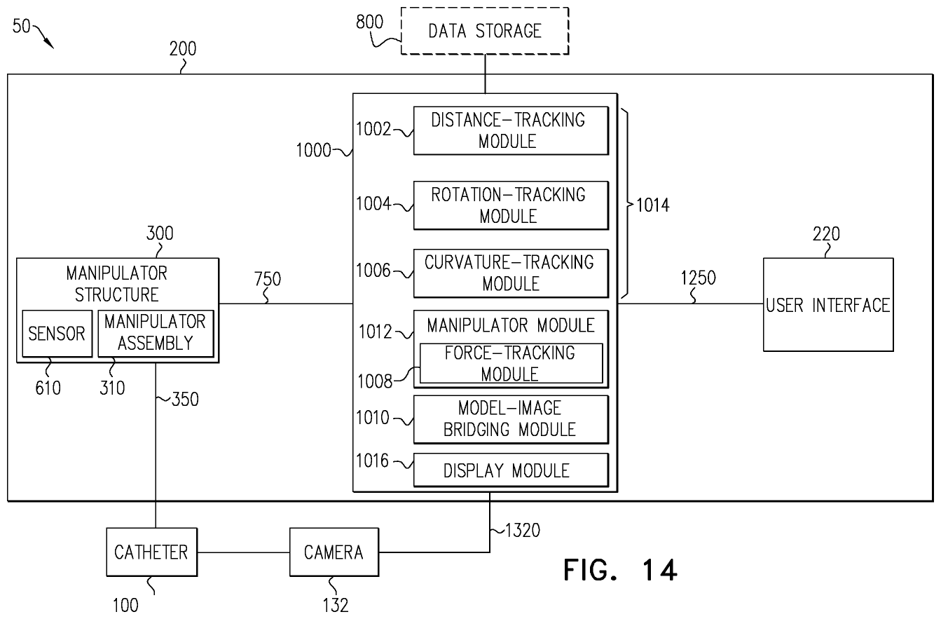

For some implementations, catheter 100 (e.g. each catheter 100) is advanced with a camera 132 (FIG. 2B) already disposed at the distal end of the catheter (e.g. extending through the catheter to the distal end of the catheter), providing imaging data (i.e. an image input) that can be used (e.g. by system 1000) to facilitate guidance of the catheter along the airways (e.g. tracing of the catheter along a planned route through the airways). For some implementations, this may be as described in International Patent Implementation PCT/IB2022/057505 to Shapira et al. which is incorporated herein by reference. This subject is described in more detail hereinbelow—e.g. with reference to FIGS. 16A-C.

It is to be noted that, due to the above-described configuration of catheter 100 (e.g. of head 110), no stress is storable in the catheter itself independently of an externally-applied force. Therefore, any curvature conferred on steering region 140 by head 110 (via wires 112 and 116), is (e.g. must be) maintained by steering manipulator 400, as further described hereinbelow.

A safety feature advantageously derived from this lack of stress storable in the catheter, is that a medical procedure may be aborted rapidly simply by disengaging head 110 from steering manipulator 400. As a result, any curvature conferred on steering region 140 by head 110 ceases to be maintained, and the steering region may therefore be withdrawn from the subject, as it passively complies with curvature of the anatomy. This feature is in contrast to, for example, a knob-based pullwire system in which, in any given rotational position of the knob, the knob would maintain a respective curvature of the steering region.

As described hereinbelow, for some implementations, catheter 100 is configured to allow steering region 140 to become stiffened on demand, e.g. via the engagement of head 110 by steering manipulator 400. For such implementations, disengagement of head 110 from steering manipulator 400 would result in steering region 140 immediately becoming limp-which further facilitates rapid abortion of a procedure.

A further advantageous consequence of the design of catheter 100 is the geometric relationship between each plunger, the wire to which it is attached, and the steering region. Linear movement of each plunger translates into an equivalent amount of movement of the wire, which translates into a known amount of curvature of steering region 140. For example, in the position of plungers 114 and 118 shown in FIG. 4A steering region 140 is straight, whereas in FIG. 4B plunger 114 has been slid proximally, pulling wire 116 proximally and increasing the curvature of steering region 140 as the length of the wire within the steering region decreases. Concurrently, plunger 118 has been slid distally, in order to allow the length of wire 112 within steering region 140 to increase in order to accommodate the increased curvature of the steering region.

In some implementations, within steering region 140, wires 116 and 112 may be arranged in a force-multiplication arrangement, as further described hereinbelow. In such arrangements, the geometric relationship between plungers 114, 118, wires 116, 112, and steering region 140 may be multiplied or divided, depending on the particulars of the force-multiplication arrangement.

In some implementations, and as shown, at least steering region 140 of tube 120 may comprise a series of vertebrae 144, arranged in a chain and having a predetermined compressive strength. Vertebrae 144 may overlap and/or intercalate with each other. Vertebrae 144 may facilitate controlled bending of steering region 140—e.g. by confining the steering region to a limited number of bending planes, by inhibiting axial compression of the steering region in response to tensioning of wires 112 and 116, and/or by defining a minimum radius of curvature of the steering region. Furthermore, vertebrae 144 may be configured to contribute to stiffening of steering region 140 in response to balanced tensioning of both wires 112 and 116—e.g. as described in more detail hereinbelow with reference to FIGS. 9A-H.

Reference is now made to FIGS. 7A-D, which are schematic illustrations of steering manipulator 400 viewed from various perspectives, in accordance with some implementations. Steering manipulator 400 comprises a housing 480 that houses at least one control unit, and in some implementations and as shown, two control units 402, 404. Each control unit 402, 404 is controllable independently of the other, although, as described hereinbelow, robot 200 (e.g. robotic control system 1000 thereof) is typically configured to coordinate both control units of a given steering manipulator 400 so as to coordinate the axial position of both plungers of the head 110 of a corresponding catheter 100, in order to control the steering region of that catheter.

The following description applies to both control unit 402 and control unit 404, except where noted. Because of the similarity between the components and functionality of the control units, pairs of reference numerals (e.g. 402, 404) are used throughout the description of steering manipulator 400, in order to indicate that a particular point applies to the component of both control units. Furthermore, despite the similarity between control units 402 and 404, they and/or components thereof may not be shaped identically or as mirror images of each other, e.g. in order to interact with different components (e.g. plungers) of head 110 of catheter 100.

Each control unit 402, 404 comprises an actuator 410, 430, a spring 412, 432, and a push-face 419, 439. In the implementation shown, each push-face is defined by a respective cradle 418, 438. However, it is to be noted that, for some implementations, such a push-face may be provided by a structure other than a cradle. In some implementations, cradle 418, 438 comprises or defines an abutment 416, 436. Actuator 410, 430 is operatively coupled to the cradle via the spring, such that the spring can transfer force from the actuator to the cradle. Spring 412, 432 is calibrated to a known spring constant. Spring 412, 432 may be a compression spring and/or may be disposed axially between the actuator and the cradle—e.g. as shown. Cradle 418 is configured (e.g. dimensioned) to receive plunger 118, and cradle 438 is configured to receive plunger 114 (e.g. as described hereinbelow with reference to FIGS. 8A-C). Actuator 410, 430 is configured to be driven linearly along a respective threaded rod 417, 437—e.g. by a motor 450, 460 that rotates the rod.

As noted, spring 412, 432 may be a compression spring. However, the scope of the present disclosure includes other classes of spring including, but not limited to, a tension spring and a torsion spring. Furthermore, although spring 412, 432 is shown as a coil spring, the scope of the present disclosure includes other types of spring including, but not limited to, a cantilever spring, a gas spring, and a resilient material such as a rubber.

Control units 402, 404 are configured to move plungers 114, 118 axially independently of each other and independently of (e.g. without) moving stem 111.

As shown, at least one support rod 413, 433 may be provided to support and/or stabilize each control unit—e.g. the actuator, the spring, and/or the abutment may be slidable along the support rod, which may pass through one or more of these components—e.g. as shown. Driving the actuator toward the abutment (by motor 450, 460 rotating rod 417, 437) applies a force on spring 412, 432, which transfers the force to abutment 416, 436, thereby pushing cradle 418, 438—e.g. in the same direction as the actuator. When head 110 is loaded in steering manipulator 400, this pushing of the cradle slides the corresponding plunger 118, 114 along stem 111—e.g. by pushing the respective push-face 419, 439 proximally against the plunger.

Control unit 402, 404 comprises an actuator encoder 414, 434 and a cradle encoder 415, 435 (FIGS. 7A-B). Actuator encoder 414, 434 is configured to provide an output (e.g. an “actuator output”) indicative of a linear position of actuator 410, 430—e.g. with respect to housing 480. Cradle encoder 434, 435 is configured to provide an output (e.g. a “cradle output”) indicative of a linear position of cradle 416, 436—e.g. with respect to housing 480 or another reference point. Thus, the actuator encoder and the cradle encoder are linear encoders. The actuator encoder and the cradle encoder may each comprise a readhead paired with a scale. For example, the scale may be fixedly attached to housing 480 while the sensor is fixedly attached to the actuator or the cradle, respectively. The outputs of the encoders are typically electronic, and may be delivered to robotic control system 1000—e.g. electrically or wirelessly.

Control system 1000 may be configured to determine the tension magnitude in the corresponding wire of a given control unit responsively to the actuator output, the cradle output, and the predetermined spring constant. Furthermore, control system 1000 may be configured to send output to manipulator assembly 310, e.g. to steering manipulator 400, to prevent the tension magnitude in the corresponding wire from rising above a predetermined level, and/or to warn an operator about the tension magnitude in the corresponding wire rising above a predetermined level.

Each control unit is configured such that a distance between the actuator and the cradle is dependent on resistance of the cradle to being pushed via the spring. As described hereinbelow, once head 110 of catheter 100 is loaded into steering manipulator 400, such resistance may be provided by tension in the wire that is attached to the plunger that is in its cradle. Thus, the tension in the wire is determinable by robotic control system 1000 responsively to the outputs of the encoders. Such tension in the wire may, in turn, be the result of resistance to deflection (e.g. bending or straightening) of steering region 140. The control unit is configured to act in a closed loop to automatically adjust tension in response to sensing a change in a position of the plunger.

Although steering region 140 itself may not materially resist deflection, resistance may be experienced when the steering region is disposed within or adjacent a tissue—e.g. as described in more detail elsewhere herein.

In addition to motor 450 and motor 460, steering manipulator 400 may comprise a third motor 440 configured to rotate head 110 of catheter 100 (FIG. 7D). Catheter 100 is typically configured such that rotation of head 110 also rotates tube 120—i.e. rotates the entire catheter. In the example shown, motor 440 rotates manipulator-gearwheel 442, which is complementary to head-gearwheel 122 on head 110, thereby rotating head 110. In implementations in which rotation of head 110 and bending/straightening of steering region 140 are controlled by a series of motors, in this way bending, straightening, and rotation of steering region 140 may thus be coordinated (e.g. in concert and/or independently) by manipulation of head 110 by steering manipulator 400.

Reference is now made to FIGS. 8A-C, which are schematic illustrations of steering manipulator 400, head 110 of catheter 100, and interactions therebetween, in accordance with some implementations. FIGS. 8A-B show head 110 being introduced into steering manipulator 400 while at least one gate 448 of the steering manipulator (e.g. of housing 480 thereof) is open. FIG. 8C shows gate 448 being closed to lock head 110 in place.

Steering manipulator 400 is configured to receive head 110 in a manner that enables locking head 110 into steering manipulator 400, thereby operatively coupling the steering manipulator to the head, and thereby to steering region 140. When head 110 is engaged within steering manipulator 400, plunger 114 is fitted within (e.g. cradled by) cradle 418, and plunger 118 is fitted within (e.g. cradled by) cradle 438.

Thus, once head 110 has been received by (e.g. loaded into) steering manipulator 400, steering manipulator 400 is configured to manipulate steering region 140 (e.g. a curvature thereof) by controlling sliding of plungers 118, 114 linearly along the stem.

As noted above, system 50 may be configured such that the engagement of head 110 by steering manipulator 400 enables measurement and calibration of tension on each of wires 116 and 112, via robotic control system 1000. This is described in more detail hereinbelow. The force being applied to each spring is important both for controlling steering region 140 of tube 120 and also to provide feedback from steering region 140 regarding force being applied on a distal region of tube 120 by the anatomy, e.g. a lumen, through which tube 120 is being maneuvered. The force is determined by robotic control system 1000 based on position information provided by the respective actuator and cradle encoders.

Steering manipulator 400 may comprise at least one gate 448—e.g. two gates, one at each end of housing 480. Gate 448 may comprise (i) a closure 444 configured to maintain the engagement of head 110 by the steering manipulator, and/or (ii) a roller 446 configured to facilitate rotation of head 110 (and thereby of catheter 100) while maintaining engagement between head 110 and steering manipulator 400. To further facilitate the rotatability of head 110 within steering manipulator 400, the head may have one or more circular surfaces (e.g. circumscribing head axis ax1) over which roller 446 may ride. In the example shown, head 110 has a distal such circular surface 121 (which may be defined by a connector that connects stem 111 to tube 120), and a proximal such circular surface 123, which may be adjacent to port 130, may be defined by port 130, or may connect port 130 to stem 111. As noted above, stem 111 itself may nonetheless be noncircular—e.g. may be square in cross-section.

Gear 122 on head 110 is configured such that, when the head is inserted into steering manipulator 400, gear 122 enmeshes with manipulator-gearwheel 422 of the steering manipulator, linked to motor 440 configured to rotate head 110, as further explained hereinbelow. Plungers 114, 118 are advantageously rotatable in their respective cradles, i.e. each plunger is configured to slip rotationally within its cradle, without impinging on the ability of the cradles to linearly move the plunger, e.g. without locking the plungers in place axially. Thus, each plunger may be moved axially in both distal and proximal directions while concurrently being rotated.

In some implementations, and as shown, gate 448 (444, 446) comprises a clamp, e.g. a latch clamp, toggle clamp, or snap-lock mechanism. However, other gate or locking mechanisms are also contemplated. Furthermore, for some implementations steering manipulator 400 may not include a discrete gate at all, but rather head 110 may be merely snap-fitted into the steering manipulator.

Reference is now made to FIGS. 9A-H, which are schematic illustrations showing steering manipulator 400 manipulating head 110, in accordance with some implementations. Briefly: FIG. 9A shows a state immediately following insertion of head 110 into steering manipulator 400; FIG. 9B shows a state after a calibration step; FIG. 9C shows a state after a stiffening step; FIG. 9D shows a state as steering region 140 is bent; FIGS. 9E-F show closed-loop feedback to maintain the curvature of steering region 140 under conditions in which an external force is applied thereto; FIG. 9G shows a state as steering region 140 is stiffened while retaining its bent curvature; and FIG. 9H shows a state as catheter 100 is rotated while the steering region remains stiffened and bent. Despite the apparent stepwise nature of FIGS. 9A-H, and although these figures may actually represent discrete steps according to some implementations, it is nonetheless to be understood that these figures are primarily intended to demonstrate functionality of the steering manipulator and the catheter.

In each of FIGS. 9A-H, double arrow A1, A2 signifies the linear position of cradle 438, 418 within housing 480, and double arrow B1, B2 signifies the linear position of linear actuator 410, 430 within the housing. In the figures, the linear position of the cradle is arbitrarily represented as the distance between the proximal end of housing 480 and the distal end of abutment 416, 436, which is axially fixed with respect to the cradle and the plunger cradled therewithin. In the figures, the linear position of the linear actuator within the housing is arbitrarily represented as the distance between the proximal end of the housing and the distal end of the linear actuator. Double arrow H1, H2 represents a distance between the linear actuator 410, 430 and the abutment 416, 436—which varies with the length of spring 412, 432.

A1, A2, B1, B2, H1, and H2 change between the figures, thus indicating a change in position of a component of the respective control unit.

The white, single-headed arrows along the linear actuators, abutments, and plungers indicate a direction of linear movement of that component with respect to housing 480. In each figure, a corresponding state of steering region 140 is also shown.

Although robotic control system 1000 is not shown in FIGS. 9A-H, the operation of control units 402 and 404 described with reference to these figures is typically performed by the robotic control system, which is in electronic communication with the control units. The robotic control system may use algorithms stored therewithin to measure and equalize the forces applied to each spring, and thus to each plunger, as further described hereinbelow.

In FIGS. 9A-H, and the descriptions thereof, wire 116 is expressly referred to as a bending wire (i.e. pulling of wire 116 increases but cannot reduce the curvature of steering region 140), and wire 112 is expressly referred to as a straightening wire (i.e. pulling of wire 112 decreases but cannot increase the curvature of steering region 140). However, and as described elsewhere herein, it is to be understood that for some implementations wires 112 and 116 may both be configured to bend steering region 140, although in different (e.g. opposite) directions.

In FIG. 9A, head 110 is shown having been loaded into, and engaged by, steering manipulator 400—e.g. as described hereinabove. Plunger 118 rests in cradle 418, and plunger 114 rests in cradle 438. At this point, the linear position of the cradles (and thereby those of the plungers) may be such that bending wire 116 and/or straightening wire 112 are slack (e.g. see inset of FIG. 9A). Moreover, for some implementations the cradles may not be in any particular (e.g. pre-defined) linear position. However, for some implementations, steering manipulator 400 is provided (e.g. by control system 1000) with a loading state in which the cradles are positioned sufficiently distally that the plungers can be inserted into the cradles without tensioning the wires.

FIG. 9B shows the slack on wires having been taken up (e.g. eliminated) by implementation of a sliding force to the plunger, i.e. via sliding linear actuators 410 and 430 proximally, such that, via the corresponding springs, the abutments, the cradles, and plungers 118 and 114 are pushed proximally. In the example shown, the linear actuators are slid until a point at which control system 1000 determines that the slack has been eliminated. In the example shown, this is achieved by system 1000 identifying a reduction in distance H1, H2. For example, until the slack has been eliminated, distance H1, H2 may remain constant whereas once the slack has been eliminated, the distance begins to reduce. For some implementations, system 1000 may determine a precise position in which the wires have zero slack and zero tension. For some implementations, this is achieved by sliding the linear actuators back and forth to identify this precise position, distal to which distance H1, H2 remains constant but proximal to which the distance changes with changes in the position of the linear actuator.

The transition from FIG. 9A to FIG. 9B may therefore represent a calibration step (e.g. FIG. 9B may represent a calibrated state). Such calibration may be performed automatically by system 1000—e.g. upon loading of head 110 into steering manipulator 400, and/or upon an input from the operator. Such automated calibration for each catheter 100 may advantageously facilitate efficient and/or cost-effective manufacture of catheter 100. For example, each catheter 100 may be manufactured with relatively high tolerance with respect to the length of wires 112 and 116, and/or may not require precise factory calibration. Rather, robot 200 calibrates to each particular catheter that is loaded.

Although the distance by which linear actuators 410 and 430 move proximally between FIGS. 9A and 9B may appear similar, these distances are not necessarily equal, as each plunger and wire are calibrated independently. The absolute axial position of each control unit at a point of calibration may vary along the longitudinal axis of the steering manipulator. Thus, distances A1 and A2 at a point of calibration may vary, depending on the position of abutments 416 and 436, respectively, for a given catheter.

As noted hereinabove, catheter 100 may be configured such that steering region 140 is stiffenable by tensioning both wire 112 and wire 116—e.g. in a balanced manner. This balanced tensioning may, for example, involve applying the same amount of tension to both wires. However, and as noted hereinbelow, for some implementations stiffening may be achieved with different tension on each wire. Tension applied to wire 112 may inhibit steering region 140 from passively deflecting in a first direction in response to an exogenous force (e.g. from contact with tissue), and tension applied to wire 116 may similarly inhibit passive deflection in the opposite direction.

For some implementations, for operation of catheter 100 (e.g. for advancement and bending of steering region 140), conferring a certain (e.g. a baseline) degree of stiffness on steering region 140 may be advantageous—e.g. so that the steering region resists passive bending while being pushed through the anatomy (e.g. pushed through the airways). Thus, applying a certain level of tension on both wires may be advantageous in order to provide such stiffness. The stiffness may result from the opposing forces of the two wires, and/or from the behavior of vertebrae 144 in response thereto. The transition from FIG. 9B to FIG. 9C illustrates such conferring of stiffness.

In FIG. 9C, steering manipulator 400 and head 110 are shown after steering region 140 has been stiffened by tensioning both wires 112 and 116. In the particular example shown, steering region 140 has been stiffened while straight—e.g. without any change in the curvature of the steering region. Thus, compared with FIG. 9B, both linear actuators 410 and 430 have moved proximally, but because of the balanced nature of the tensioning of the wires, plungers 118 and 114 move less than the linear actuators—and may even not move at all. This is schematically represented in the figures by the plungers not having moved.

Using the outputs from the actuator encoders and the cradle encoders (e.g. the distance moved from the calibrated state), along with the known spring constant of each spring, control system 1000 is enabled to determine the tension on each wire. Thus, control system 1000 can, by moving the linear actuators, confer stiffness on steering region 140 by balancing tension in wires 112 and 116—e.g. by concurrently increasing the tension in both wires in a balanced manner.

For some implementations, the degree of stiffness of steering region 140 may be predetermined (e.g. may be a baseline stiffness, which may be factory-set). For some such implementations, the predetermined degree of stiffness may be specific to a particular type of catheter 100.

For some implementations, the degree of stiffness of steering region 140 is selectable—e.g. by the operator, who may alter the stiffness throughout the course of the procedure.

For some implementations, the degree of stiffness of steering region 140 is dynamically controlled by control system 1000 (e.g. throughout the procedure)—e.g. in response to a pre-planned procedure, and/or in response to detected characteristics of the tissue surrounding the steering region. Tensioning of steering region 140 may be done in a continuous manner, such that the tension in each wire is adjusted continuously and individually, in response to feedback from the distal end, or a priori, e.g. via the robotic control system according to a planned route.

Note that head 110 itself, when not engaged with steering manipulator 400, stores no tension in wires 112 and 116, and therefore does not maintain stiffness in steering region 140. Rather, these effects are a consequence of engagement between head 110 and steering manipulator 400—i.e. when plungers 118, 114 are cradled in cradles 418 and 438 respectively, the plungers transmit force from control units 402, 404, via wires 112, 116, to steering region 140.

It is to be noted that, although FIG. 9B is described as representing a discrete calibrated state, robot 200 may in fact proceed directly (or at least apparently directly) from loading of head 110 to the conferring of stiffness on steering region 140. Similarly, it is to be noted that the stiffening represented by FIG. 9C may also not be a discrete step. Moreover, for some implementations no stiffening is conferred on steering region 140 prior to its advancement into the subject.

In FIG. 9D, steering manipulator 400 and head 110 are shown in positions corresponding to steering region 140 having a degree of curvature. The illustrated position may follow in sequence from either FIG. 9B or 9C, or at any point after head 110 has been fixed in place within steering manipulator 400. The resulting curvature of the steering region in FIG. 9D is achieved by moving plunger 114 proximally (leftward in the figure) along stem 111, thereby shortening the length of wire 116 within steering region 140, and thereby increasing the curvature of the steering region. Typically, and as shown, plunger 118 is concurrently moved distally (rightward in the figure), allowing more of wire 112 to enter into steering region 140 and thereby enabling the steering region to bend in response to the pulling of wire 116. As described hereinabove, the movement of plungers 114 and 118 (which are disposed in their respective cradles) is achieved by moving linear actuators 430 and 410 in the same direction as their respective plungers—which, in this case, means that the linear actuators are moved in opposite directions to each other.

In order to maintain the stiffness in steering region 140 as the steering region is bending, control system 1000 may operate control units 404 and 402 to move plungers 114 and 118 while monitoring and coordinating distances H1 and H2—and thereby maintaining tension on wires 116 and 112. For some implementations, such coordination involves maintaining distances H1 and H2 constant. However, for some implementations, distances H1 and H2 may change despite the stiffness of steering region 140 being maintained.

For some implementations, such coordination of distances H1 and H2 is achieved by moving the plungers by the same distance-albeit in opposite directions. However, for some implementations, the distances by which the plungers move may differ from each other, even significantly, and/or even if distances H1 and H2 do not change. For example, for implementations in which a force-multiplication system (e.g. a pulley arrangement) is used to provide mechanical advantage to one or both of wires 116 and 112 within the steering region, the distance by which one plunger (e.g. plunger 114) moves may be a multiple of the distance by which the other plunger (e.g. plunger 118) moves. (Utilization of such force-multiplication, and the effects and advantages that it may confer on the functionality of system 50, are described in more detail hereinbelow.) Thus, the stiffness of steering region 140 is controllable independently from the curvature thereof.

FIGS. 9E-F illustrate how control unit 402, 404 may be configured to act in a closed loop with control system 1000 to automatically maintain a configuration (e.g. a curvature) of steering region 140, e.g. that shown in FIG. 9D. Typically, during the use of system 50, the curvature of steering region 140 is repeatedly adjusted into a curvature that is desired for the present stage of the procedure (e.g. to navigate a particular anatomical feature). The curvature shown in FIGS. 9D-F is an example of a particular such “desired curvature” that may be desired for a specific stage of a given procedure.

During the use of system 50, an external force may act on steering region 140 in a manner that undesirably alters the curvature of the steering region away from the desired curvature—or at least that may do so in the absence of the presently-described closed loop. For example, such forces may be exerted by a wall of an anatomical lumen through which steering region 140 is passing, or by a tool moving through tube 120. Such a force (represented in FIGS. 9E-F by arrows 146 and 148) would naturally be transferred via wires 116, 112 to plungers 114, 118.

For example, and with reference to FIG. 9E, force 146 has a vector that would reduce the curvature of steering region 140. This may be caused, for example, by a tool 138 being advanced through the steering region, the presence of the tool urging the steering region to straighten. This reduction in curvature would necessarily urge plunger 114 distally by pulling on wire 116. Control system 1000 is configured to immediately sense, via encoder 435, such distalward movement as distalward movement of cradle 438, and to responsively (e.g. instantaneously) return the cradle (and thereby the plunger) proximally, thereby restoring the desired curvature of steering region 140. Thus, in FIG. 9E, plunger 114 (in cradle 438) is in the same position (i.e. has been returned to the same position) as that in FIG. 9D, which is the position that corresponds to the desired curvature of steering region 140. That is, distance A2 is identical in FIGS. 9D and 9E. This repositioning is achieved by moving linear actuator 430 proximally, thereby pushing abutment 436 proximally. As described hereinabove, this pushing is applied via spring 432, which compresses (i.e. H2 decreases) responsively to the opposition between force 146 and the pushing of linear actuator 430.

FIG. 9F shows another example, in which a force 148, applied to steering region 140, has a vector that would increase the curvature of steering region 140. This increase in curvature would necessarily urge plunger 118 distally by pulling on wire 112. Control system 1000 is configured to immediately sense, via encoder 415, such distalward movement as distalward movement of cradle 418, and to responsively (e.g. instantaneously) return the cradle (and thereby the plunger) proximally, thereby restoring the desired curvature of steering region 140. Thus, in FIG. 9F, plunger 118 (in cradle 418) is in the same position (i.e. has been returned to the same position) as that in FIG. 9D, which is the position that corresponds to the desired curvature of steering region 140. That is, distance A1 is identical in FIGS. 9D and 9F. This repositioning is achieved by moving linear actuator 410 proximally, thereby pushing abutment 416 proximally. As described hereinabove, this pushing is applied via spring 412, which compresses (i.e. H1 decreases) responsively to the opposition between force 148 and the pushing of linear actuator 410.

It is to be noted that control system 1000 can therefore indirectly determine forces 146 and 148 by virtue of (i) the known spring constants of springs 432 and 412, and (ii) deriving distances H2 and H1 (e.g. changes thereto) from the outputs of encoders 434 and 435 and encoders 414 and 415. This is therefore indicative of the tension applied to wires 116 and 112 in order to overcome these forces. This may advantageously allow control system 1000 and/or the user to monitor and/or limit the forces applied by robot 200 and the tension in the wires.

In summary, control system 1000 is configured to maintain the curvature of steering region 140 by moving the linear actuators as needed to adjust distances H1 and H2, in order to maintain plungers 114 and 118 in their desired position, such that distances A1 and A2 are maintained irrespective of advancement and manipulation of tools within the steering region.

FIG. 9G illustrates a manner in which steering manipulator 400 may be configured to stiffen steering region 140. In the example shown, the curvature is the same as that in FIG. 9D—e.g. FIG. 9G may be considered to show a step that follows, e.g. after the state illustrated in FIG. 9D. In FIG. 9G, the stiffness of steering region is greatly increased—e.g. the curvature of the steering region may, in effect, be locked. The stiffening is accomplished similarly to the stiffening shown in FIG. 9C, i.e. by concurrently compressing each spring 412, 432 by moving the respective linear actuator 410, 430 proximally, thereby compressing the spring between the linear actuator and the respective abutment 416, 436. Plungers 114 and 118, in their respective cradles, may move very little—or may not move at all—with respect to each other and/or with respect to housing 480. That is, distances A1 and A2 may not decrease significantly—or even at all. For some implementations, this stiffening (e.g. locking) is augmented by vertebrae 144—e.g. by interactions between adjacent vertebrae, e.g. resulting from the tension in wires 112 and 116 axially compressing steering region 140. It is to be noted that system 50 thereby advantageously facilitates stiffening of steering region 140 while retaining the same curvature thereof. This may, for example, allow steering region 140 to be positioned and angled appropriately for accessing a particular target within the body, and then subsequently advancing a tool through tube 120 without inadvertently changing the position or angle of the distal tip of the tube.

In FIG. 9H, catheter 100 is shown as having been rotated by about 90 degrees from the position shown in FIG. 9G. Rotation of head 110 is performed by rotation of manipulator-gearwheel 442 of steering manipulator 400, which is enmeshed with head-gearwheel 122 of head 110, thereby rotating head 110. Manipulator-gearwheel 442 is not visible in FIGS. 9A-F. Rotation head 110 is facilitated by plunger 118 passively rotating within cradle 418, and plunger 114 passively rotating within cradle 438. Rotation of catheter 100 does not require or cause any of the components of control unit 402 or 404 to move, so the stiffness of the steering region and its angle of curvature can be maintained independently of the rotation.

It is to be noted that each of the illustrated positions and actions illustrated in FIGS. 9A-H are shown in isolation. Under conditions of use in an endoscopic human procedure, the steering region may be stiffened, bent, and rotated as a continuous sequence and/or some actions may be performed simultaneously. Robotic control system 1000 maintains tension on the steering region according to predetermined limits and in some implementations, as defined by the operator prior to or during the procedure.

Reference is again made to FIGS. 7A-9H. For some implementations, the distance between a cradle and its corresponding linear actuator (e.g. distance H1 or H2) may be measured more directly than as described hereinabove. For some implementations, a linear encoder may include a movable scale fixed with respect to the actuator or with respect to the cradle. For example, such a linear encoder may include a movable scale fixed with respect to the actuator, and a readhead fixed with respect to the cradle. Similarly, such a linear encoder may include a movable scale fixed with respect to the cradle, and a readhead fixed with respect to the actuator. In these examples, the linear encoder that includes the movable scale may provide an output that is more directly indicative of distance H1 or H2. Nonetheless, these examples typically also include a cradle encoder—e.g. for reasons described elsewhere herein.

In some implementations, control unit 402 and/or 404 comprise a force sensor (e.g. a strain gauge) coupled functionally between the linear actuator and the cradle or its abutment. The inclusion of a force sensor may obviate the need for the actuator encoder and/or for determining the distance between the cradle and the linear actuator. For such implementations, a cradle encoder is nonetheless typically included in the control unit—e.g. for reasons described elsewhere herein.

Components 418 and 438 are referred to hereinabove as cradles, and are illustrated and described as such—e.g. as receptacles that receive the plungers of catheter head 110. However, for some implementations, these components may be simplified to simple projections that are shaped and positioned to apply force to their respective plungers. For example, component 418 may comprise a projection that resides just distal to plunger 118, and component 438 may comprise a projection that resides just distal to plunger 114, such that proximal movement of the projection pushes the respective plunger proximally, thereby pulling on the respective wire. Such a configuration may advantageously facilitate loading of the catheter head into the steering manipulator, and/or may facilitate cleaning and decontamination of the steering manipulator between procedures.

Reference is now made to FIGS. 10A-B, which are schematic illustrations of a sensor 620 for detecting advancement and/or rotation of tube 120, in accordance with some implementations. Sensor 620 may be considered to be a variant of sensor 610, and/or may be used in place of sensor 610 described hereinabove. Sensor 620 may comprise a rider (e.g. a rider roller) 622 and a reader 630. Reader 630 may comprise, e.g. an optical reader that may comprise a light source (e.g. an LED) and a light detector. In some implementations, sensor 620 (e.g. reader 630 thereof) may comprise an electromechanical encoder.

Rider 622 may be a substantially cylindrical roller—e.g. rolling responsively to axial movement of tube 120. Alternatively, and as shown, rider 622 may be spherical—e.g. rolling responsively to axial and rotational movement of tube 120.

Sensor 620 may be disposed downstream of advancement manipulator 600. Sensor 620 is typically resiliently mounted (e.g. to advancement manipulator 600) in a manner that maintains contact between rider 622 and tube 120, such that rider 622 rolls responsively to movement of the tube. In some implementations, sensor 620 is coupled to advancement manipulator 620 via a spring 628, such that rider 622 rests on tube 120 and maintains contact therewith as tube 120 is advanced and/or rotated. Such an arrangement is configured to maintain fidelity of the contact between rider 622 and tube 120, regardless of deviations in movement of tube 120 from a precise linear path and/or variations in the diameter of tube 120. That is, sensor 620 can move responsively to tube 120. In contrast, sensor 620 typically holds reader 630 at a fixed position (e.g. distance) from rider 622.

In some implementations, and as shown, spherical rider 622 is configured to roll responsively to movement of tube 120, such that reader 630 measures movement of rider 622 in both x and y dimensions, corresponding to both advancement and rotation of tube 120. Sensor 620 may be used to verify a position of tube 120, i.e. movement both linearly and rotationally, from a zero or initial calibration point. In some implementations, sensor 620 may be used as a primary means to provide a precise measurement of the distance tube 120 travels.

Such verification may be advantageous for implementations in which rollers 602 and 604 are allowed some degree of slippage (e.g. the rollers may have limited gripping to allow rotation of tube 120, and/or limited clamping to avoid crushing the tube), and/or for implementations in which rotation of head 110 may be imperfectly transferred by tube 120 to steering region 140.

It may be particularly advantageous to obtain such verification toward a distal part of robot 200—e.g. close to the point of entry into the subject—as this may increase accuracy in determining the distance by which steering region 140 has been advanced into the subject, and/or has been rotated.

In some implementations, and as shown in FIG. 10B, rollers 602 and 604 of advancement manipulator 600 may be cylindrical rollers. Such rollers may be provided as part of advancement manipulator 600 and/or may be provided as a separate, consumable component intended for a single use. The provision of rollers 602, 604 as individual-use components aids in prevention of contamination of advancement manipulator 600 by secretions of the subject that may adhere to tube 120 as it is withdrawn proximally. Similarly, rider 622, and/or sensor 620 as a whole, may be a single-use component.

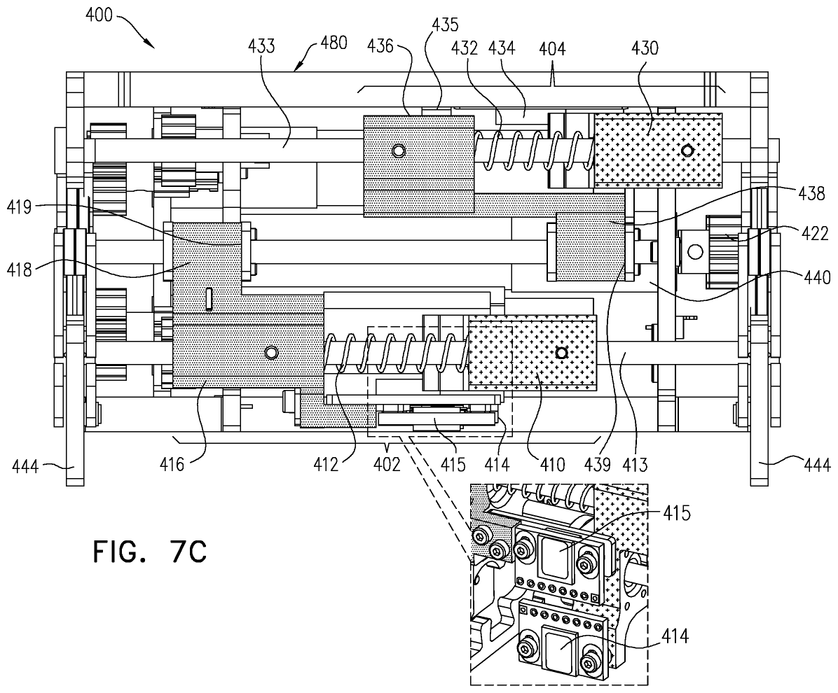

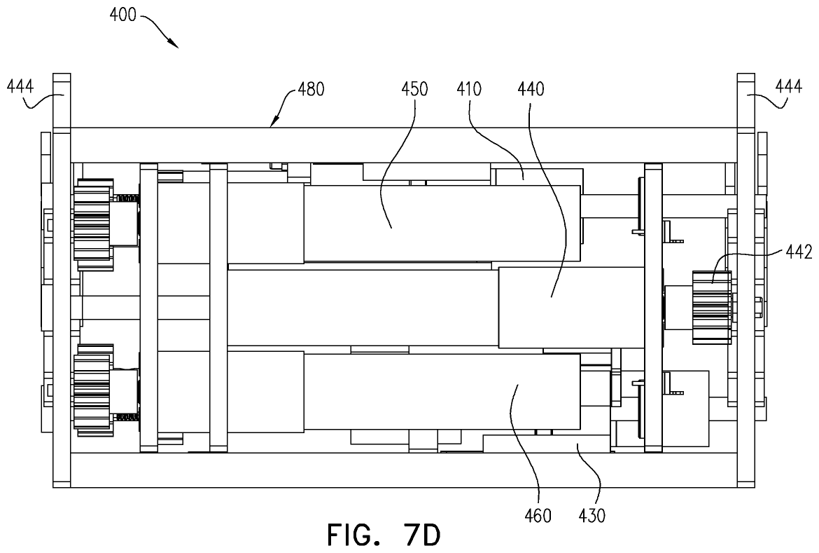

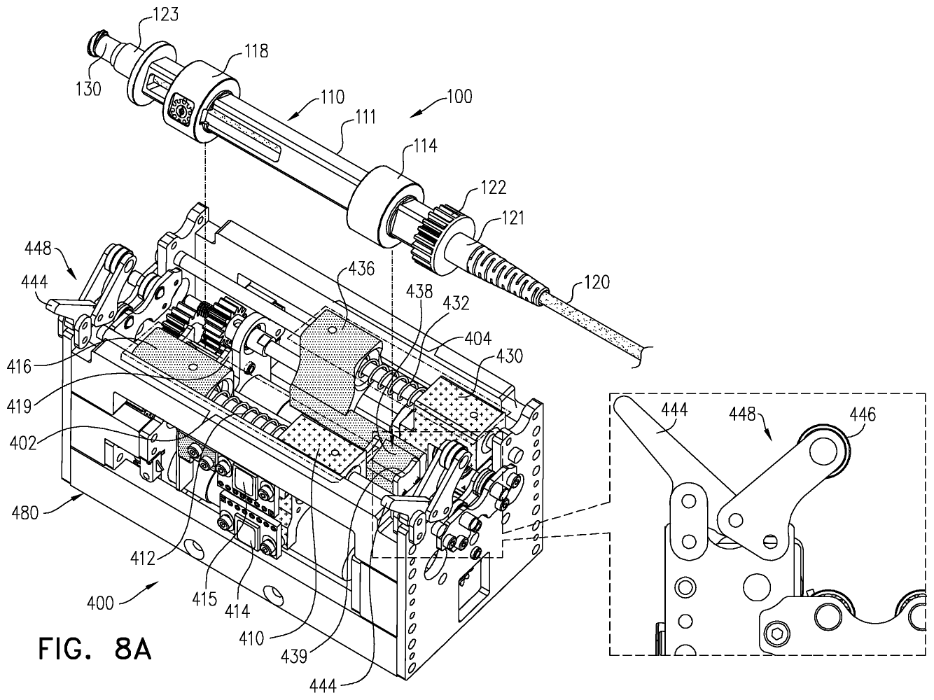

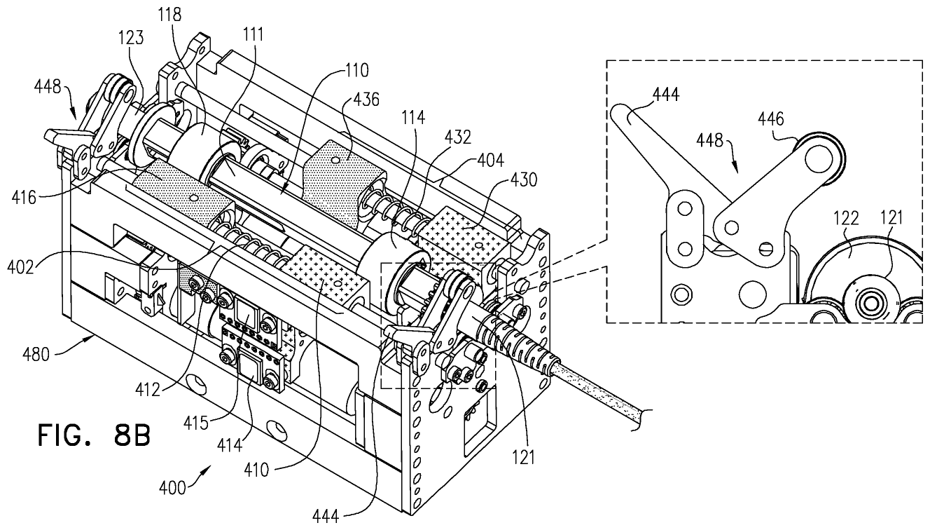

Reference is now made to FIGS. 11A-C and 12, which are schematic illustrations of robots 200a, 200b, 200c, and 200d-which are variants of robot 200, in accordance with some implementations. Each of these variants has the general characteristics of robot 200 described hereinabove—e.g. facilitating drawing of steering manipulator 400 along the advancement path by advancement manipulator 600 feeding tube 120 through the advancement manipulator. However, each of these variants comprises a manipulator structure (manipulator structures 300a, 300b, 300c, and 300d, respectively) that differs from that of robot 200—i.e. is a variant of manipulator structure 300.

As described hereinabove, advancement manipulator 600 is typically positioned as distally as possible—e.g. in proximity to the face of a subject (not shown)—in order to minimize buckling of the portion of the tube 120 that is, at any given time, distal to advancement manipulator 600. Also as described hereinabove, in order to achieve this, advancement manipulator 600 is spaced apart from steering manipulator 400. Each of variants 200a, 200b, 200c, and 200d illustrates how robot 200 may be configured to bias steering manipulator 400 to return proximally along the advancement path—e.g. in the absence of resistance by advancement manipulator 600.

Via this biasing, robot 200 may, inter alia, maintain straightness of the portion of tube 120 that is, at any given time, disposed between steering manipulator 400 and advancement manipulator 600. Maintaining this straightness of tube 120 may, inter alia, enable the position of a distal tip of steering region 140 to be more accurately determined by control system 1000.

In order to retract catheter 100 (i.e. to move the catheter proximally), advancement manipulator 600 may operate in reverse, feeding tube 120 proximally through the advancement manipulator. However, due to the flexibility of tube 120, steering manipulator 400 may not easily and/or reliably move proximally merely in response to being pushed by the proximalward feeding of the tube by advancement manipulator 600—e.g. the tube may buckle.

In order to address this, steering manipulator 400 may be biased to passively return proximally (e.g. retreat toward an initial position)—e.g. by manipulator structure 300 providing a proximal counterforce to steering manipulator 400. Due to such biasing, as advancement manipulator 600 feeds tube 120 proximally, steering manipulator 400 passively returns proximally, thereby maintaining straightness of tube 120—e.g. the part of the tube between the advancement manipulator and the steering manipulator.

Each of FIGS. 11A-C illustrates a different mechanism for biasing steering manipulator 400 to return proximally along advancement path 500. As shown in FIG. 3, advancement path 500 may be a track.

FIG. 11A shows a robot 200a, which comprises a manipulator structure 300a. In manipulator structure 300a biasing is provided by a proximal-force mechanism that comprises a counterweight 524 connected to steering manipulator 400 via a winch or a pulley that may be fixedly attached to mount 560. The pulley comprises a tether 520 extending between steering manipulator 400 and counterweight 524, and configured to hang and/or slide—e.g. around a bearing (e.g. a sheave) 522, which may be fixedly attached to mount 560. Due to gravitational pull on counterweight 524, the counterweight pulls steering manipulator 400 proximally. The steering manipulator thus pulls on tube 120 and maintains straightness of the tube. The position, mass, and shape of the weights, e.g. one for each steering manipulator, may be configured differently for different variants of the manipulator structure.

FIG. 11B shows a robot 200b, which comprises a manipulator structure 300b. In manipulator structure 300b biasing is provided by a calibrated spring 550—e.g. positioned between the proximal end of steering manipulator 400 and part of mount 560. Spring 550 may have an elasticity and spring constant calibrated to provide the desired amount of proximal force on steering manipulator 400.

FIG. 11C shows a robot 200c, which comprises a manipulator structure 300c. In manipulator structure 300c biasing is provided by the weight of steering manipulator 400 itself. For example, and as shown, advancement path (e.g. track) 500 slopes upward in a distal direction, and therefore steering manipulator is naturally biased to slide proximally down the slope. The slope of advancement path 500 may be adjusted to facilitate a desired amount of gravitational pull on steering manipulator 400.

FIG. 12 shows a robot 200d, which comprises a manipulator structure 300d. In manipulator structure 300d biasing is provided by a winch 570, which may be spring-loaded. In some implementations, and as shown, steering manipulator 400 and advancement manipulator 600 are arranged substantially perpendicular to a craniocaudal axis of a subject undergoing a procedure facilitated by the robot.

Manipulator structure 300d differs from manipulator structures 300a-c also in that its advancement path 500 is substantially vertical and/or perpendicular to the midline axis of the subject. Steering manipulators 400 of manipulator structure 300d are coupled to winches 570 via tethers 520d, and via this coupling may simply hang below the winches. The advancement path 500d of manipulator structure 300d is defined by the vector of this hanging and, in contrast to advancement path 500, may not comprise or be defined by a physical structure such as a track. Winches 570 may be configured to pull steering manipulator 400d upwards—e.g. to bias the steering manipulator to retreat proximally along the advancement path by pulling the steering manipulator upwards. Winches 570 may be spring-loaded to maintain tension on, and thereby straightness of, catheters 100.

Reference is again made to FIGS. 11A-12. The biasing (e.g. the proximal-force mechanism) of each of these manipulator structures is balanced and calibrated to maintain straightness of tube 120 (e.g. by maintaining the tube under nominal tension) between the steering manipulator and the advancement manipulator while not pulling strongly enough to cause tube 120 to stretch or to slip axially with respect to the advancement manipulator, or to cause head 110 to disconnect from the steering manipulator.

In the examples shown in 11A-12, the advancement path is substantially straight between the steering manipulator and the advancement manipulator. However, in some implementations the advancement path may be curved between the steering manipulator and the advancement manipulator.

As shown in, e.g. FIGS. 1, 2A-C, 3, and 12, advancement manipulator 600 advances tube 120 toward the subject and into a lumen, e.g. the bronchiolar airways of the subject's lung. In some implementations, in addition to advancing catheter 100, advancement manipulator 600 may be configured to rotate the catheter by rotating tube 120. For some such implementations, steering manipulator 400 does not itself actively rotate head 110, although it would typically allow the head to passively rotate—e.g. with the plungers of the head rotationally slipping within the cradles of the steering manipulator.

For some implementations, robot 200 (e.g. the manipulator assemblies 310 thereof) is configured for use with multiple variants of catheter 100. For some such implementations, robot 200 is configured for both catheters to be of the same variant as each other. For other such implementations, robot 200 may allow the use of a different catheter variant in each manipulator assembly 310—i.e. alongside each other. For example, control system 1000 may be configurable to accommodate whichever variant(s) is/are loaded into the manipulator assemblies. For some such implementations, each catheter 100 (e.g. head 110 thereof) includes an indicator indicative of the particular variant. For example, each catheter 100 may have a barcode, QR code, RFID tag, or similar, that is read by steering manipulator 400, which may forward the identity of the catheter to control system 1000, which may in turn adjust (e.g. calibrate) one or more settings accordingly—or may merely display this information to the operator.

In some such implementations in which catheter 100 has a unique identifier, control system 1000 may be configured to receive a signal that a catheter has been loaded into steering manipulator 400, triggering the control system to automatically perform a calibration step (e.g. as described with reference to FIG. 9B) and/or a stiffening step (e.g. as described with reference to FIG. 9C). In some such implementations, the catheter may be encoded with a code that is readable by the steering manipulator indicating the type of catheter, such that the control system is triggered to calibrate a specific type of catheter. Catheter tubes or steering regions may vary in numerous parameters, e.g. diameter, stiffness, and composition—e.g. depending on the width and mechanical stiffness of the lumen into which the steering region is being directed.

Reference is again made to FIGS. 1-12. As noted hereinabove, system 50 (e.g. catheters 100 thereof) may utilize a force-multiplication system in order to provide mechanical advantage to one or more of the wires within the steering region. Such a force-multiplication system may include arranging at least one of the wires of the catheter into a pulley arrangement within the steering region of the catheter. Examples of such pulley arrangements are described in Provisional U.S. Patent Application 63/247,424 to Shapira et al. filed 23 Sep. 2021, and entitled “Tubular assembly for bronchoscopic procedures,” and International Patent Application (PCT) Publication WO 2023/047219 to Shapira et al., each of which is incorporated herein by reference in its entirety. In these examples of pulley arrangements, the wire of the catheter loops back on itself at least once, such that multiple longitudinal segments of the wire run alongside each other within the steering region, thus concentrating (i.e. multiplying), within the steering region, the force applied to the proximal end of the wire at the proximal end of the catheter. Thus, such a pulley arrangement in catheter 100 would concentrate (i.e. multiply), within steering region 140, any force applied to the wire by sliding the corresponding plunger within the head of the catheter.

Such a pulley arrangement may advantageously increase the force with which the curvature of steering region 140 can be adjusted. Furthermore, such a pulley arrangement may advantageously enable the steering region to be preferentially affected by pulling on the wires—e.g. to bend preferentially compared with more proximal regions of the catheter that lack the force-multiplication arrangement of wires.

Such a pulley arrangement may also advantageously facilitate stiffening of steering region 140—e.g. due to the enhanced ability to apply force to the steering region via the wires. As further explained hereinabove with reference to, e.g. FIGS. 9C and 9G, concurrently pulling on both wires 116, 112 with balanced tensioning endows steering region 140 with a stiffness greater than it would have without the balanced tensioning. Using force-multiplication in the steering region concentrates the balanced tensioning within the steering region, thereby enabling stiffening of the steering region while minimizing axial compression or other undesired effects on (e.g. bending of) the intermediate region of the catheter proximal from the steering region.

In some implementations, the stiffenable qualities with which the pulley-based stiffening endows catheter 100 or other endoscopic tube, may be incorporated into steering region 140, and used in combination with head 110, steering manipulator 400, and robot 200, described hereinabove. In some implementations, the force-multiplication-based stiffenable steering region may advantageously be used in combination with other systems.

It may be advantageous for the intermediate region of a catheter (i.e. the region between the head and the steering region) to be highly flexible, in order to passively conform to the anatomical pathway (e.g. airway) along which it is advanced. For some implementations, it may be advantageous for the intermediate region even to be more flexible than steering region 140. As noted in U.S. 63/247,424, including force multiplication within the steering region advantageously allows a catheter to be manufactured with an intermediate region having such flexibility. (By contrast, catheters without such force multiplication often require stiffness within the intermediate region in order to allow preferential bending of the steering region.)

For some steerable catheters that utilize wires (e.g. pull-wires) for steering, artifactual effects on the curvature of the steering region of the catheter might be caused by curves formed in the intermediate region of the catheter (e.g. as it follows the anatomical pathway). Such intermediate region curves may create an imbalance between the wires of the catheter, including within the steering region, thereby artifactually bending or straightening the steering region and inadvertently/undesirably displacing the tip of the catheter. The mechanical advantage provided by the force multiplication of the pulley system is, inherently, obtained at a cost of pulling more wire to create the same amount of bending. That is, the mechanical advantage requires pulling of more wire for the same amount of change in the curvature. Thus, for catheters that include force multiplication in the steering region, this may advantageously dampen such artifactual effects on the curvature of the steering region caused by curves in the intermediate region.

In some implementations, the wires of catheter 100 may be configured with different mechanical advantages to each other. For example, one of the wires, such as straightening wire 112, may be configured with no mechanical advantage—e.g. may not be arranged in a pulley arrangement within steering region 140. For implementations in which the wires are configured with different mechanical advantage to each other (e.g. with bending wire 116 configured to have force multiplication and straightening wire 112 configured without force multiplication), robotic control system 1000 may be configured to take into account the difference in force multiplication. For example, in order to match the force applied to steering region 140 by a wire configured with mechanical advantage, a wire configured without mechanical advantage would need to be pulled harder (e.g. the force applied to its plunger would need to be greater)—e.g. twice or thrice as great.

For some implementations, in which the wires of catheter 100 are configured with different mechanical advantage to each other, head 110 and/or steering manipulator 400 may be sized to enable the different amounts of linear movement of the plungers accordingly required. For example, the slots along which the plungers slide may have different lengths, and/or the linear actuators and the cradles may be configured with different ranges of travel.

Stiffening of steering region 140 via force multiplication, e.g. using a pulley system of wires in which the force is concentrated within the steering region, may advantageously be used in combination with, e.g. vertebrae 144 described hereinabove.

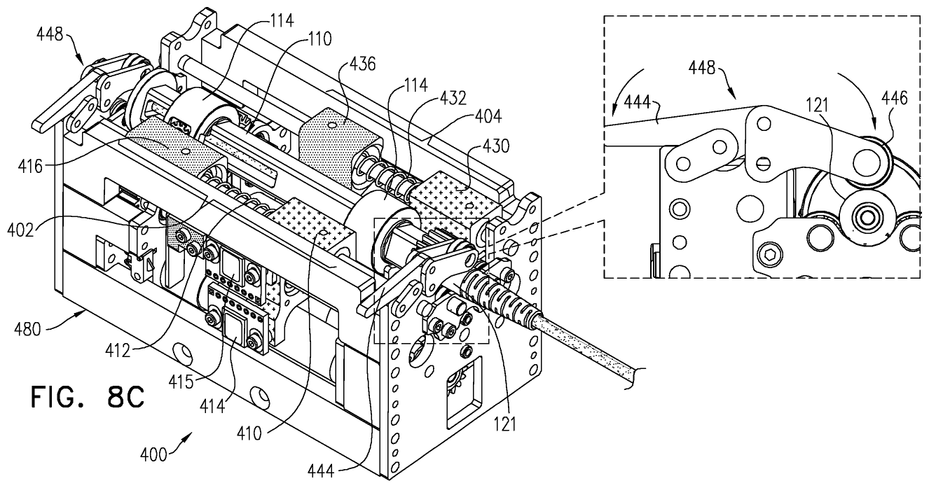

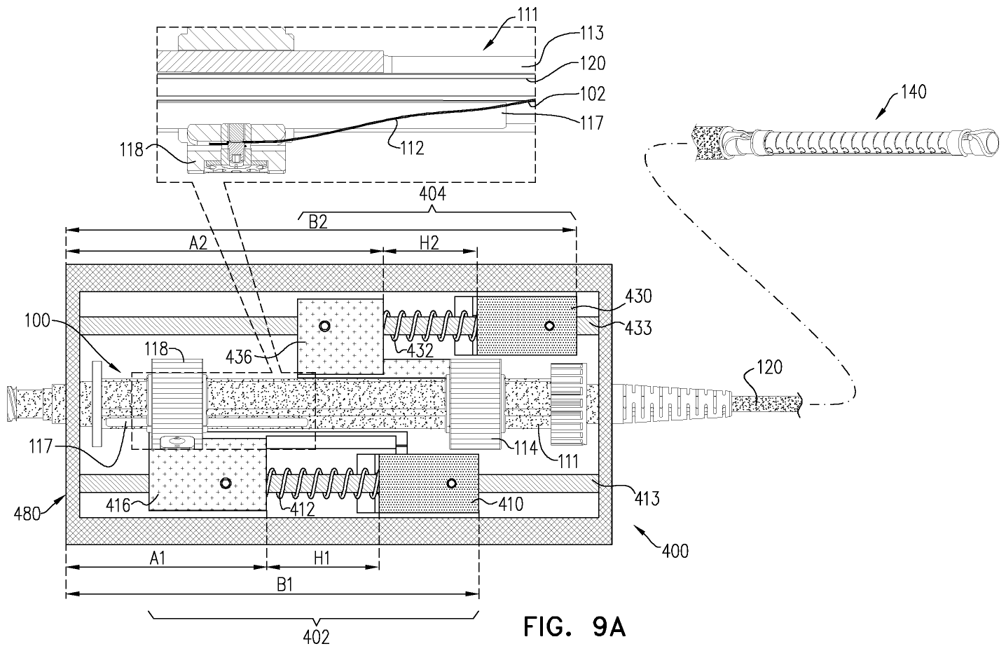

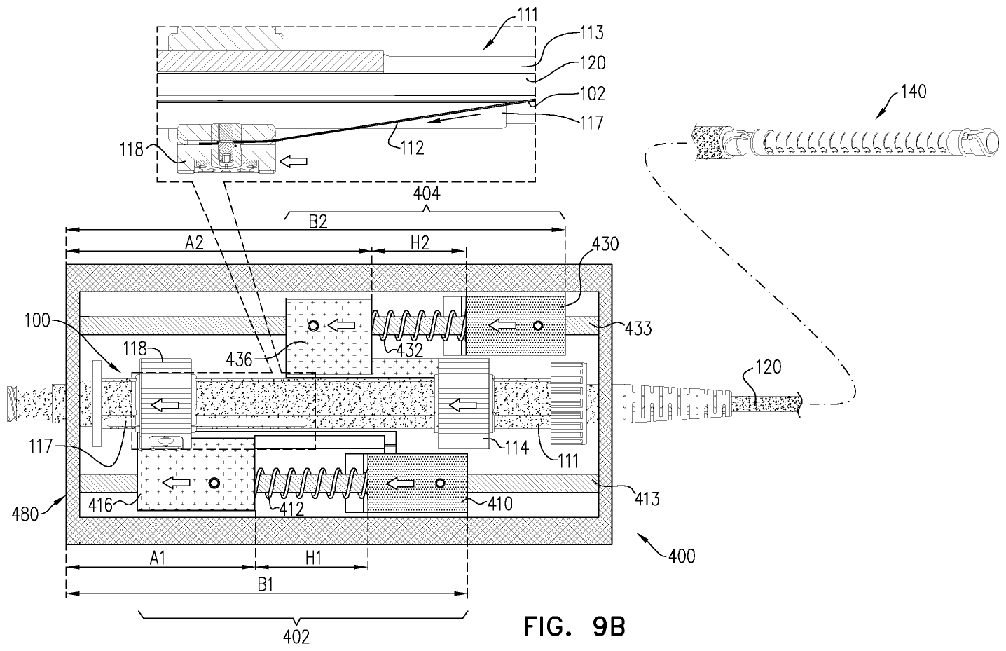

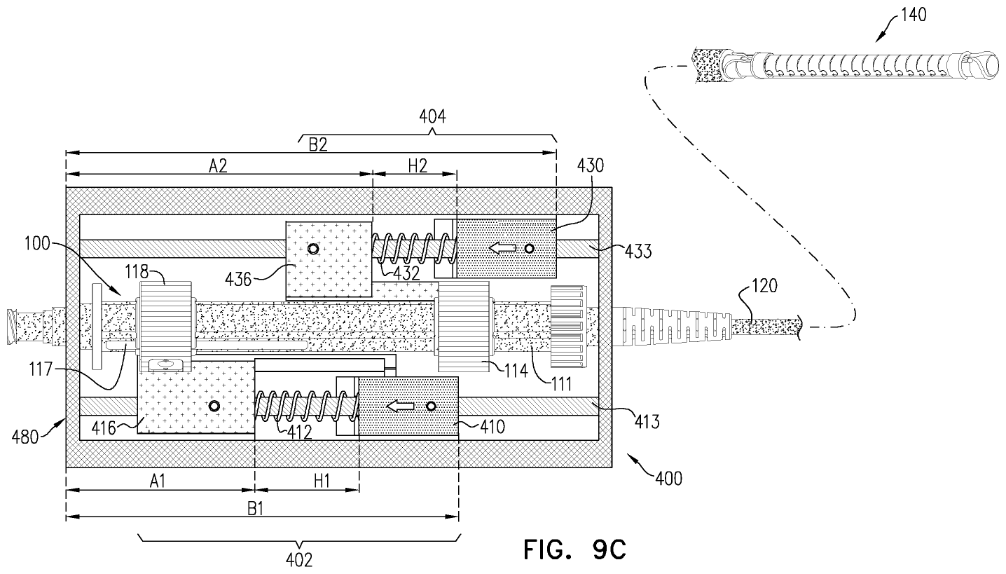

Reference is now made to FIGS. 13A-B, 14, 15, 16A-C, 17, 18A-E, 19A-B, and 20, which are diagrams and schematic illustrations that show exemplary techniques (e.g. processes, algorithms, and/or data-processing) that may be used in some implementations of the present disclosure. For some implementations, such techniques may be performed using apparatus and systems described herein—e.g. with reference to in FIGS. 1-12.

FIG. 13A schematically represents a computer-implemented method 700 (e.g. a program, or a collection of programs). In some implementations, method 700 may be carried out to pre-procedurally designate procedural target(s), catheter destination site(s), and/or catheter route(s). For example, such a catheter destination site may be an imaging site (i.e. a site to which an imaging device is to be advanced) or a tool site (i.e. a site to which a tool is to be advanced). Method 700 comprises: (a) a step 710 in which a computer model 704 of the airways of a lung of a subject is generated responsively to (e.g. is derived from) a 3D image 702 (e.g. a preoperative image set arranged into a 3D representation) of the lung; and (b) a step (e.g. an algorithm) 720 in which model 704 is utilized in building a map 706. Map 706 may include planned route(s) for one or more catheter(s), and/or may identify a target in the lung. The planning of the routes may be part of step (or program) 720.

FIG. 13B is another schematic representation of method 700, in accordance with some implementations. As shown, one or more parts (e.g. steps) of method 700 may be performed by a DPS 900. In the example shown, both step 710 and step 720 are performed by DPS 900. Some possible inputs and outputs to a DPS that performs method 700 are shown. Image 702 may comprise x-ray (e.g. CT) data, MRI data, ultrasound data, and/or data from any other imaging modus. For example, image 702 may be a 3D CT image, a 3D MRI image, and/or a set of two-dimensional images. In addition to pre-operative imaging data, other inputs may be used for generating the computer model, as further described hereinbelow. A location of a target 40 may be selected by a physician, e.g. a radiologist, reviewing image 702. Target 40 may be the lesion, tissue, or site toward which the planned bronchoscopic procedure is directed, and may comprise an input to step 710, such that a catheter route is planned to reach the target.

Model 704 may comprise a representation of the airways and may also comprise a representation of the location of target 40. The representation of the airways may be generated from image 702—e.g. by computer processing of the initial imaging data.

A map-generation step (or program) 720 generates map 706, and includes a route-generation step (or program) that generates one or more catheter routes. Map-generation step 720 utilizes model 704 as an input. For example, step 710 may feed model 704 to step 720, and/or step 720 may obtain or reference model 704 from step 710. Thus, model 704 is illustrated in FIG. 13B as an arrow from model-generation step 710 to map-generation step 720. Other inputs to map-generation step 720 may be provided. Some non-limiting examples of such inputs shown in FIG. 13B are “subject parameters” (e.g. body-mass index, smoking history, height, weight, gender, age, and past medical history including comorbidities), “hardware parameters” (e.g. accessibility of the medical tool to the target; airway geometry; size, shape, model, and/or bendability of catheter tube; size, shape, model, viewing frustum, and other details of the imaging device), and “operator preferences” (e.g. maximal allowable route length, route tortuousness, and/or route branches; user-inputted preferable angle of approach to the target; and ease of access to a candidate imaging site).

Although model generation (step 710) and map generation (step 720) are described as belonging to the same computer-implemented technique (i.e. method 700), it is to be noted that step 710 may be performed separately from step 720—e.g. at a different time (e.g. days, weeks, or months in advance of step 720), by a different DPS (e.g. on a different computer), and/or in a different location. For some implementations, step 720 is performed or facilitated by robotic control system 1000, with step 710 having been previously performed separately.

The output of model generation step 710 and/or map generation step 720 may be stored in data storage 800—e.g. local or remote (e.g. cloud-based) storage.

In the example shown, DPS 900 is discrete from robotic control system 1000 (e.g. from robot 200 as a whole)—e.g. provides an output that is (e.g. subsequently) utilized by system 1000. However, for some implementations DPS 900 is a component of robot 200. For some such implementations, DPS 900 may be a component of robotic control system 1000 (e.g. of robot 200). Irrespective of whether DPS 900 is a component of robot 200, it may be a component of system 50. Nonetheless, for some implementations, DPS 900 is separate from system 50.

For some implementations, method 700 and/or parts thereof are as described in PCT/IB2022/057505 to Shapira et al. filed Aug. 11, 2022, which is incorporated herein by reference.

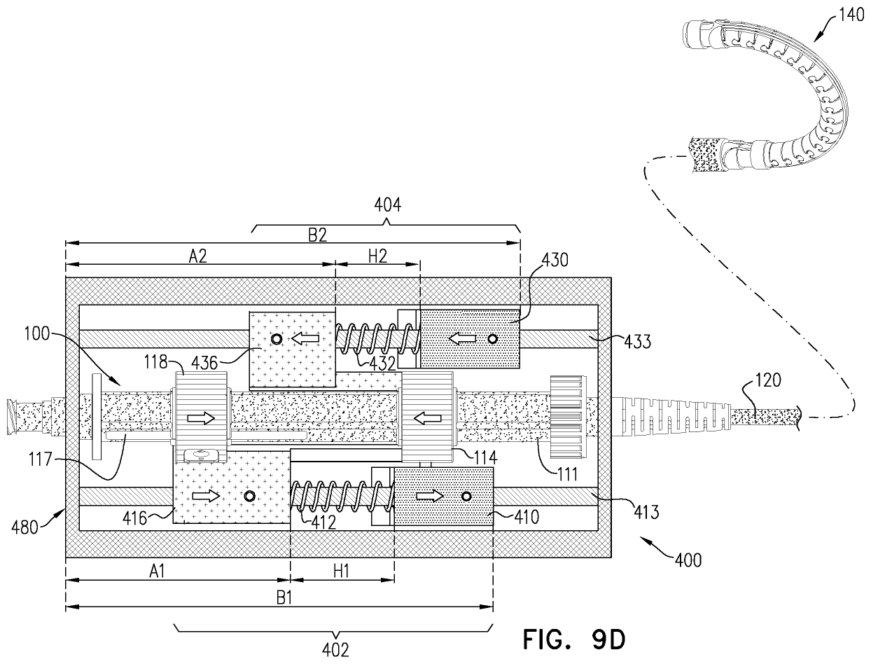

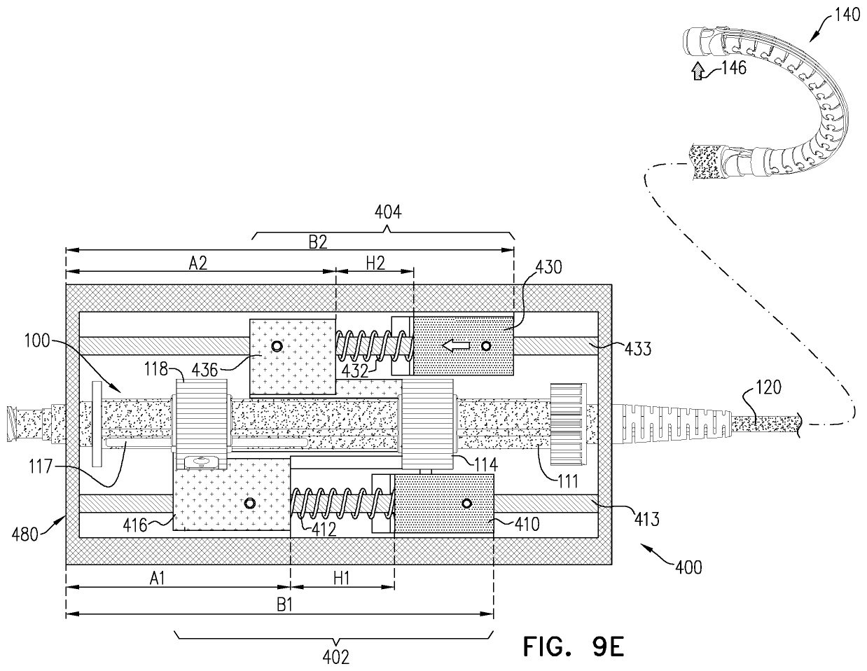

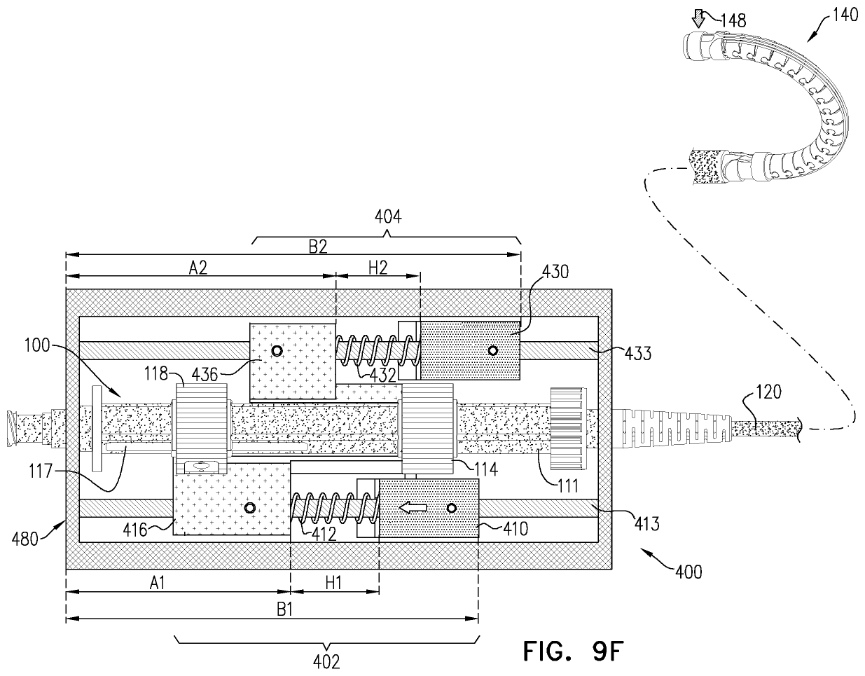

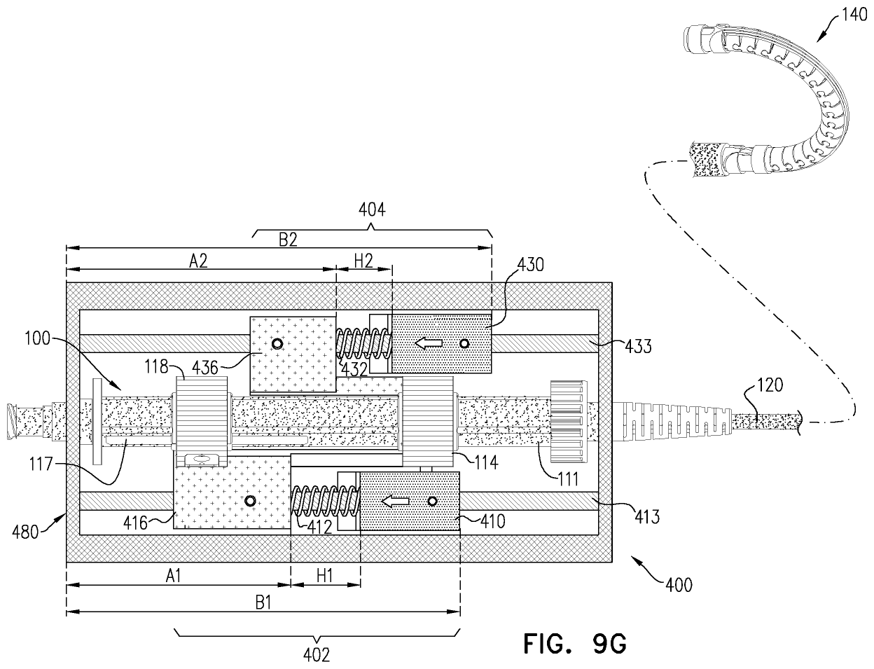

FIG. 14 is a schematic representation of an implementation in which system 50 (e.g. a variant or implementation of system 50 shown in FIG. 1) uses an output of method 700 to assist in performing a bronchoscopic procedure. This output of method 700 may include map 706 (e.g. including one or more planned routes therethrough), and may also include model 704 itself. In some implementations, and as shown, the output of method 700 is stored in data storage 800 and accessed therefrom by system 50, such as by robotic control system 1000.

In some implementations, robotic control system 1000 may comprise one or more modules for route tracing (e.g. for guiding catheter 100 along a planned route through the airways)—e.g. by assessing inputs from other components of system 50 in view of data from model 704 and/or map 706, i.e. by referencing a model of a lung of a subject, wherein the model comprises a representation of airways of the lung. For example, an advancement-tracking module 1002 may receive data indicative of linear advancement of the catheter (e.g. from sensor 610 or 620), a rotation-tracking module 1004 may receive data indicative of rotation of the catheter (e.g. from sensor 610 or 620), and/or a curvature tracking module 1006 may receive data indicative of bending of the steerable region of the catheter (e.g. from steering manipulator 400, such as in the form of a cradle output). It is to be understood these modules are described as discrete from each other primarily for the sake of clarity, and is not intended to limit these modules to a particular data or algorithm flow or structure. Moreover, these modules typically interact and/or cooperate with each other—e.g. collectively serving as a tracing module 1014. Module 1002, module 1004, and/or module 1006 (e.g. tracing module 1014) may thus track the disposition of the catheter (e.g. a distal end thereof) within the airways as an expected disposition within model 704.

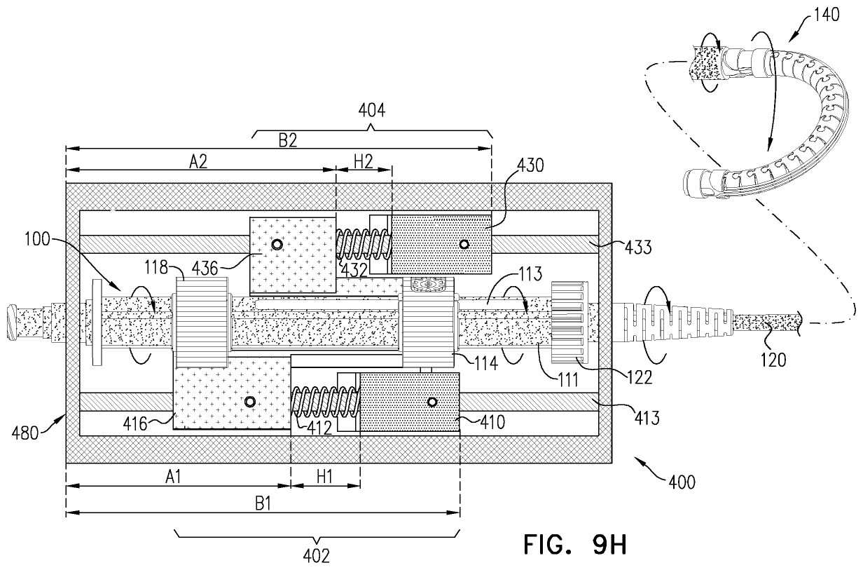

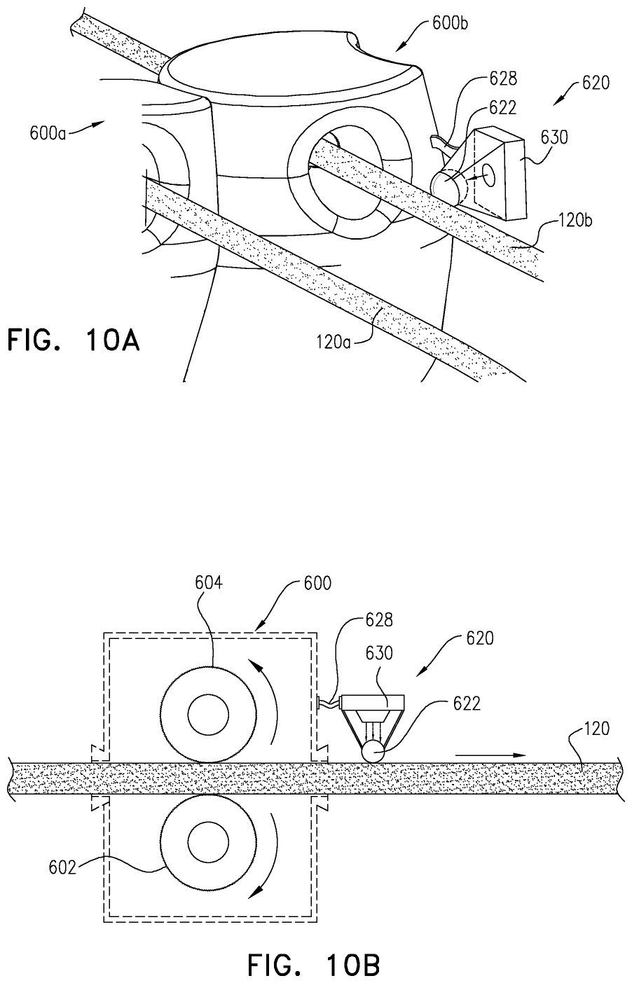

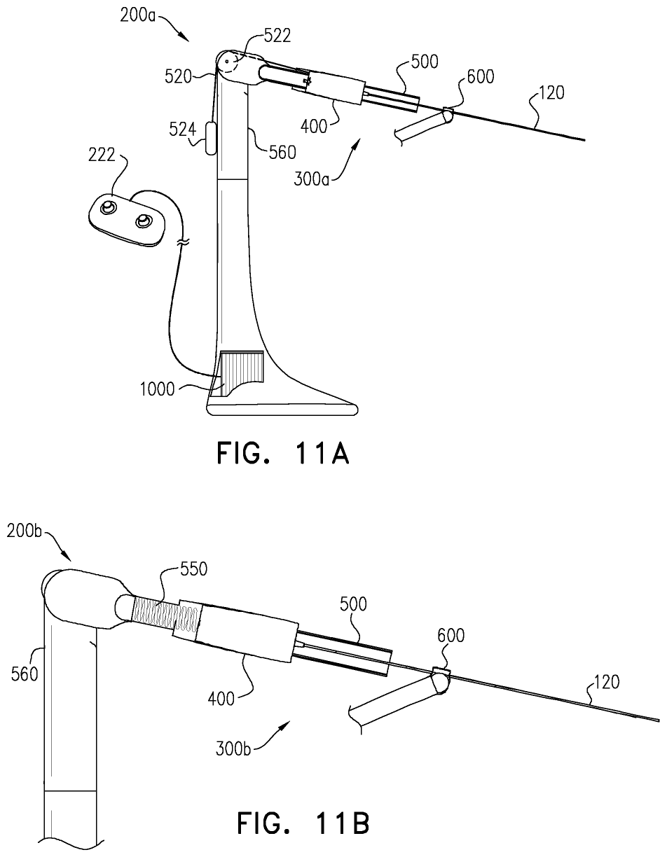

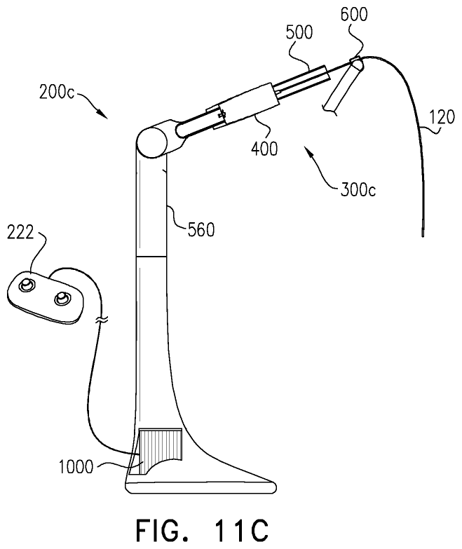

Robotic control system 1000 may include a model-image bridging module 1010 that, inter alia, processes the output of camera 132 in order to facilitate navigation of catheter 100 through the airways (e.g. by providing an image output). Model-image bridging module 1010 may be configured to generate a sequential series of viewing frustums from model 704; and/or process feedback, i.e. image input, from camera 132; and/or generate an updated model. These functions are described in further detail hereinbelow. Robotic control system 1000 may utilize a combination of these various modules and/or their functionalities and/or outputs in order to facilitate tracing of the catheter along a planned route through the airways—e.g. as described in more detail with respect to FIGS. 15-18D.

Robotic control system 1000 may include a manipulator module 1012 configured to interact with manipulator structure 300. Manipulator module 1012 may comprise, e.g. a force-tracking module 1008 that is configured to record and respond to force applied to a distal end of catheter 100, e.g. as discussed hereinabove with reference to FIGS. 7A-9H. Some further details of the force-tracking module are provided hereinbelow with respect to FIG. 16C.

As described hereinabove (e.g. with reference to FIGS. 2B-3), manipulator structure 300 can comprise at least one manipulator assembly 310, configured to engage a catheter 100 and to facilitate manipulation (e.g. advancement) of the distal portion of the catheter through the airways. This engagement of catheter 100 by manipulator structure 300 is represented in FIG. 14 by connector 350.

In some implementations, and as illustrated in FIG. 14, control system 1000 may be configured to communicate (e.g. electronically) with manipulator structure 300. This communication is represented in FIG. 14 by connector 750.

Communication 750 between control system 1000 and manipulator structure 300 may include signals (e.g. instructions) sent from the control system (e.g. from manipulator module 1012) to control the manipulator structure (e.g. a manipulator assembly 310 thereof) to manipulate catheter 100—e.g. as described with reference to FIGS. 1-9H.

Connector 350 represents the operative coupling between manipulator structure 300 and catheter 100, resulting from the catheter being loaded into the manipulator structure—e.g. as described hereinabove. For example, connector 350 may represent the ability of manipulator structure to manipulate (e.g. advance, rotate, and bend) catheter 100.

In some implementations, connectors 750 and 350 may also represent control system 1000 receiving feedback, e.g. a manipulation output, from catheter 100 and/or manipulator structure 300. For example, one or more components of manipulator structure 300 (e.g. a sensor) may provide a manipulation output indicative of sensed manipulation of the catheter. Various types of manipulation output may be routed to an appropriate module of control system 1000.

For example, and as described hereinabove (e.g. with reference to FIGS. 2C and 10A-B), manipulator structure 300 may comprise a sensor (e.g. sensor 610 or sensor 620) configured to sense advancement (e.g. linear advancement) and/or rotation of tube 120. In some implementations, the sensor is attached to and/or part of advancement manipulator 600 (e.g. as shown in FIG. 10A-B). In some implementations, the sensor may be a separate component of manipulator assembly 310. Information regarding (e.g. a signal indicative of) such advancement and/or rotation may be provided by this sensor to control system 1000, e.g. to advancement-tracking module 1002 and/or rotation-tracking module 1004, via communication 750. Thus, the sensor may provide an advancement output indicative of the sensed advancement, control system 1000 (e.g. advancement-tracking module 1002) being configured to receive this advancement output. Similarly, the sensor may provide a rotation output indicative of the sensed rotation, control system 1000 (e.g. rotation-tracking module 1004) being configured to receive this rotation output. Responsively to receiving the rotation output, control system 1000 may provide an indication of the sensor output, via communication 1250, to a user, e.g. via user interface 220, as further described hereinbelow with reference to FIG. 16C.

Further, and as described hereinabove (e.g. with reference to FIGS. 7A-9H), manipulator assembly 310 (e.g. steering manipulator 400 thereof) may be configured to output information regarding (e.g. a signal indicative of) bending of steering region 140 of catheter 100. Thus, manipulator assembly 310 may provide a curvature output indicative of the curvature of the steering region, control system 1000 (e.g. curvature-tracking module 1006) being configured to receive this curvature output.

Also as described hereinabove, steering manipulator 400 (e.g. control units 402 and 404 thereof) may be configured to provide a force output, e.g. via communication 750, indicative of a force acting on steering region 140—e.g. by sensing tension on wires 112 and 116. Changes in such tension may arise from an external force (e.g. the wall of an airway acting on steering region 140; such as described with reference to FIGS. 9E-F), or from an internal/intentional force (e.g. in order to stiffen the steering region via concurrent application of tension to wires 112 and 116; such as described with reference to FIG. 9G). Force outputs of external forces may be provided to control system 1000, e.g. to force-tracking module 1008 of manipulator module 1012. Modules 1002, 1004, and 1006 can cooperate (e.g. control system 1000 may utilize these modules) to determine an expected disposition (e.g. position and orientation) of the distal portion (e.g. the distal end) of catheter 100 within the airway, as an expected disposition within model 704.

Communication 750 may also include closed-loop functionality (e.g. feedback), such as that described with reference to FIGS. 9E-F relating to maintenance of a given configuration (e.g. curvature) of the steering region of catheter 100.

As noted hereinabove, catheter 100 may be manipulated along an airway of the lung while a camera 132 is disposed at the end of the catheter, in order to provide imaging data that can facilitate guidance of the catheter through the airways. For some implementations, system 1000 can receive an imaging input 1320 from camera 132, and can utilize the imaging input to trace the advancement of the catheter along a planned route through the airways. While camera 132 is disposed at the distal end of catheter 100, the field of view 136 of the camera may therefore be considered to be a field of view of the distal end of the catheter. Imaging input 1320 may be a video feed, or may be a series of iteratively captured images. For some implementations, system 1000 comprises a model-image bridging module 1010, which utilizes imaging input 1320 to determine and/or refine the disposition of camera 132 (and thereby the distal end of catheter 100) within model 704. For some implementations, and as further described hereinbelow, system 1000, e.g. model-image bridging module 1010, may use imaging input 1320 to refine and update model 704.

FIG. 15 is a diagram, and FIGS. 16A-E are schematic illustrations, showing a computer-implemented method by which system 50 utilizes image input 1320 to facilitate guidance (e.g. tracing) of manipulation of catheter 100 along a planned route through the airways of a lung of a subject, in accordance with some implementations. One or more parts, or all, of this guidance may be a computer-implemented method performed by system 1000—e.g. by model-image bridging module 1010 thereof.

As shown diagrammatically in FIG. 15, control system 1000 (e.g. model-image bridging module 1010 thereof), may reference (i) model 704 of the airway, and (ii) a planned route 708 through the model. In response to receiving imaging input 1320, system 1000 (e.g. image-tracking module 1010) may identify, within model 704, a viewing frustum 724 that corresponds to the field of view 136 of the imaging device—i.e. of camera 132. System 1000 (e.g. model-image bridging module 1010 thereof) may then generate an output 1300 that includes an output image 1350 derived from imaging input 1320. Superimposed on output image 1350, the system may indicate a part of planned route 708 that appears within the viewing frustum—e.g. may provide a route indication 730.

Connector 1250 (FIG. 14) represents communication between user interface 220 and control system 1000 (e.g. display module 1016). This communication may include outputs (e.g. output 1300) from control system 1000 to the user (e.g. via display 224), as well as inputs (e.g. instructions) from the user to the control system (e.g. via controller 222).

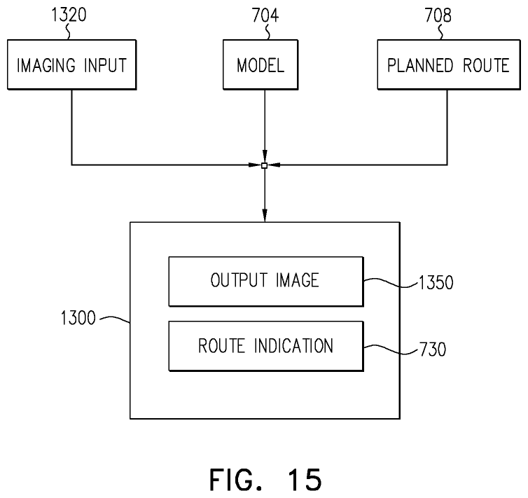

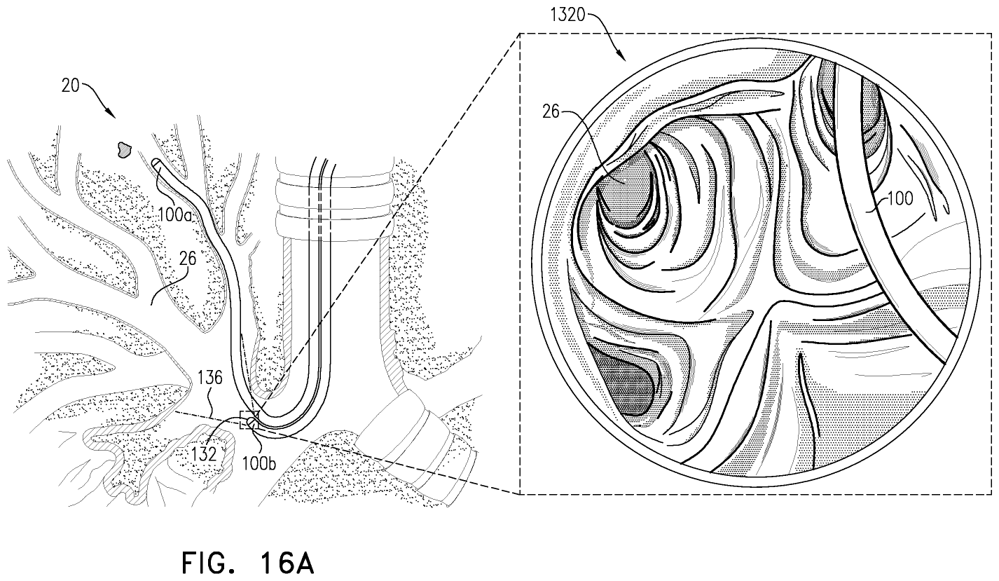

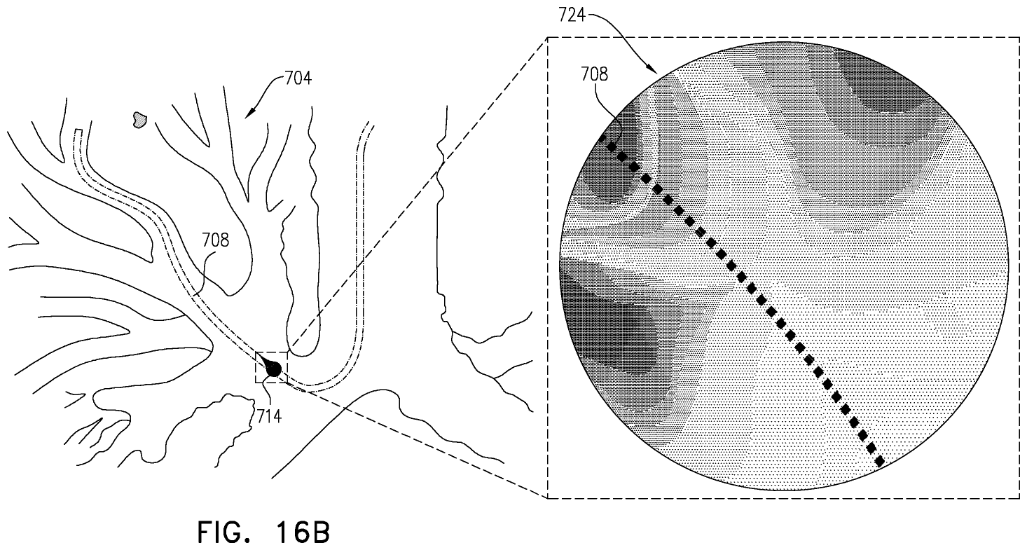

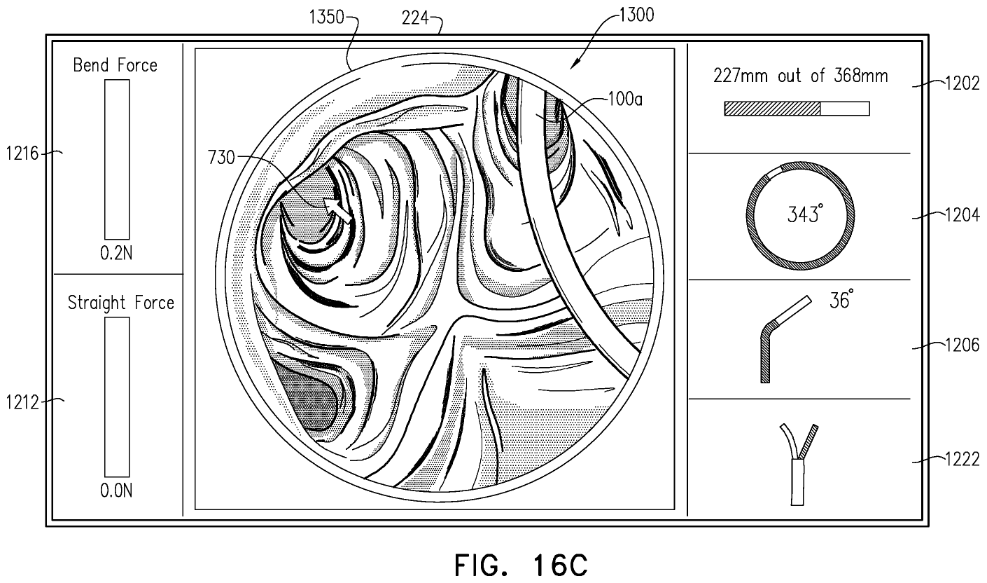

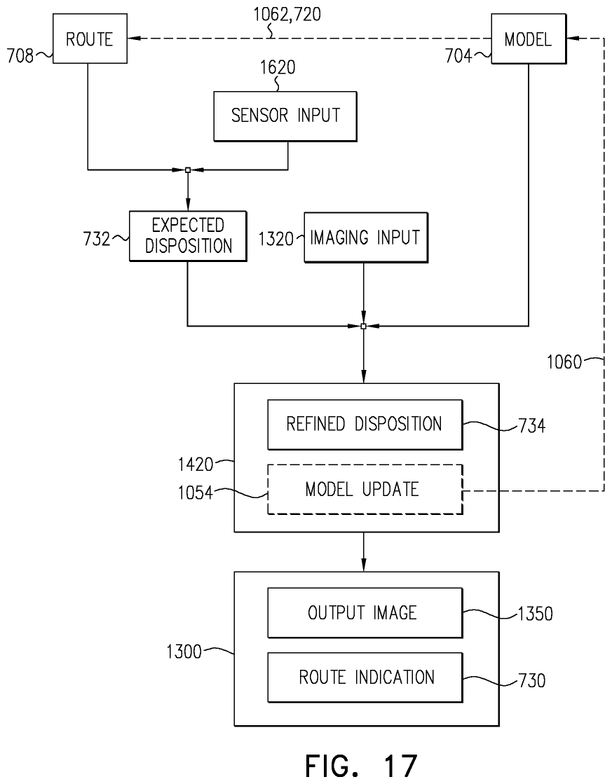

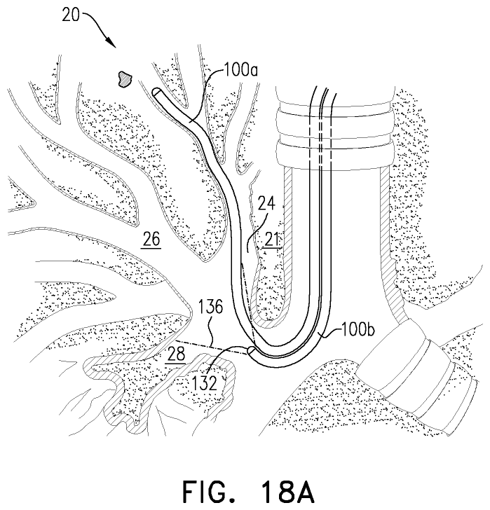

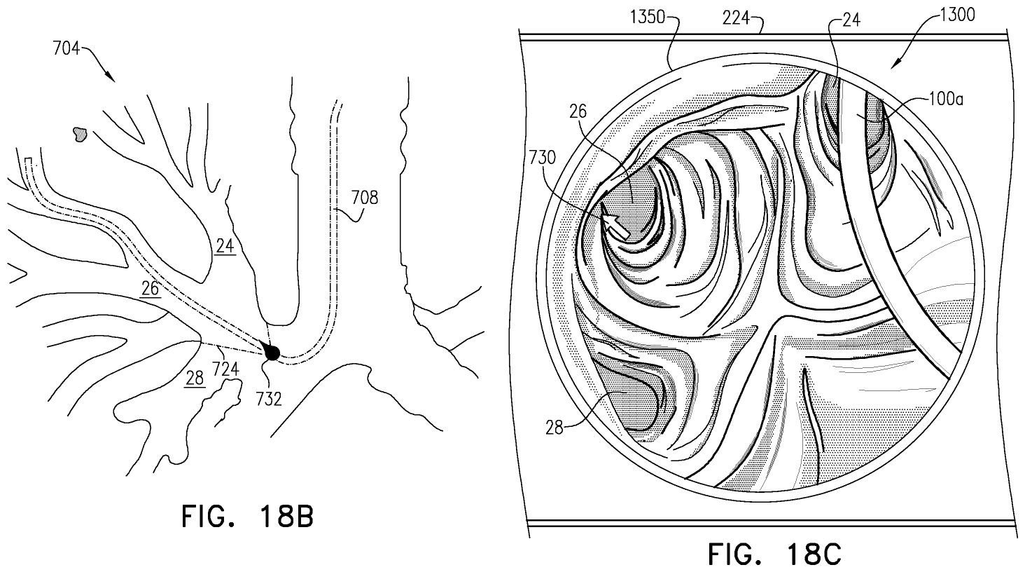

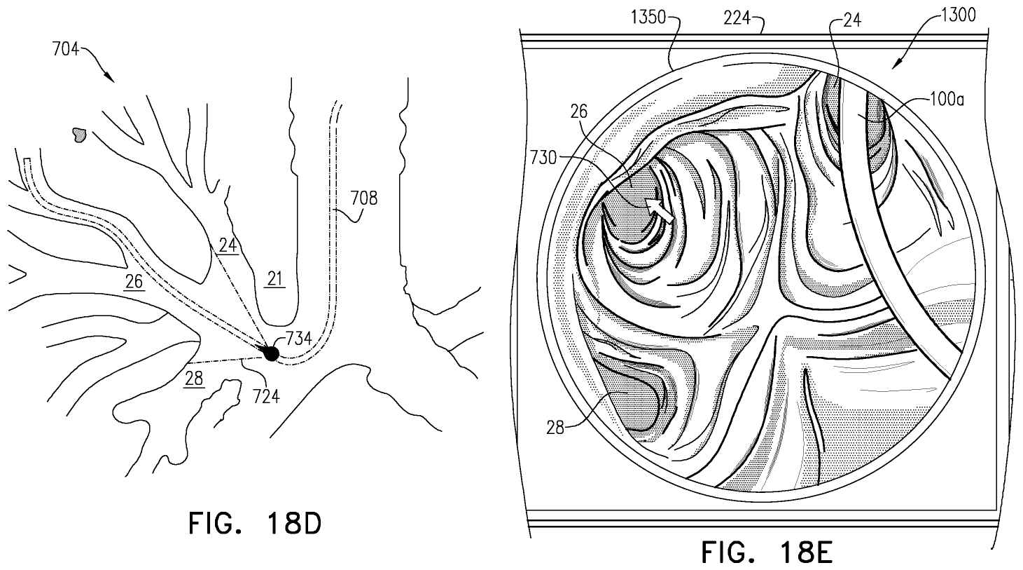

For some implementations, the method described with reference to FIG. 15 may be performed without communication from tracing module 1014—e.g. may be performed without receiving feedback from manipulator structure 300. However, for some implementations, the method described with reference to FIG. 15 may be performed responsively to such communication—e.g. as described hereinbelow with reference to FIGS. 17-18D.

FIGS. 16A-C illustrate steps in an exemplary implementation of the method shown in FIG. 15.

FIG. 16A schematically illustrates a lung 20 of a subject undergoing a bronchoscopic procedure. In some implementations, and as shown, two catheters 100, e.g. 100a and 100b, may be advanced simultaneously and/or sequentially into different airways of a lung. For the purposes of the present discussion, catheter 100a is shown as having already been advanced into, and remains disposed within, a first airway.

In FIG. 16A, camera 132 is disposed at the distal end of catheter 100b as the catheter is advanced through an airway 26. Camera 132 has a field of view 136. For some implementations, and as shown, field of view 136 faces distally from the catheter—e.g. is centered on an axis defined by the distal end of the catheter. However, it is to be understood that the scope of the present disclosure includes other fields of view.

The inset of FIG. 16A shows imaging data (e.g. an image and/or video feed) provided by camera 132. This imaging data is received by system 1000 as an image input 1320, and corresponds to field of view 136. Because catheter 100a is shown as having been already advanced through a similar part of the airways, part of catheter 100a is visible in image input 1320. In some implementations, the camera comprises a light source, such that the image input is generated while the light source illuminates the airway.

FIG. 16B represents model 704, and shows a planned route 708 for catheter 100b. As noted hereinabove, in response to receiving imaging input 1320, system 1000 (e.g. model-image bridging module 1010) may identify, within model 704, a viewing frustum 724 (inset of FIG. 16B) that corresponds to field of view 136.

Identified viewing frustum 724 has a disposition 714 (e.g. position and orientation) within model 704. Disposition 714 is schematically represented in FIG. 16B by a pointed dot. The position of the pointed dot represents the position of the viewing frustum within the model, and the orientation of the pointed dot (i.e. the direction in which it points) represents an orientation of the viewing frustum. Thus, the position of the pointed dot within model 704 (FIG. 16B) corresponds to the position of camera 132 within the actual airway (FIG. 16A), and the orientation of the pointed dot corresponds to the center of the field of view of the camera.

Inset of FIG. 16B is a schematic illustration of identified viewing frustum 724—e.g. a virtual field of view within model 704, corresponding to the field of view of camera 132. As shown, part of planned route 708 appears in viewing frustum 724. The inset of FIG. 16B may thus be considered to represent a small part of map 706 as seen from within model 704.

Viewing frustum 724 may be identified by control system 1000 (e.g. model-image bridging module 1010) comparing multiple candidate viewing frustums with imaging input 1320. For some implementations, this comparison includes, within model 704, simulating lighting conditions for each candidate viewing frustum, e.g. according to characteristics of the light source of camera 132. For example, the simulation may take into account the brightness, color, and/or diffusiveness of the light source, and/or its position and/or angle with respect to the field of view of camera 132.

For some implementations, control system 1000 (e.g. model-image bridging module 1010) performs the comparison by, inter alia, comparing brightness of pixels and/or regions within image input 1320 with those within each of the candidate viewing frustums.

FIG. 16C shows screen 224 (briefly described hereinabove, e.g. with reference to FIGS. 1-2A), on which is displayed output 1300, which includes output image 1350. As noted hereinabove, output image 1350 is derived from imaging input 1320. For some implementations, output image 1350 may be identical to imaging input 1320 (e.g. as in the example shown), or to a part thereof. For some implementations, control system 1000 performs one or more image-processing techniques on imaging input 1320, such that output image 1350 differs, possibly significantly, from the more “raw” imaging input.

Superimposed on output image 1350 is an indication 730 of a part of planned route 708. This part of the planned route may be an upcoming part—e.g. the part of the route immediately beyond (i.e. distal to) camera 132, and thereby the distal end of catheter 100. Screen 224 may further display additional information, e.g. regarding a state of the distal end of catheter 100b, and/or forces being experienced by the distal end of the catheter, as further described hereinbelow.

As shown, indication 730 may be a simple indication such as an arrow that indicates a direction and/or branch that should be taken in order to continue along the planned route. In some cases, indication 730 may comprise a series of dots, a distinct marking/coloration of the airway (e.g. the ostium of the airway) comprising the segment, or other manner of distinguishing the planned route. Furthermore, for some implementations, control system 1000 may alert the operator should an incorrect branch be taken—e.g. using indication 730 and/or another visual and/or audible alert.

The superimposition of indication 730 of an upcoming part of the planned route on output image 1350 advantageously provides the user with information that may materially improve the speed and accuracy of the procedure.

FIG. 17 is a diagram, and FIGS. 18A-E are schematic illustrations, showing system 50 utilizing techniques described with reference to FIGS. 15-16C to refine an expected disposition of the catheter tip, in accordance with some implementations. Thus, for some implementations, the ideas described with reference to FIGS. 17-18E may be considered to be implementations of those described with reference to FIGS. 15-16C. These computer-implemented methods may be performed by control system 1000, and/or by a separate DPS.

At any point during the procedure (e.g. repeatedly throughout the procedure), an expected disposition 732 of the distal tip of catheter 100 may be determined—e.g. by control system 1000 (e.g. by tracing module 1014 and/or components thereof, such as distance-tracking module 1002, rotation-tracking module 1004, and/or curvature-tracking module 1006).

Determination of expected disposition 732 may be made responsively to trusted information regarding the advancement, rotation, and/or bending of the catheter. For example, if catheter 100 has been advanced a known distance since the start of the procedure (e.g. tracked by distance-tracking module 1002), its expected disposition may be, or at least include, that distance along the planned route. Similarly, trusted degrees of rotation (e.g. tracked by rotation-tracking module 1004) and/or curvature of the catheter (e.g. tracked by curvature-tracking module 1006) may contribute to the expected disposition—e.g. a direction in which the tip of the catheter is facing.

For some implementations, such trusted information may be a record and/or cumulation of instructions (e.g. a quantitative sum thereof) sent by control unit 1000, to manipulator structure 300 (e.g. to manipulator assembly 310), to manipulate catheter 100.

For some implementations, such trusted information may be provided as one or more inputs (e.g. feedback) from manipulator structure 300 (e.g. part of communication 750; FIG. 14). For some such implementations, and as shown, outputs from manipulator structure 300 may serve as sensor inputs 1620. For example, the cradle output from one or both cradle encoders of steering manipulator 400 (which, as described hereinabove, is indicative of the curvature of steering region 140 of catheter 100) may be received (e.g. monitored) by curvature-tracking module 1006, an output from sensor 610 or 620 that is indicative of advancement of tube 120 of catheter 100 may be received (e.g. monitored) by distance-tracking module 1002, and/or an output from sensor 610 or 620 that is indicative of rotation of the tube of the catheter may be received (e.g. monitored) by rotation-tracking module 1002. Thus, expected disposition 732 may be determined responsively to sensor input(s) 1620 and planned route 708.

Control unit 1000 (e.g. display module 1016 thereof) may be configured to provide one or more navigational guides (e.g. indications) responsively to expected disposition 732. Display module 1016 may communicate expected disposition 732 via communication 1250 to user interface 220, e.g. to be displayed on screen 224. For some implementations, indication 730 is provided responsively to expected disposition 732. For example, the viewing frustum, in which appears the part of the planned route that indication 730 indicates, may be identified responsively to the expected disposition. Indication 730 may then be superimposed on output image 1350, e.g. as diagrammed in FIG. 15 and as described with reference to FIG. 16C, mutatis mutandis.

For some implementations, and/or in some instances, despite expected disposition 732 having been determined responsively to trusted information such as sensor input 1620, the expected disposition within model 704 (e.g. along planned route 708) may nonetheless not coincide precisely with the true disposition of the catheter tip within the airways. This may occur, for example, due to limitations in the accuracy of 3D image 702 and/or generation of model 704, progressive changes in the anatomy between acquisition of 3D image 702 and performance of the procedure, differences in the pose and/or positioning of the subject between acquisition of 3D image and performance of the procedure, and/or dynamic fluctuations in the anatomy due to movement and/or breathing of the subject.

Therefore, in order to improve the accuracy of the procedure, in some implementations, the control system may be configured to refine expected disposition 732 and/or model 704 by following a series of steps, e.g. as shown in FIGS. 17-19B. In the example shown, this refinement is used to facilitate advancement of catheter 100b, but it is to be understood that the techniques described are similarly applicable to catheter 100a, or to a single-catheter system.

When disposed at the distal tip of catheter 100b, camera 132 is therefore disposed at the true position of the tip of the catheter within the airways. Thus, the field of view 136 of camera 132 may be representative and indicative of a true disposition of the tip of catheter 100b within the airways of lung 20 (FIG. 18A).

FIG. 18B illustrates model 704, showing a planned route 708 for catheter 100b. However, unlike that shown in FIG. 16B, the model shown in FIG. 18B has some imprecision relative to the actual lung 20 that it represents: The region of the lung that includes airways 24, 26, and 28 is skewed in model 704 compared to the lung shown in FIG. 18A—e.g. observe that spacing 21 between airway 24 and the trachea is greater in FIG. 18B than in FIG. 18A. Thus, although field of view 136 of camera 132 (FIG. 18A) includes (i) an ostium into airway 24 (through which catheter 100a is disposed), (ii) an ostium into an airway 26, and (iii) an ostium into an airway 28, viewing frustum 724 of expected disposition 732 (FIG. 18B) does not include the ostium into airway 28—e.g. due to the above described imprecision of model 704. In some implementations, such imprecision may be reflected in an inaccuracy of the route indication 730 when superimposed on the output image 1350, e.g. as shown in FIG. 18C (e.g. as compared with FIG. 16C).

Subsequent to determining expected disposition 732, and in order to increase the accuracy of indication 730, expected disposition 732 (e.g. derived from sensor inputs, as described hereinabove) may be refined by control system 1000—e.g. into a refined disposition 734 (FIG. 18D). A refinement process 1420 (FIG. 17) is performed by referencing imaging input 1320 with respect to model 704 and expected disposition 732, and may be achieved by utilizing techniques described with reference to FIGS. 15-16C, mutatis mutandis. For example, control system 1000 (e.g. model-image bridging module 1010) may compare multiple candidate viewing frustums with imaging input 1320, in order to identify viewing frustum 724, which is the viewing frustum that suitably matches the field of view of the imaging input from camera 132. For some implementations, control system 1000 may be configured to begin searching in a region of model 704 that is close to (e.g. within a threshold distance of) expected disposition 732, and to progressively widen the search until viewing frustum 724 is identified.

In the assessment of a given candidate viewing frustum, its proximity to expected disposition 732 may be taken into account by control system 1000. For some implementations, module 1010 may give significant weight to this proximity, e.g. such that, in some cases, the candidate viewing frustum that is identified as viewing frustum 724 may not be the candidate viewing frustum that, from an imaging perspective, most closely resembles imaging input 1320. Rather, its sufficiently close resemblance combined with its closer proximity to expected disposition 732 may result in it being identified as viewing frustum 724. It is to be noted that, in this context, “proximity” may refer to translational distance and/or to rotational distance. For example, in a given case, a first candidate viewing frustum may be assigned greater weight than a second candidate viewing frustum if the orientation of the first candidate viewing frustum is sufficiently closer to that of the expected disposition-even if the first candidate viewing frustum is translationally further from the expected disposition. Similarly, the inverse may apply in other cases.

As mentioned hereinabove, camera 132 may use a light source to acquire images during a bronchoscopy procedure. Thus, the identification of viewing frustum 724 may be facilitated by simulating lighting conditions in model 704 corresponding to lighting conditions provided by an actual light source used by camera 132. Simulating the lighting conditions may comprise, e.g. calculating reflections of light from a virtual light source within the model—e.g. reflections off of the walls of the airways.

Depending on the requirements of a particular procedure and/or human operator and/or imaging device, control system 1000, e.g. model-image bridging module 1010, may be programmed to assign greater weight to a specific factor, e.g. translational proximity to an expected disposition, rotational proximity to an expected disposition, similar lighting conditions, relative diameters and positions of ostia visible in a candidate viewing frustum, and/or other factor(s).

By first referencing expected disposition 732 of the catheter tip, control system 1000 (e.g. model-image bridging module 1010) may reduce the number of candidate viewing frustums needed to compare with image input 1320 in order to identify viewing frustum 724. Thus, the utilization of expected disposition 732 may significantly increase the efficiency of identifying viewing frustum 724 (and thus the feasibility of doing so intra-procedurally, e.g. in real-time)—e.g. compared to referencing the entirety of model 704 (e.g. every candidate viewing frustum within the model).

FIG. 18D illustrates the refined disposition 734 that has been determined by control system 1000. Viewing frustum 724 of refined disposition 734 includes the ostium of (i) airway 24, (ii) airway 26, and (iii) a portion of the ostium of airway 28. Refined disposition 734 (FIG. 18D) is thus more representative than expected disposition 732 (FIG. 18B) of the true disposition of the catheter tip and camera 132 (FIG. 18A).

Due to refinement process 1420, output 1300 may be improved. FIG. 18C shows an output 1300 based on expected disposition 732—note that indication 730, which should indicate the ostium of airway 26, is slightly off-target. In contrast, the output 1300 shown in FIG. 18D, which is based on refined disposition 734, advantageously points more directly at the ostium of airway 26, thereby providing clearer guidance to the operating physician.

In some implementations, as part of refinement process 1420, system 1000 may update model 704 to more closely represent airways of lung 20—i.e. may perform a model update 1054 (FIG. 17). The updated model 704 may be stored in data storage 800. Arrow 1060 represents this feedback by which model 704 is updated. Model update 1054 may be performed by model-image bridging model 1010, and/or by a model update module of system 1000. An example of such an update is illustrated in FIGS. 19A-B. Module update 1054 may be performed alternatively or in addition to the disposition refinement described with reference to FIGS. 18A-E.

FIG. 19A is a schematic illustration of model 704, showing planned route 708 for catheter 100b, similar to the view shown in FIG. 18B, in which viewing frustum 724 does not include the ostium into airway 28—e.g. due to imprecision of model 704.

FIG. 19B is a schematic illustration of model 704 after model update 1054 has been performed. In the example shown, the imprecision of model 704 includes inaccurate skewing of certain airways (e.g. the orientation of part of the model with respect to another part of the model) relative to their arrangement in the actual lung. FIG. 19B represents this imprecision being corrected such that model 704 more closely represents lung 20 of the subject—e.g. observe that the spacing 21 on model 704 in FIG. 19B is more representative of that in lung 20 (FIG. 18A) than is spacing 21 in FIG. 19A. This de-skewing is indicated by arrows pointing in a generally clockwise direction, indicating that this part of model 704 has been rotated and/or deflected toward the trachea. As a result, in the updated model (FIG. 19B), viewing frustum 724 includes the ostia to (i) airway 24, (ii) airway 26, and (iii) part of the ostium to airway 28—i.e. more closely resembling field of view 136 (FIG. 18A). The difference in viewing frustum 724 between FIGS. 19A and 19B is similar to the difference in viewing frustum 724 between FIGS. 18C and 18E. In both examples, greater resemblance of the airways, the model, and the planned route has been achieved by updating model 704 and/or planned route 708, based on sensor input and/or imaging input.

Although FIGS. 19A-B illustrate updating the model via de-skewing, other forms of updating may be required and performed. For example, model update 1054 may comprise scaling the model (or part thereof) to correct a difference in scale between the model (or part thereof) and the actual lung (or part thereof). In some implementations, model update 1054 may comprise stretching the model in one or more dimensions to reduce such differences along one or more axes.

In some implementations, model update 1054 may be performed subsequently to a comparison of imaging inputs obtained at sequential positions of the camera along the airway, e.g. along the planned route. After matching each field of view of the camera with a corresponding viewing frustum, model update 1054 may comprise adjusting a part of the model corresponding to a segment between the two viewing frustums, to match more closely the imaged anatomy of the airway. In some cases, model update 1054 may merely comprise adjusting a distance between consecutive forks, i.e. choice points, of the planned path along continuing bifurcations of the airway, and/or an angle between those forks.

In some implementations, dramatic changes to the anatomy may occur between the initial imaging studies 702 and the procedure itself, e.g. due to growth of a tumor that may block an ostium along the initially planned route. In cases in which such a significant discrepancy is found between model 704 and imaging input 1320, updates to model 704 and/or changes to planned route 708 may be performed. If a threshold difference between an airway and model 704 has been exceeded, system 1000 and/or another DPS may automatically plot and suggest an alternative route.

It is to be noted that FIGS. 18A-E and 19A-B illustrate corrective actions taken by control system 1000, or another DPS, to provide real-time updates to model 704, and/or planned route 708, to more closely correspond to lung 20 of the subject as observed during the procedure. Updating the model may advantageously enhance the accuracy of subsequent steps of the procedure.

Repeating model update 1054 throughout the procedure (e.g. in real-time or near real-time) may advantageously cumulatively decrease discrepancies between model 704 and the lung. Thus, as the catheter moves along the planned route, successive expected dispositions 732 will, progressively, more closely match the true disposition of camera 132, and thereby the tip of catheter 100. Therefore, in successive iterations of refinement process 1420, by combining model update 1054 with disposition refinement, the degree to which expected disposition 732 differs from field of view 136 of camera 132 within the airway may diminish as the procedure advances toward the target.

In contrast, performing disposition refinement alone, i.e. without model updates, may require repeated significant and/or increasing refinements of the expected disposition 732 relative to the true disposition of camera 132 at the catheter tip within the airway.

In some implementations, the updating of model 704 may result in system 50 (e.g. control system 1000) or DPS 900 altering (or selecting an alternative) planned route 708—e.g. a portion of the planned route beyond the current disposition of the catheter tip. For example, in light of the updating of model 704, system 50 or DPS 900 may determine that the current planned route is, in fact, not the most optimal. Arrow 1062 represents this feedback by which route 708 (e.g. map 706 to which the route belongs) is updated. For some implementations, map-generation step (or program) 720 may perform this route updating step based on updated model 704—e.g. similarly to how it generated the original map and route(s), as described with reference to FIGS. 13A-B.

It is to be noted that for multi-route (e.g. multi-catheter) procedures, the updating of model 704 during the advancement of one catheter may alternatively or additionally result in the updating of another route (e.g. for a catheter that is yet to be advanced).

In some implementations, a physician user or operator may intra-procedurally choose to select a more suitable planned route responsively to refinement process 1420, e.g. based on updates to model 704. In such cases, the user may manually update planned route 708 via communication 1250 to control system 1000 or to another DPS.

Reference is again made to FIGS. 14 and 16C. In some implementations, and as shown, robotic control system 1000 (e.g. display module 1016) may be configured to communicate with user interface 220, e.g. to send signals to user interface 220, as represented by communication 1250 in FIG. 14. Communication 1250 represents two-way communication between control system 1000 and user interface 220, such that a physician or other user may operate robotic control system 1000 via communication 1250. Human control of the robotic control system 1000 may be accomplished by, e.g. issuing commands, and/or by using a hand-held controller 222, e.g. as shown in FIGS. 1, 2A-B, 11A, to control manipulator structure 300 via robotic control system 1000. Indications of signals and feedback from control system 1000 to user interface 220 may be shown, e.g. on screen 224 (FIG. 16C).

In some implementations, control system 1000, e.g. force-tracking module 1008 of manipulator module 1012, is configured to receive force outputs from steering manipulator 400 (e.g. as described with reference to FIGS. 9A-H). Force outputs (or derivatives thereof) may be displayed on screen 224. In FIG. 16C, a force output 1212 represents tension in straightening wire 112, and force output 1216 represents tension in bending wire 116. Control system 1000, e.g. force-tracking module 1008, may regulate force outputs 1212, 1216 in order to, e.g. prevent the tension magnitude in the corresponding wire from rising above a predetermined level. In some implementations, control system 1000 may warn an operator about the tension magnitude in the corresponding wire rising above a predetermined acceptable limit.

In some implementations, control system 1000, e.g. advancement-tracking module 1002 and/or rotation-tracking module 1004, may be configured to receive outputs from a sensor, e.g. sensor 610 or 620, indicative of advancement (e.g. an advancement output 1202) and/or rotation (e.g. a rotation output 1204) of catheter 100, respectively, as discussed hereinabove. Curvature-tracking module 1006 may be configured to receive, from a sensor, such as of control units 402 and/or 404 of steering manipulator 400, outputs indicative of curvature of steering region 140 of catheter 100 (e.g. a bending output 1206), e.g. as described with respect to FIGS. 9D-F. Specific sensor outputs may be depicted, e.g. by a qualitative and/or quantitative visual display, on screen 224. Such displayed sensor outputs may include advancement output 1202, rotation output 1204, and/or bending output 1206. These outputs may also be monitored by control system 1000 to notify the user, e.g. of parameters outside of a pre-determined reference range.

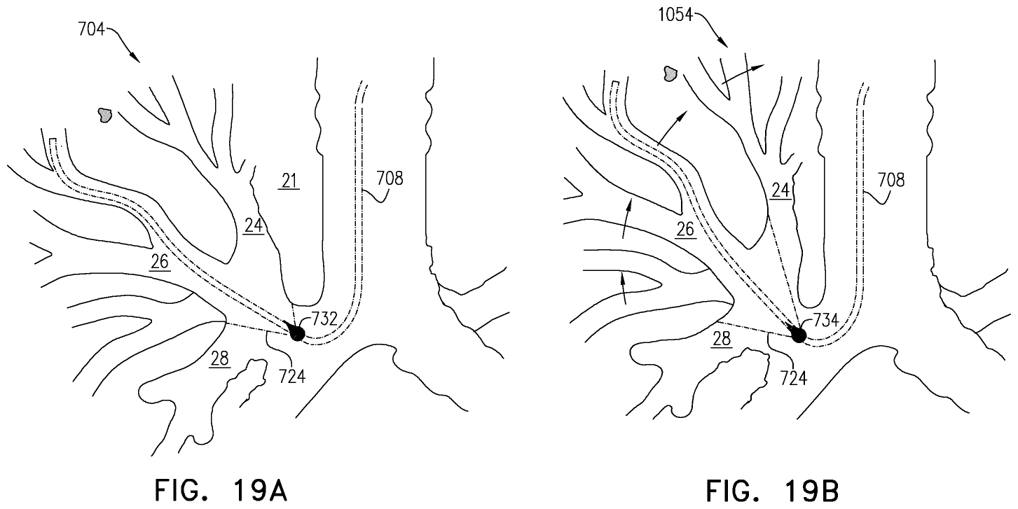

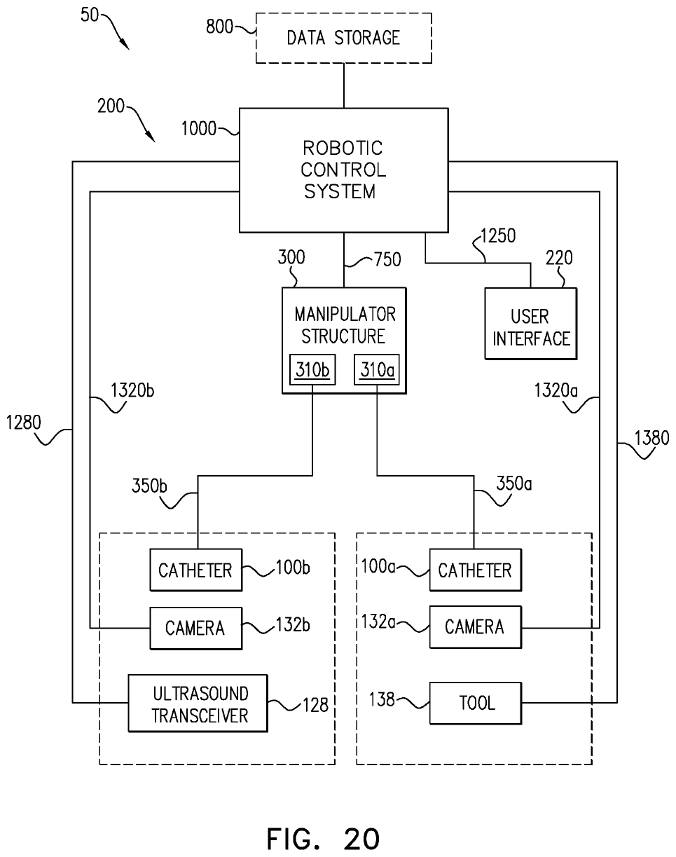

Reference is now made to FIG. 20. In some implementations, and as described hereinabove (e.g. with reference to FIGS. 1-3), system 50, i.e. manipulator structure 300 of robot 200, may include more than one (e.g. two) manipulator assemblies 310 (310a, 310b), each manipulating a corresponding catheter 100 (100a, 100b). FIG. 20 is a schematic illustration that represents such an implementation. In FIG. 20, connectors 350a and 350b represent the operative coupling between each manipulator assembly 310 and its corresponding catheter 100.

A first catheter 100, e.g. catheter 100a, may carry, simultaneously or sequentially, a camera 132a and a tool 138 for carrying out a procedure on the lung 20 of the subject. A second catheter 100, e.g. catheter 100b, may carry, simultaneously or sequentially, a camera 132b and an ultrasound transceiver 128, which is used to observe, position, and monitor (e.g. facilitate control of) the use of tool 138.

For some implementations, tool 138 and/or ultrasound transceiver 128 is advanced through its respective catheter only once the distal end of the catheter has successfully reached the respective site (e.g. the tool site or the imaging site). For some such implementations, the respective camera is withdrawn from the catheter to facilitate this advancement. This may be as described in International Patent Application PCT/IB2022/057505 to Shapira et al. filed Aug. 11, 2022, which is incorporated herein by reference.

Thus, robotic control system 1000 may be configured to manipulate each (e.g. both) catheters 100—e.g. to facilitate an operator advancing both tubes 120a and 120b towards their respective sites. The operator or user may interact with system 50 via user interface 220. In a two-catheter system, control system 1000, e.g. display module 1016, may allow the operator to switch control of manipulation of one catheter (e.g. one manipulator assembly 310) to that of the other catheter, and may further provide, via user interface 220 (e.g. screen 224 thereof) an indication of which of the two catheters is presently under manipulation e.g. as shown as output 1222 (FIG. 16C). The displays on screen 224 may thus be configured to show outputs related to the specific catheter currently under manipulation. Similarly, display module 1016 may further provide an indication of a disposition (e.g. in three dimensions) of the distal end of each catheter, as determined by the tracing module. Display module 1016 may further provide an indication of forces (1212, 1216) acting on the distal tip of each catheter.

The intended destination of the ultrasound transceiver 128 and the tool 138 (i.e. an imaging site and a tool site, respectively) are typically present in (e.g. pre-entered into) map 706, such that the route-tracking/tracing module can assess whether the distal end of each catheter is correctly positioned at its respective site at the endpoint of the route. Tracing module 1014 typically operates in tandem with manipulator module 1012, e.g. data regarding catheter position is shared between and used by both modules, and displayed to the user via display module 1016.

In some implementations, control system 1000 is configured to manipulate tool 138. This is indicated in FIG. 20 as communication 1380, which may represent an electrical and/or mechanical connection. Communication 1380 may be discrete from manipulator structure 300 (e.g. as shown), or may be via the manipulator structure. Likewise, feedback from tool 138 may be provided to control system 1000 via communication 1380. Alternatively, tool 138 may be manipulated independently of control system 1000. For example, tool 138 may be a mechanical tool that is controlled by a physician manually or robotically.

Examples of tool 138 include a biopsy needle, a blade, a suction device, scissors, jaws, an energy applicator such as for electrocautery or ablation—e.g. a radiofrequency (RF) energy applicator, a high-intensity focused ultrasound (HIFU) applicator, and/or a substance applicator (e.g. for applying a drug, or a caustic substance).

In some implementations, control system 1000 is configured to manipulate ultrasound transceiver 128. This is indicated in FIG. 20 as communication 1280, which may represent an electrical and/or mechanical connection. Communication 1280 may be discrete from manipulator structure 300 (e.g. as shown), or may be via the manipulator structure. Likewise, feedback from ultrasound transceiver 128 may be provided to control system 1000 via communication 1280. Alternatively, ultrasound transceiver 128 may be manipulated independently of control system 1000. For example, ultrasound transceiver 128 may be controlled by a physician manually or robotically. Manipulation of ultrasound transceiver 128 may take the form of activating the transceiver to probe or sweep a region of an airway adjacent to the target and/or the tool in search of either or both. Communication or feedback 1280 from the transceiver may take the form of ultrasound image input.

Control system 1000 (e.g. manipulator module 1012 and/or model-image bridging module 1010) may utilize communication 1280 to facilitate adjustment (e.g. fine-tuning) of the disposition of ultrasound transceiver 128 and/or tool 138. For some implementations, the operator may perform this adjustment (e.g. manually or semi-automatically)—e.g. control system 1000 displays an ultrasound image output from ultrasound transceiver 128 on screen 224. For some implementations, control system 1000 performs this adjustment automatically in response to communication 1280—e.g. via communication 1380.

It is to be noted that procedures using techniques other than a tool and an ultrasound transceiver may likewise benefit from a two (or more) catheter approach. Examples include but are not limited to using one or more catheters to advance a probe or transceiver for CT, optical coherence, infrared, confocal imaging, and/or other imaging devices or treatment modalities. In some implementations, a thermometer or infrared sensor may be inserted via a second (or additional) catheter to measure the temperature of tissue being treated by infrared, RF, or other heat-based therapy administered via a tool inserted through a first catheter. Such temperature monitoring may be particularly useful to ensure optimal temperature and rate of heating of the target tissue.

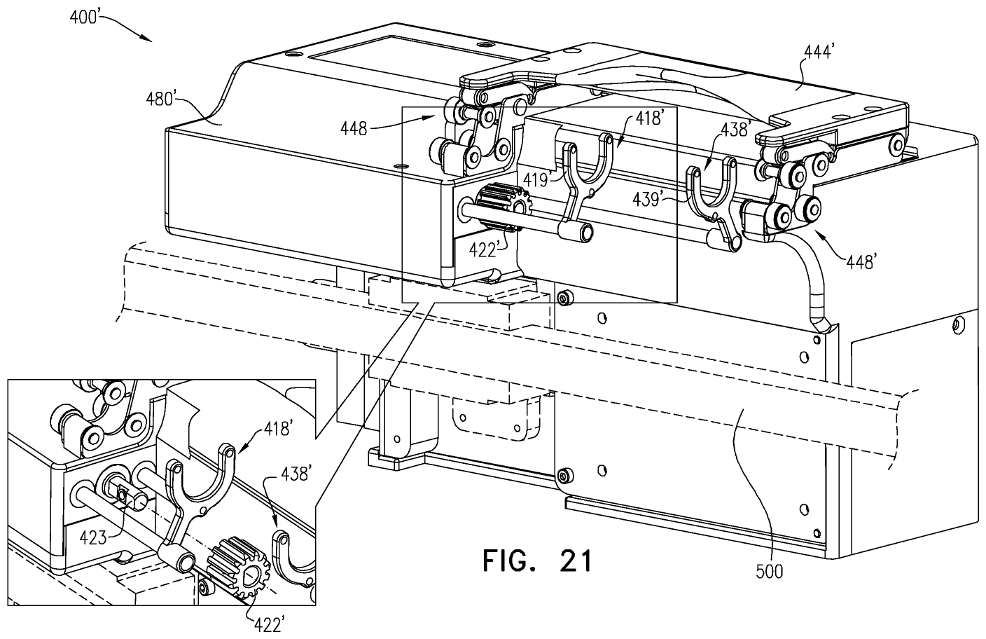

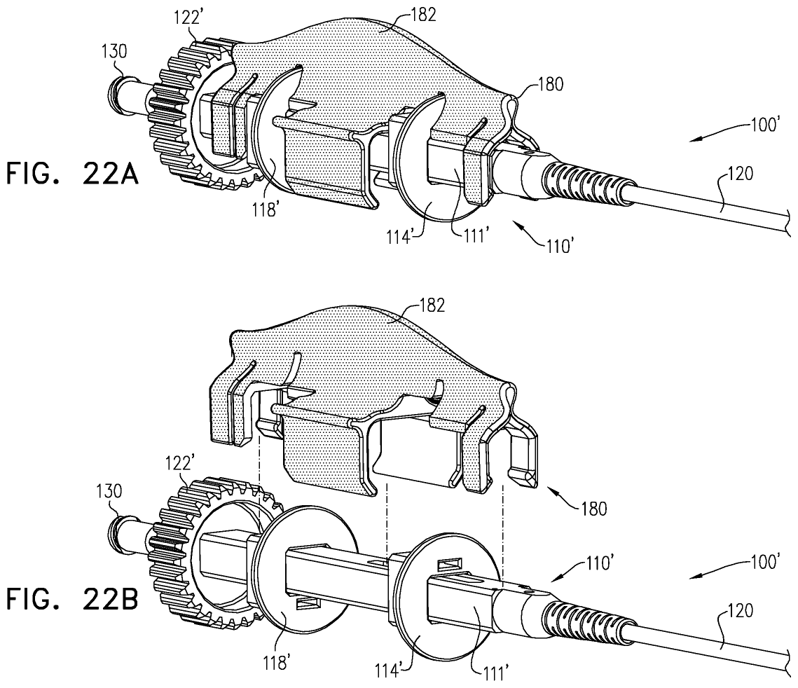

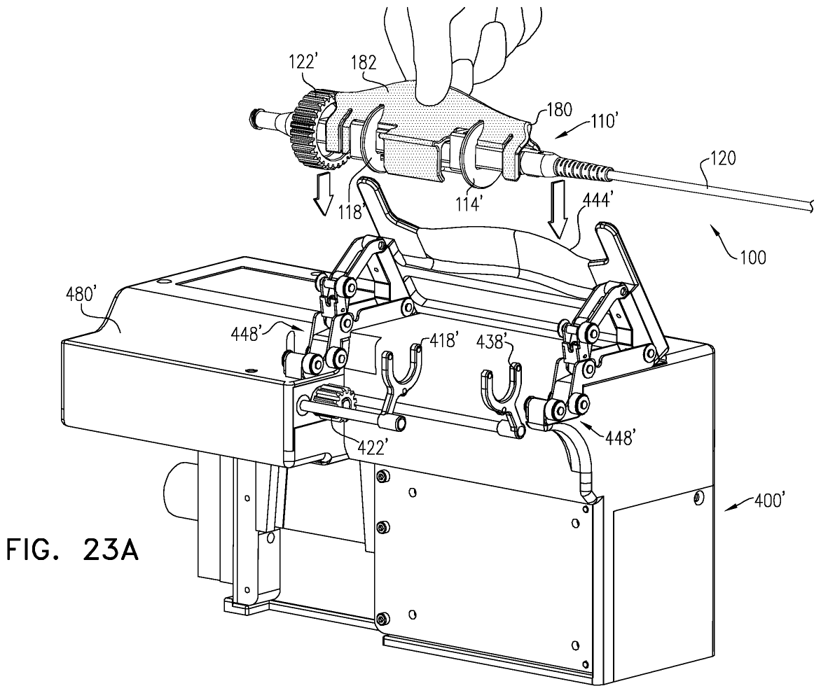

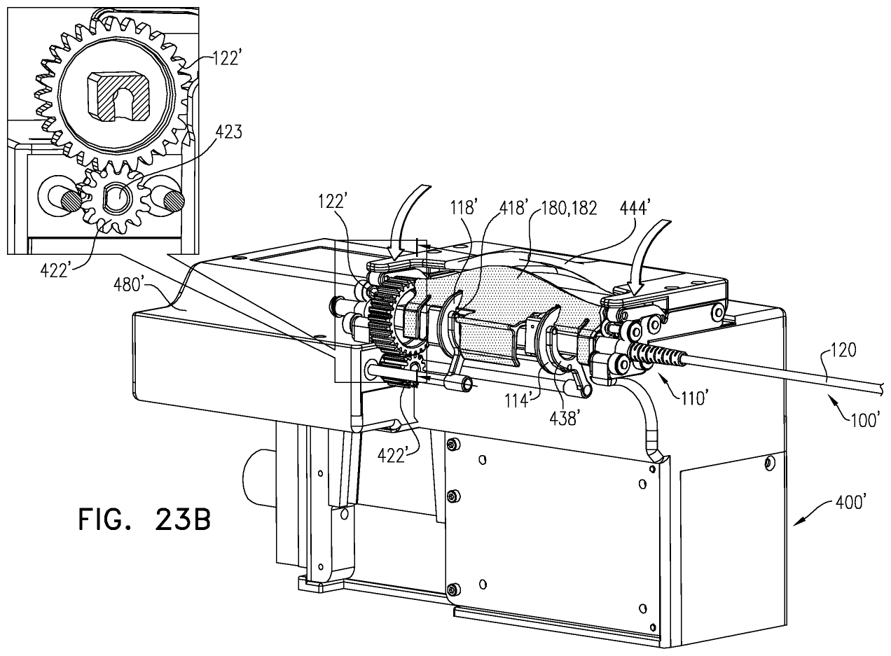

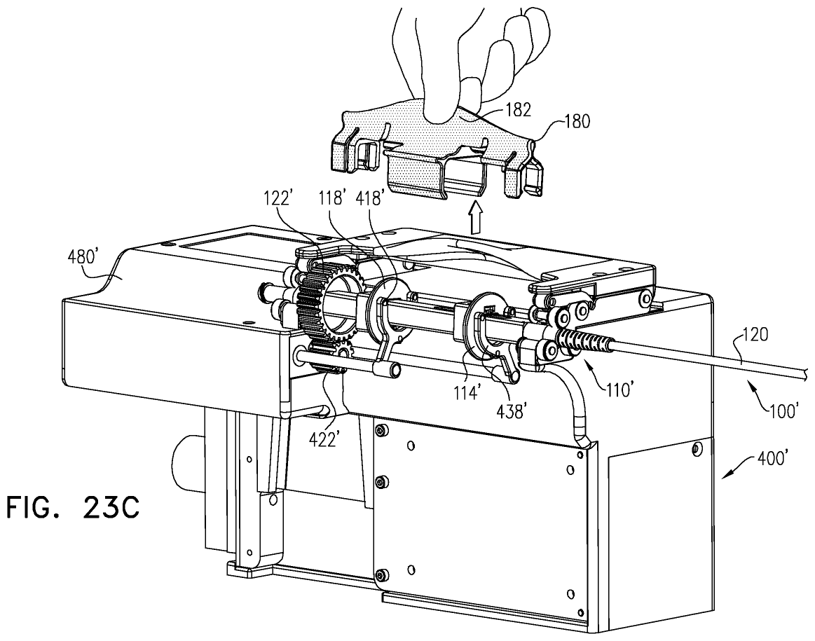

Reference is now made to FIGS. 21, 22A-B, and 23A-C, which are schematic illustrations of a steering manipulator 400′ and a catheter 100′ for use therewith, in accordance with some implementations. As noted hereinabove, the suffix ‘denotes that these are variants of, and can be substituted with, steering manipulator 400 and catheter 100, respectively—e.g. as components of system 50.

Steering manipulator 400’ has the same functionality, and has similar componentry, to that described for steering manipulator 400. However, the structure of steering manipulator 400′ is further optimized for usability and hygiene. For example, cradles 418′ and 438′ of steering manipulator 400′ are more minimalistic than the cradles shown for steering manipulator 400. For example, cradles 418′ and 438′ may be simple hooks (e.g. as shown) or other projections that define respective push-faces 419′ and 439′. This may advantageously facilitate decontamination of the cradles. Similarly, a unified closure 444′ allows both gates 448′ to be operated simultaneously. Furthermore, most of the other componentry (e.g. the actuators, motors, encoders, force sensors) of steering manipulator 400′ is closed (e.g. protected and/or sealed) inside a housing 480′. Each cradle may be connected to this componentry (e.g. to a respective actuator) via a stem that extends out of the housing, and is slid into and out of the housing in order to move its cradle.

Like steering manipulator 400, steering manipulator 400′ utilizes a manipulator gearwheel that becomes engaged with the head-gearwheel of the catheter (e.g. upon the catheter being loaded into the steering manipulator) in a manner that enables the steering manipulator to rotate the catheter by rotating the head of the catheter. For some implementations, and as shown (see, for example, FIG. 21), the manipulator-gearwheel utilized by steering manipulator 400′ is a single-use gearwheel 422′. The operator may mount single-use gearwheel 422′ onto a drive axle 423 that is operated by the motor of steering manipulator 400′, and may discard the single-use gearwheel after use (e.g. along with catheter 110′). Thus, like manipulator-gearwheel 422 of steering manipulator 400, single-use gearwheel 422′ is driven by a motor of the steering manipulator. However, utilization of a single-use gearwheel may be advantageously hygienic—e.g. because the relatively smooth drive axle may be easier to decontaminate than a gearwheel that has many notches.

Some of the ways in which catheter 100′, and in particular its head 110′, differs from catheter 100 are complementary to some of ways in which steering manipulator 400′ differs from steering manipulator 400′. For example, plungers 118′ 114′ of catheter 110′ may be flatter (e.g. more like a disk and less like a cylinder) (e.g. see FIG. 22B), and rather than becoming cradled within the cradles of the steering manipulator, they become positioned adjacent to the cradles (e.g. see FIGS. 23B-C). Whereas head-gearwheel 122 of catheter 100 is distal to plungers 118 and 114 (e.g. at a distal end of stem 111), head-gearwheel 122′ of catheter 100′ is proximal to plungers 118′ and 114′ (e.g. at a proximal end of stem 111′).

For some implementations, a holder 180 may be provided. Holder 180 is removably mounted on head 110′—e.g. by snap-fit. FIG. 22A shows holder 180 mounted on head 110′, and FIG. 22B shows the holder adjacent to the head. Holder 180 may be shaped to define a handle 182 that facilitates handling of head 110′ by a human operator. For example, handle 182 may facilitate the operator loading head into steering manipulator 400′ (e.g. see FIG. 23A).

Holder 180 may be mounted in a manner that inhibits linear sliding of plunger 118′ and/or plunger 114′ along stem 111′. For example, holder 180 may retain plunger 118′ at a first linear position along stem 111′ and/or retain plunger 114′ at a second linear position along the stem. These linear positions may be those suitable (e.g. optimal) for the loading of head 110′ into steering manipulator 400′. For example, holder 180 may ensure that, upon loading of head 110′ into steering manipulator 400′, plunger 114′ is positioned at (e.g. just proximal to) push-face 439, and plunger 118′ is positioned at (e.g. just proximal to) push-face 419′—e.g. in order that the steering manipulator can manipulate the steering region of catheter 100′ by pushing the push-faces proximally against the plungers.

FIG. 23A shows head 110′ being loaded into steering manipulator 400′ by an operator holding handle 182 of holder 180, and FIG. 23B shows gates 448′ having been subsequently closed. The removable mounting (e.g. the snap-fit) of holder 180 onto head 110′ allows the head to be easily removed (FIG. 23C), leaving catheter 100′ ready to be manipulated by steering manipulator 400′.

Holder 180, or a similar holder, may similarly be used with catheter 100, mutatis mutandis.

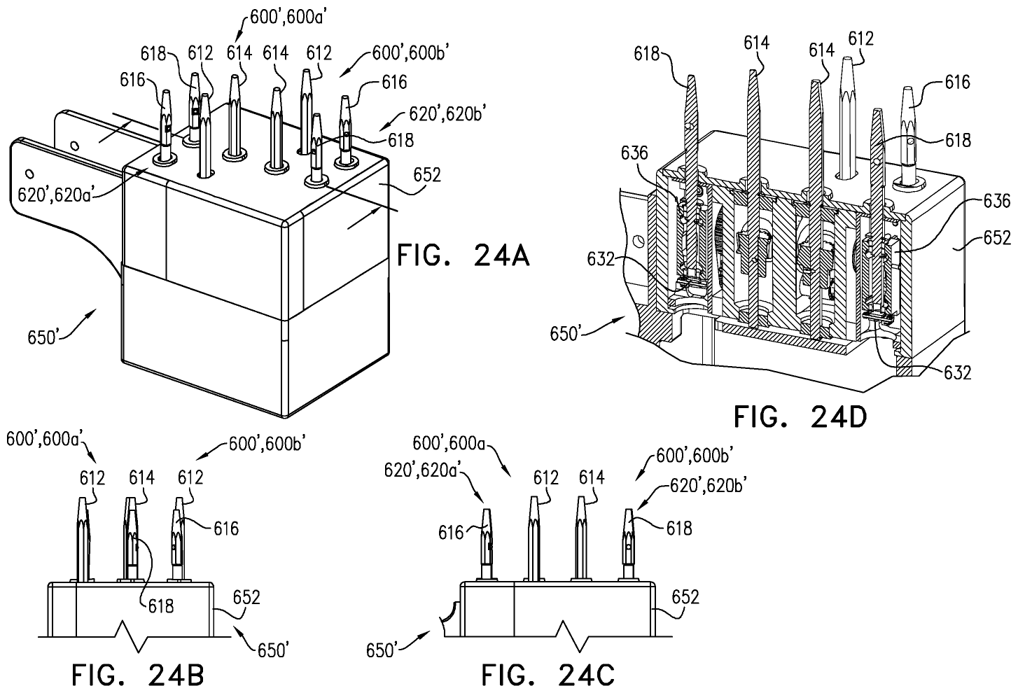

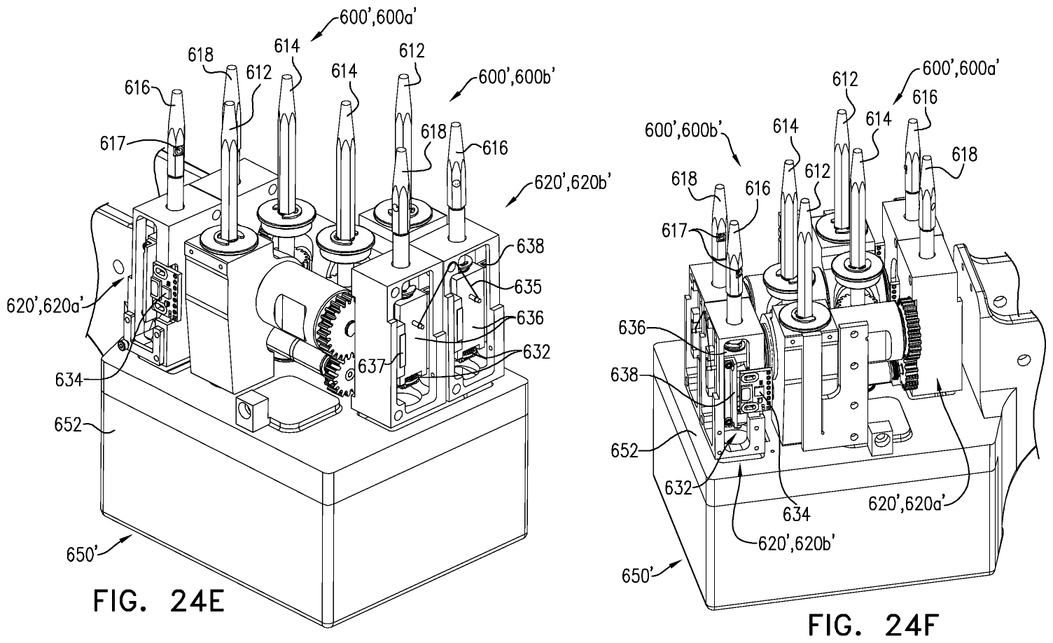

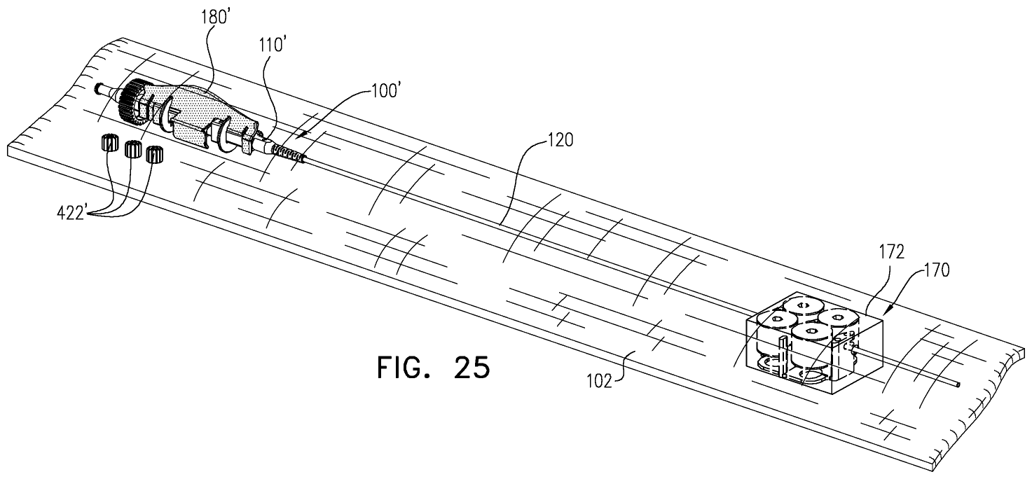

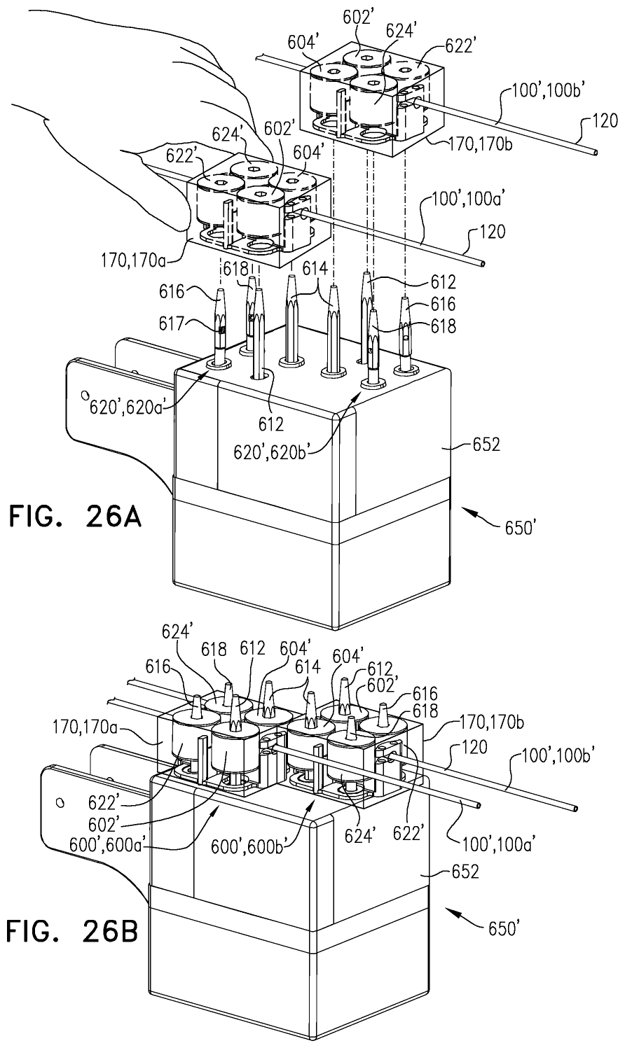

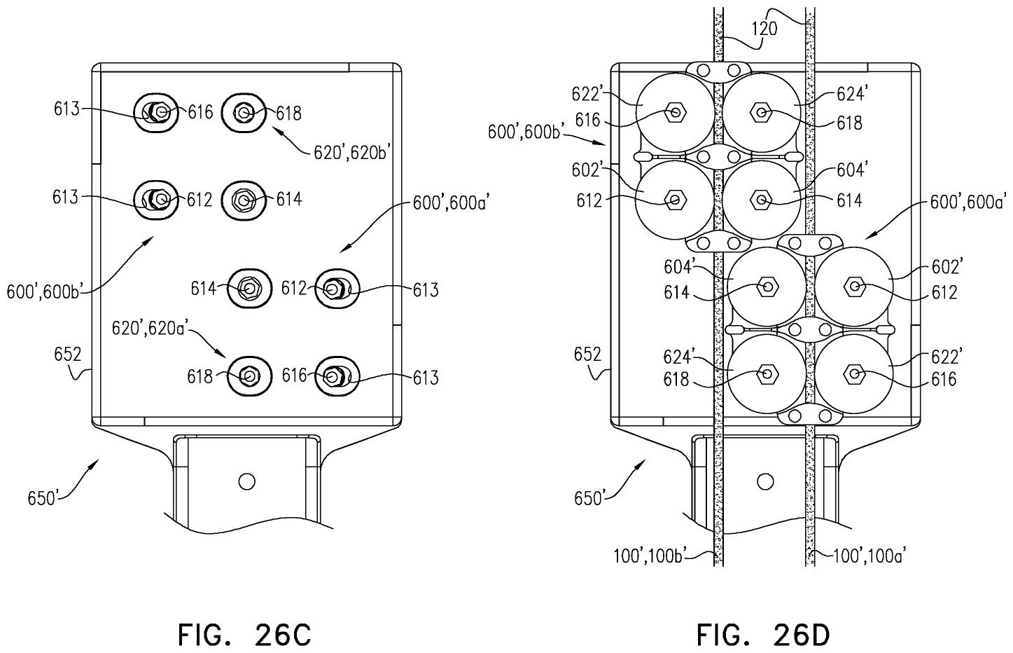

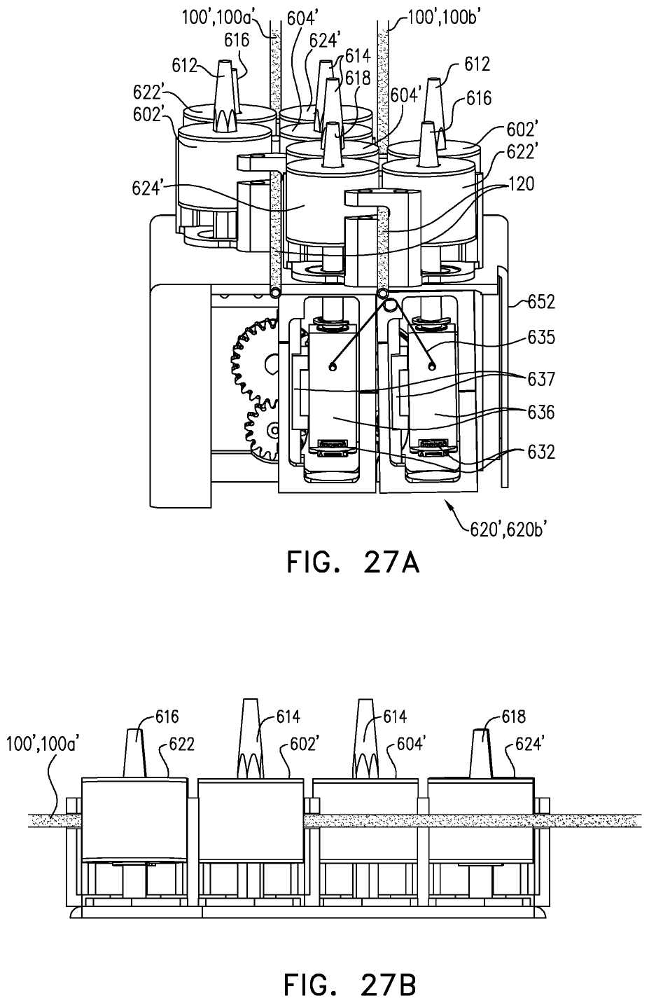

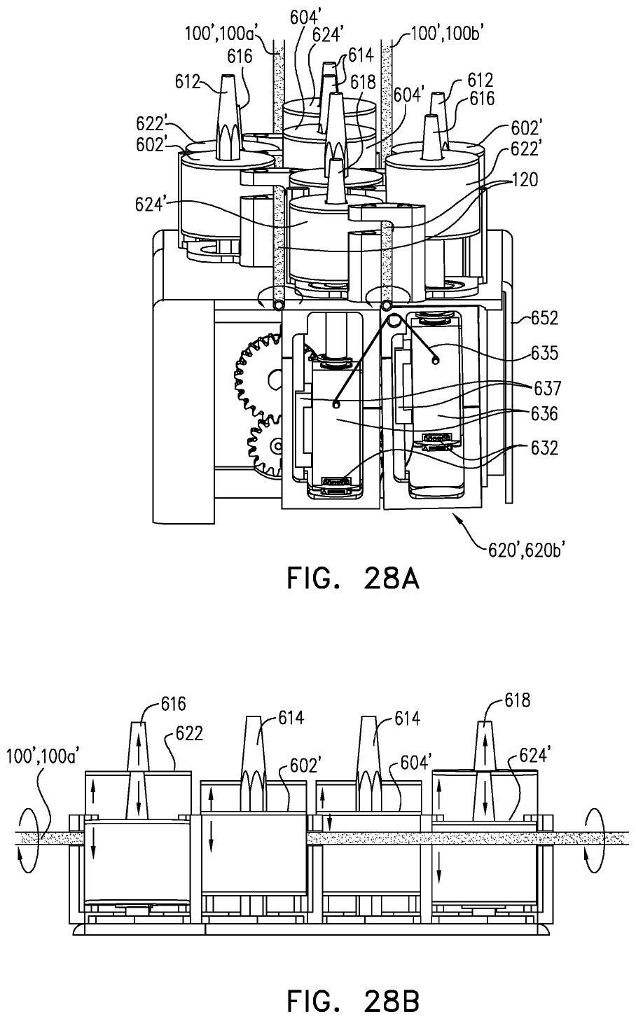

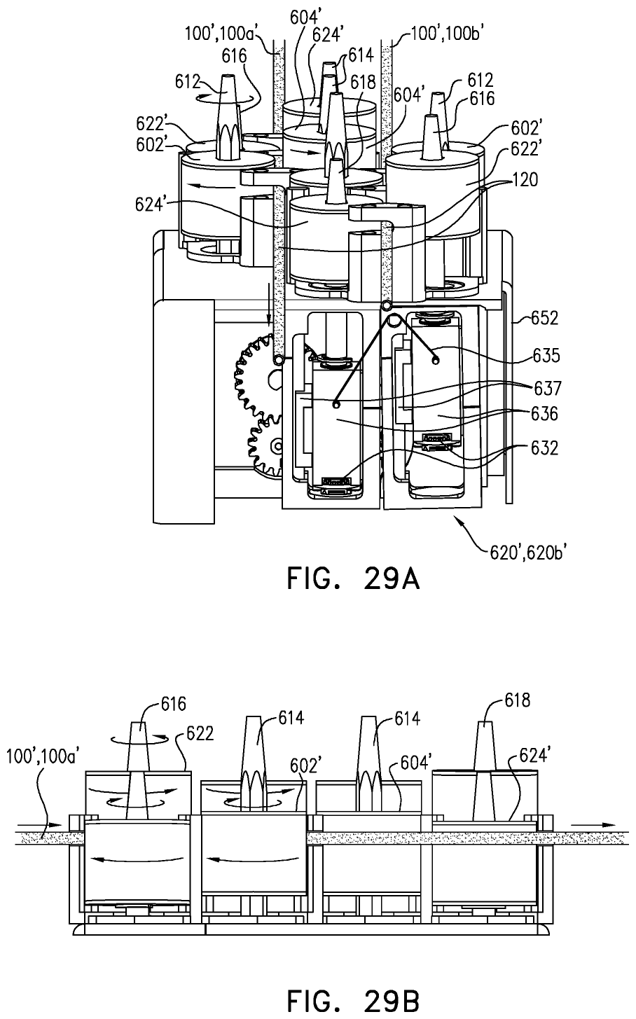

Reference is now further made to FIGS. 24A-F, 25, 26A-D, 27A-B, 28A-B, and 29A-B, which are schematic illustrations of, inter alia, an advancement unit 650′ and rollers 602′ and 604′, in accordance with some implementations. As noted hereinabove, the suffix ‘denotes that advancement unit 650’ and rollers 602′ and 604′ are variants of, and can be substituted with, advancement unit 650 and rollers 602 and 604, respectively—e.g. as components of system 50.

Advancement unit 650′ typically comprises two (or more) advancement manipulators 600′—e.g. a first advancement manipulator 600a′ for one catheter and a second advancement manipulator 600b′ for another catheter. Each advancement manipulator 600′ has the same general functionality, and has similar componentry, to that described for advancement manipulator 600. However, the structure of advancement manipulator 600′ is further optimized for usability and hygiene. In particular, rather than the rollers being (fixed or removable) components of the advancement manipulator (e.g. as described hereinabove for advancement manipulator 600), rollers 602′ and 604′ may be provided pre-coupled to tube 120 of catheter 100′—e.g. with the tube already disposed between the rollers. Thus, FIGS. 24A-F show advancement manipulators 600′ without rollers, but instead with motorized feed-axles 612 and 604 that are configured to receive rollers 602′ and 604′ as catheter 100′ is loaded into the manipulator assembly of the robot. For example, FIGS. 24A-C may show advancement manipulator 600′ as it appears within the manipulator structure (e.g. manipulator structure 300)—e.g. within the manipulator assembly (e.g. manipulator assembly 310).

For example, rollers 602′ and 604′ may be components of a feeder 170 (e.g. a roller assembly) that is mounted on tube 120, and that is configured (or configurable) to feed the tube through the feeder—e.g. in order to advance the tube into the subject (e.g. along the planned route) and, when feeding in reverse, to withdraw the tube from the subject. For example, the rollers may be mounted inside a housing 172 of feeder 170. The scope of the present disclosure includes implementations in which such a feeder is active (e.g. motorized and/or electronically connected to control system 1000)—e.g. similarly to as described for advancement manipulator 600, mutatis mutandis. However, in the example shown, feeder 170 is passive, and is driven by advancement manipulator 600′ which, itself, is motorized and electronically connected to control system 1000. For some implementations, roller assembly (e.g. feeder 170) may therefore be considered to serve as an adapter that operatively couples the advancement manipulator to the tube of the catheter.

Therefore, for some implementations, catheter 600′ may be considered to include tube 120, head 110′ at a proximal region of the tube, and feeder 600′ mounted on the tube. FIG. 25 shows an example implementation of this, in which the catheter 100′ is provided, with feeder 170 already mounted on tube 120. This pre-mounting may advantageously obviate the need to thread the distal end and/or steering region 140 of catheter 100′ into an advancement manipulator and/or between rollers. Moreover, this pre-mounting may advantageously obviate the need for the operator to directly handle tube 120 (or catheter 100′) at all. Alternatively or additionally to the pre-mounting of feeder 170, holder 180 may be pre-mounted on head 110′. This “catheter assembly” may be provided without the robot—e.g. as a single-use consumable, such as in a sterilized (or sterilizable) packaging 102, such as shown in FIG. 25.

For some implementations, and as shown, one or more single-use gearwheels 422′ may be provided with the catheter assembly (e.g. inside packaging 102), for use when loading head 110′ of catheter 100′ into steering manipulator 400′.

Advancement unit 650′ advancement manipulators 600′ may comprise one or more sensors 620′, which may be considered to be a variant of sensor 620 and/or of sensor 610 described hereinabove. Each sensor 620′ is configured similarly to the motorized portion of the advancement manipulator—i.e. comprising a pair of axles configured to receive a pair of rollers of feeder 170 as catheter 100′ is loaded into the manipulator assembly. However, rather than being motorized, axles 616 and 618 of sensor 620′ are configured to move passively in response to movement of tube 120, such that the movement of the axles serves as a proxy for movement of tube 120—e.g. as described in more detail hereinbelow. The respective rollers, 622′ and 624′, which serve as components of sensor 620′, may therefore be considered variants of rider 622, and may therefore be considered to be “rider rollers”. Similarly, axles 616 and 618 may be considered to be “rider axles”. Conversely, motorized axles 612 and 614 and their corresponding rollers 602′ and 614′ may be considered to be “feed axles” and “feed rollers”, respectively.

For some implementations in which advancement unit 650′ comprises two advancement manipulators 600′, the advancement manipulators may have opposite orientations. For example, one advancement manipulator may have its rider axles and rider rollers proximal from its feed axles and feed rollers (e.g. as shown for advancement manipulator 600a′), while another advancement manipulator may have its rider axles and rider rollers distal to its feed axles and feed rollers (e.g. as shown for advancement manipulator 600b′). For some such implementations, and as shown, feeder 170 is generic with respect to these orientations, such that any given catheter 110′ may be loaded onto either advancement manipulator. For example, each roller of feeder 170 may be capable of being mounted onto a feed axle or a rider axle—e.g. all of the rollers may be identical to each other. Hence, in FIG. 25, which shows catheter 110′ prior to its loading onto an advancement manipulator, the rollers of feeder 170 are labeled as generic rollers 601.

For some implementations in which advancement unit 650′ comprises two advancement manipulators 600′, although catheters 100a′ and 100b′ are disposed side-by-side, the advancement manipulators may be arranged in a manner that allows advancement unit 650′ to be narrower (and allows the catheters to be closer to each other) than would be possible in a side-by-side arrangement of the advancement manipulators. For example, and as shown, a staggered arrangement in which axles 614 and 618 of both advancement manipulators are disposed along a common medial line allows the advancement unit to have the width of only three axles/rollers, rather than four (and for catheters 100a′ and 100b′ to be separated by approximately the width of one roller, rather than two).

FIGS. 26A-B shows two catheters 110′ (i.e. their feeders 170) being loaded onto advancement unit 650′ (e.g. advancement manipulators 600′ thereof), thereby designating one of the catheters as catheter 100a′, and one as 100b′. Although not shown in FIGS. 26A-B, this loading may be accompanied by loading of heads 110′ of the catheters into corresponding steering manipulators 400′. For example, one catheter 100′ may be loaded into both the steering manipulator and the advancement manipulator of one manipulator assembly, and the other catheter may then be loaded into both the steering manipulator and the advancement manipulator of the other manipulator assembly. Alternatively, the heads of both catheters may be loaded into the steering manipulators, followed by the loading of the feeders of both catheters into the advancement manipulators. Alternatively, the feeders of both catheters may be loaded into the advancement manipulators, followed by the loading of the heads of both catheters into the steering manipulators.

For some implementations, and as shown, this loading involves mounting the rollers of feeder 170 onto the axles of advancement manipulator 600′, thereby designating the previously-generic rollers as feed rollers and rider rollers. For example, housing 172 of feeder 170, in which the rollers are mounted, may have apertures via which the axles may be inserted into the rollers. As noted hereinabove, this may advantageously eliminate the need for threading the end of tube 120 into an advancement manipulator. For example, feeder 170 may simply be clicked into place.

FIGS. 26C and 26D are top-views of advancement unit 650′ before (FIG. 26C) and after (FIG. 26D) feeders 170 have been loaded. For the sake of clarity, housings 172 of feeders 170 are not shown in FIG. 26B, 26D, 27A-B, 28A-B, or 29A-B.

One or more of the feed rollers, and/or one or more of the rider rollers, may be configured to be pressed into engagement with tube 120. For example, feeder 170 may have a rest state in which at least one of its rollers is disengaged from tube 120—e.g. such that rotation of the roller would not feed the tube through the feeder. For example, advancement manipulator 600′ may be configured to press the roller(s) into engagement with tube 120.

This pressing may occur upon loading of feeder 170 onto advancement manipulator 600′—e.g. as an intrinsic effect of the rollers being mounted onto the axles. For example, for the feed axles and/or for the rider axles, at least one axle of each pair may be biased (e.g. spring-loaded) toward each other. In FIG. 26C (i.e. prior to loading), for each advancement manipulator, feed axle 612 and rider axle 616 are disposed closer to feed axle 614 and 618, respectively, compared to in FIG. 26D (i.e. after loading), illustrating such biasing, which the mounting of the rollers opposes. Note that feed axles 612 and rider axle 616 are disposed through enlarged (e.g. oblong) apertures 613 in the housing of advancement manipulator, which allow for the movement of these axles.

For other implementations, the pressing of the rollers into engagement with tube 120 is a discrete function and/or step. For example, rather than the axles being spring-loaded, they may be moved manually (e.g. using a lever) or electromechanically (e.g. under control of control system 1000).

Irrespective of the manner in which advancement manipulator presses the rollers into engagement with tube 120, it may advantageously protect the tube from plastic deformations that may otherwise occur were the rollers to be maintained in engagement with (i.e. pressing against) the tube—e.g. during storage.

In order for advancement manipulator 600′ to drive feed rollers 602′ and 604′, the mounting of each feed roller onto its feed axle may rotationally lock the feed roller onto its feed axle. Rotational locking may be provided by, for example, complimentary splining or keying of the feed axle and the feed roller. In the example shown, this is achieved by the feed axle having a hexagonal form factor.

FIGS. 27A-B, 28A-B, and 29A-B are schematic illustrations of end-views (A) and side-views (B) of catheters 100a′ and 100b′ with their respective feeders 170 loaded into advancement manipulators 600′ of advancement unit 650′, in accordance with some implementations. FIGS. 27A-B show an illustrative example of a state immediately after the loading of feeders 170, FIGS. 28A-B show an illustrative example of catheters 100a′ and 100b′ having been subsequently rotated (e.g. by the respective steering manipulator 400′), and FIGS. 29A-B show an illustrative example of catheter 100a′ having been subsequently advanced distally (e.g. by the respective advancement manipulator 600′).

As described hereinabove, advancement (and retraction) of catheter 100′ is performed by advancement manipulator 600′ feeding tube 120 of the catheter, whereas rotation of the catheter is typically performed by steering manipulator 400′ rotating head 110′ of the catheter. Advancement manipulator 600 is described hereinabove as having linear treads that grip tube 120 sufficiently to feed the tube while nonetheless allowing rotation of the tube (induced by the steering manipulator) by allowing rotational slippage of the tube between the feed rollers. In contrast, advancement manipulator 600′, in cooperation with feeder 170, advantageously allows rotation of tube 120 without requiring slippage between the tube and feed rollers. Feed rollers 602′ and 604′ are axially translatable along feed axles 612 and 614—i.e. can be slid along the feed axles by rotation of tube 120. Thus, rotation of tube 120 in a first rotational direction slides feed roller 602′ up feed axle 612 and feed roller 604′ down feed axle 614 (e.g. as shown in FIGS. 28A-B for catheter 100b′), and rotation of the tube in a second, opposite, rotational direction slides feed roller 602′ down feed axle 612 and feed roller 604′ up feed axle 614 (e.g. as shown in FIGS. 28A-B for catheter 100a′). That is, each feed roller has a feed-roller axis about which it rotates when feeding tube 120, and along which it translates responsively to rotation of tube 120. The feed-roller axis may be defined by the respective axle of the advancement manipulator—e.g. the axle lies on the feed-roller axis. As shown, the feed-roller axes of a given advancement manipulator 600′ may be parallel with each other.

Note that feed axles 612 and 614 remain in the same axial position in FIGS. 27A-B and 28A-B, illustrating that the feed axles typically do not translate with their feed rollers. For example, they may be axially fixed in order to be drivable by the motor of the advancement manipulator.

Thus, feed rollers 602′ and 604′ actively rotate in order to linearly feed tube 120, and passively translate in order to accommodate rotation of the tube.

Each sensor 620′ is configured to detect rotation and/or advancement of its corresponding catheter 100′ (e.g. the tube 120 of the catheter), such as via one or more electromechanical encoders. Rotation of the catheter may be detected by sensing translation of roller 622′ and/or roller 624′ along their axes of rotation. This may be achieved by sensing translation of axle 616 and/or 618—e.g. by one or more linear encoders 634. Advancement of the catheter may be detected by sensing rotation of roller 622′ and/or roller 624′. This may be achieved by sensing rotation of axle 616 and/or 618—e.g. by one or more rotary encoders 632. That is, each rider roller has a rider-roller axis about which it rotates responsively to advancement of tube 120, and along which it translates responsively to rotation of tube 120. The rider-roller axis may be defined by the respective axle of the sensor—e.g. the axle lies on the rider-roller axis. As shown, the rider-roller axes of a given sensor 620′ may be parallel with each other.

For implementations, such as that shown, in which rotation and translation of rider rollers 622′ and 624′ is detected via rotation and translation of their rider axles 616 and 618, the mounting of each rider roller onto its rider axle may rotationally and axially lock the rider roller onto its rider axle, such that the rider roller and the rider axle move in unison-both axially and rotationally. Axial locking may be provided by, for example, a ball detent 617 or similar (e.g. a snap fit) on each rider axle that latches onto the rider roller upon mounting. Rotational locking may be provided by, for example, complimentary splining or keying of the rider axle and the rider roller. In the example shown, this takes the form of the rider axles having a hexagonal form factor.

For some implementations, and as shown, each rider axle (e.g. its base) is rotatably mounted within a mount 636 that itself is linearly slidably mounted within housing 652 of advancement unit 650′, such as via a rail 637. Rotary encoder 632 may detect rotation of the rider axle relative to mount 636. Rotary encoder 632 may be mounted on mount 636—e.g. such that it rides with the mount as the mount, the rider axle, and the rider roller translate together along the rider-roller axis. Linear encoder 634 may detect translation of the rider axle (and thereby of the rider roller) by detecting translation of mount 636. For example, a readhead of linear encoder 634 may be fixedly mounted to housing 652, and may be paired with a scale 638 that is fixedly mounted to mount 636. Note that rider axles 616 and 618 axially translate along with their rider rollers in the transition from FIGS. 27A-B to FIGS. 28A-B.

For some implementations, each sensor 620′ comprises a linkage 635 that inversely links the two rider axles of the sensor (e.g. by linking the two mounts 636 of the sensor). In this context, “inversely links” means that the linkage causes the translation of each rider axle along its rider-roller axis to be equal and opposite to that of the other rider axle. Thus, although, as described hereinabove, rotation of the catheter 100′ causes one rider axle of the sensor to move upward and one to move downward, linkage 635 may enforce this behavior more strictly, thereby balancing the movement of the two rider axles. This may advantageously improve the accuracy of the detection of catheter rotation by sensor 620′—e.g. by mechanically averaging the movement of the rider axles. In FIGS. 27A, 28A, and 29A, part of housing 652 is cut away to show linkage 635 of sensor 620b′, which is also visible in FIG. 24E. In the example shown, linkage 635 is a tether that extends over a bearing (e.g. in a pulley arrangement), but the scope of the present disclosure includes other linkages, such as mechanical linkages comprising links and joints, and/or enmeshed gears.

Utilizing rider rollers that are independent of the feed rollers may advantageously enhance the accuracy of both sensing of catheter advancement and sensing of catheter rotation. Regarding sensing of catheter advancement, tube 120 may be less likely to slip with respect to the rider rollers than with respect to the feed rollers—at least in part because the rider axles and rider rollers can be configured to exhibit little resistance to being passively rotated by the tube as it is fed through the feeder. In contrast, the motorized feed axles and feed rollers necessarily resist such passive rotation in order to perform their function of feeding the tube of the catheter. Regarding sensing of catheter rotation, tube 120 may be less likely to slip with respect to the rider rollers than with respect to the feed rollers—at least in part because the rider axles and rider rollers can be configured to exhibit little resistance to being passively translated by the tube as it is rotated within the feeder. In contrast, and as described hereinabove, passive translation of each feed roller occurs by the feed roller sliding along its feed axle, which may be mounted in a fixed axial position in order to be motorized. Although this sliding may facilitate rotation of tube 120 between the feed axles (e.g. as described hereinabove), friction between each feed roller and its feed axle may nonetheless cause some slippage of the tube between the feed rollers. This advantage of independent rider rollers is illustrated in the transition from FIGS. 27A-B to FIGS. 28A-B, whereby feed rollers 602′ and 604′ axially translate by a shorter distance than rider rollers 622′ and 624′ in response to rotation of tube 120.

In the example shown, sensor 620′ senses rotation and advancement of tube 120 via both of its rider axles—e.g. comprises two rotary encoders and two linear encoders. However, for some implementations, sensor 620′ senses rotation and/or advancement of tube 120 via only one of its rider axles—e.g. with the other of the rider axles simply providing a reference force. For example, sensor 620′ may detect rotation and advancement of tube 120 via one of its rider axles, with the other rider axle being solely mechanical. Alternatively, sensor 620′ may detect rotation of tube 120 via one of its rider axles and advancement of the tube via the other of its rider axles.

It is to be noted that the apparatus and techniques described with respect to advancement unit 650′, advancement manipulator 600′, and sensor 620′ may be applied to other systems, e.g. for feeding and/or sensing advancement and rotation of other catheters, or other elongate members such as tubes or shafts. Furthermore, this applies not only to systems in which the advancement the rollers are separate/separable from their axles like those of advancement unit 650′ (e.g. not only to systems in which a separate/disposable adapter or feeder is used with the axles). There is therefore provided, in accordance with some implementations, apparatus for use with an elongate member, the apparatus comprising an advancement unit that comprises: (1) a feed axle that lies on a feed-axle axis; (2) a motor, operatively coupled to the feed axle such that operation of the motor rotates the feed axle about the feed-axle axis; (3) a rider axle that lies on a rider-axle axis; and/or (4) one or more sensors, operatively coupled to the rider axle in a manner that detects: (i) rotation of the rider axle about the rider-axle axis, and/or (ii) translation of the rider axle along the rider-axle axis. The advancement unit may be configured to receive the elongate member in a manner in which: (1) operation of the motor feeds the elongate member through the advancement unit via rotation of the feed axle about the feed-axle axis, such that the elongate member rotates the rider axle about the rider-axle axis, and/or (2) rotation of the elongate member within the advancement unit translates the rider axle along the rider axis.

Advantages of the systems and methods described herewithin include the positioning of catheter(s) specifically in relation to the actual airways of the subject, and route(s) through the airways, as opposed to, for example, positioning relative to a mere model of the airways, or positioning in three-dimensional space (e.g. in relation to emitters and/or sensors outside of the subject, such as using fluoroscopy and/or (electro) magnetic navigation). For example, the systems and methods described herein provide navigation that is relatively insensitive to respiratory expansion and contraction of the subject's lungs, and differences in position and posture of the subject.

A further advantage of methods described herein relates to the distal airways, where most lesions are located, and in which small discrepancies between the model and the lung may tend to cause errors in catheter guidance. As described hereinabove, superimposing the planned path from the model onto an image acquired by a camera in the airway, and the ability to update the model with data acquired intra-procedurally, provides the physician or other user with greater confidence regarding the direction of catheter advancement.

The systems and techniques described herein may be supplemented by electromagnetic-based macro-scale positioning—e.g. as described in International Patent Application (PCT) Publication WO 2023/017460.

The systems (and/or components thereof) and techniques described herein may be used in combination with, and/or to facilitate, any of those described in any of the following references, each of which is incorporated herein by reference in its entirety:
Provisional U.S. Patent Application 63/231,895 to Shapira et al., filed 11 Aug. 2021, and titled “Techniques for accessing lung tissue”
Provisional U.S. Patent Application 63/247,424 to Shapira et al., filed 23 Sep. 2021, and titled “Tubular assembly for bronchoscopic procedures”
International Patent Application PCT/IB2022/057505 to Shapira et al., filed Aug. 11, 2022, and titled “Two-pronged approach for bronchoscopy,” which published as WO 2023/017460
International Patent Application PCT/IB2022/058307 to Shapira et al., filed Sep. 4, 2022, and titled “Steerable tubular assembly for bronchoscopic procedures,” which published as WO 2023/047219

For some implementations, and as shown, the data-processing system is, or is a component of, a discrete (e.g. purpose-made) device. For some implementations, the data-processing system is a general-purpose data-processing system (e.g. a processor of a general-purpose computer) programmed to run the program.

In the present disclosure, the term data-processing system may refer to, be part of, or include an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor (shared, dedicated, group) that executes code; memory (shared, dedicated, or group) that stores code executed by a processor; other suitable hardware components, such as optical, magnetic, or solid state drives, that provide the described functionality; or a combination of some or all of the above, such as in a system-on-chip. The term code, as used above, may include software, firmware, and/or microcode, and may refer to programs, routines, algorithms, functions, classes, and/or objects. The term shared processor encompasses a single processor that executes some or all code from multiple modules. The term group processor encompasses a processor that, in combination with additional circuitry (e.g. processors), executes some or all code from one or more modules. The term shared memory encompasses a single memory that stores some or all code from multiple modules. The term group memory encompasses a memory that, in combination with additional memories, stores some or all code from one or more modules. The term memory may be subset of the term computer-readable medium. The term computer-readable medium does not encompass transitory electrical and electromagnetic signals propagating through a medium, and may therefore be considered tangible and non-transitory. Non-limiting examples of a non-transitory tangible computer readable medium include nonvolatile memory, volatile memory, magnetic storage, and optical storage.

The various systems, devices, apparatuses, etc. in this disclosure can be sterilized (e.g. with heat, radiation, ethylene oxide, hydrogen peroxide, etc.) to ensure they are safe for use with patients, and the methods herein can comprise such sterilization of the associated system, device, apparatus, etc. Furthermore, the scope of the present disclosure includes, for some implementations, sterilizing one or more of any of the various systems, devices, apparatuses, etc. in this disclosure.

Any of the techniques, methods, operations, steps, etc. described or suggested herein can be performed on a living animal (e.g. human, other mammal, etc.) or on a non-living simulation, such as a cadaver, a cadaver heart, an anthropomorphic ghost, and/or a simulator device (which may include computerized and/or physical representations of body parts, tissue, etc.).

Although the operations of some of the disclosed methods are described in a particular, sequential order for convenient presentation, it should be understood that this manner of description encompasses rearrangement, unless a particular ordering is required by specific language set forth herein. For example, operations described sequentially can in some cases be rearranged or performed concurrently. Moreover, for the sake of simplicity, the attached figures may not show the various ways in which the disclosed systems, apparatuses, devices, methods, etc. can be used in conjunction with other systems, apparatuses, devices, methods, etc.

Example Implementations (some non-limiting examples of the concepts herein are recited below):

Example 1. A system, comprising:
a catheter, comprising:
   a head, and
   a tube, having a steering region at a distal portion thereof; and
a robot, comprising:
   a manipulator structure that defines an advancement path, and comprises:
      a manipulator assembly, comprising:
         a steering manipulator, slidable along the advancement path, configured to receive the head in a manner that operatively couples the steering manipulator to the steering region such that a curvature of the steering region is adjustable by the steering manipulator manipulating the head, and
         an advancement manipulator, configured to receive the tube such that operation of the advancement manipulator draws the steering manipulator along the advancement path by feeding the tube though the advancement manipulator.

Example 2. The system according to example 1, wherein the catheter is sterilized.

Example 3. The system according to any one of examples 1-2, wherein the manipulator structure further comprises a sensor, the sensor positioned and configured to:
sense linear advancement of the catheter; and
provide an advancement output indicative of the sensed linear advancement.

Example 4. The system according to any one of examples 1-3, wherein the manipulator structure is configured such that the advancement path is substantially horizontal.

Example 5. The system according to any one of examples 1-4, wherein the manipulator structure is configured such that the advancement path is substantially vertical.

Example 6. The system according to any one of examples 1-5, wherein the manipulator structure is arranged such that drawing of the steering manipulator along the advancement path by the advancement manipulator draws the steering manipulator closer to the advancement manipulator.

Example 7. The system according to any one of examples 1-6, wherein the robot further comprises a robotic control system.

Example 8. The system according to any one of examples 1-7, wherein the manipulator structure is configured such that the advancement path is substantially straight between the steering manipulator and the advancement manipulator.

Example 9. The system according to any one of examples 1-8, wherein the advancement manipulator is configured to rotate the catheter by rotating the tube.

Example 10. The system according to any one of examples 1-9, wherein the advancement manipulator is configured to allow rotational slipping of the tube.

Example 11. The system according to any one of examples 1-9, wherein the advancement manipulator is configured to disallow rotational slipping of the tube.

Example 12. The system according to any one of examples 1-11, wherein the steering manipulator is biased to retreat proximally along the advancement path.

Example 13. The system according to example 12, wherein the steering manipulator is biased to retreat proximally along the advancement path by the advancement path sloping downward proximally.

Example 14. The system according to example 12, wherein the steering manipulator is biased to retreat proximally along the advancement path by a pulley attached to a slidable mounting.

Example 15. The system according to example 12, wherein the steering manipulator is biased to retreat proximally along the advancement path by a spring.

Example 16. The system according to example 12, wherein the steering manipulator is biased to retreat proximally along the advancement path by gravitational pull.

Example 17. The system according to any one of examples 1-16, wherein the catheter further comprises:
a first wire, and a second wire, each of the first wire and the second wire extending from the steering region proximally along the tube; and
wherein the head further comprises:
   a stem,
   a first plunger, operatively coupled to the steering region by being attached to
the first wire, and mounted on the stem to be slidable linearly along the stem, and
   a second plunger, operatively coupled to the steering region by being attached to the second wire, and mounted on the stem to be slidable linearly along the stem independently of the first plunger.

Example 18. The system according to example 17, wherein the steering manipulator is configured to receive the head in a manner that operatively couples the steering manipulator to the first plunger and the second plunger in a manner that configures the steering manipulator to control sliding of the first plunger and the second plunger linearly along the stem.

Example 19. The system according to example 17, wherein each of the first plunger and the second plunger is slidably coupled to the stem independently of the other plunger.

Example 20. The system according to example 17, wherein application of a sliding force to either plunger slides the plunger in a first direction along the stem in a manner that adjusts a curvature of the steering region by applying tension to the respective wire, and releasing the sliding force allows the respective wire to relax, by the wire responsively pulling the plunger in a reverse direction along the stem.

Example 21. The system according to example 17, wherein application of a force to the head adjusts a curvature of the steering region by applying tension to one of the first wire or the second wire, and concurrent tensioning of both wires adjusts a stiffness of the steering region such that the steering region maintains a specific curvature.

Example 22. The system according to example 17, wherein the first wire is operatively coupled to the steering region in a manner that causes deflection of the steering region in a first direction upon tensioning of the first wire, and the second wire is operatively coupled to the steering region in a manner that causes deflection of the steering region in a second direction upon tensioning of the second wire, the second direction being opposite to the first direction.

Example 23. The system according to example 17, wherein the steering manipulator is configured to control, via sliding of the first plunger and the second plunger linearly along the stem, (i) a curvature of the steering region, and (ii) a stiffness of the steering region.

Example 24. The system according to example 17, wherein the system is configured such that loading the head into the steering manipulator enables the steering manipulator to rotate, bend, and straighten the steering region.

Example 25. The system according to example 17, wherein, for each of the plungers, the plunger is mounted on the stem in a manner in which, while the head is not engaged by the steering manipulator, the plunger is slidable freely along at least part of the stem.

Example 26. The system according to example 17, wherein the first plunger is configured to apply tension to the first wire to control a curvature of the steering region.

Example 27. The system according to example 17, wherein the second plunger is configured to apply tension to the second wire to control a curvature of the steering region.

Example 28. The system according to example 17, wherein the steering manipulator is configured to receive the head in a manner that enables the steering manipulator to, while remaining operatively coupled to the first plunger and the second plunger, rotate the steering region by rotating the head.

Example 29. The system according to example 28, wherein the steering manipulator comprises a motor configured to rotate the steering region by rotating the head while the head is loaded within the steering manipulator.

Example 30. The system according to example 29, wherein:
the motor is operatively coupled to a drive axle,
the head further comprises a head gearwheel, coupled to the stem, and
the system further comprises a single-use gearwheel, configured to be temporarily mounted on the drive axle such that loading the head into the steering manipulator operatively couples the motor to the head gearwheel via the drive axle and the single-use gearwheel, the single-use gearwheel being discardable with the catheter after use.

Example 31. The system according to example 17, wherein:
the catheter is a first catheter,
the steering manipulator is a first steering manipulator,
the system further comprises a second catheter and a second steering manipulator configured to engage a head of the second catheter in a manner that configures the steering manipulator to manipulate a steering region of the second catheter, and
the system further comprises a robotic control system, electronically couplable to both the first steering manipulator and the second steering manipulator in a manner that enables the robotic control system to control both the first catheter and the second catheter.

Example 32. The system according to example 17, wherein the first plunger and the second plunger and the stem are complementarily shaped to rotationally lock the first plunger and the second plunger to the stem.

Example 33. The system according to example 32, wherein the complementary shaping defines a keyed joint.

Example 34. The system according to example 32, wherein the stem has a noncircular outer cross-section, and each of the first plunger and the second plunger has a complementary noncircular inner cross-section, thereby rotationally locking the first plunger and the second plunger to the stem.

Example 35. The system according to example 32, wherein the steering manipulator comprises:
a first cradle, configured to, upon the steering manipulator receiving the head, cradle the first plunger in a manner that operatively couples the steering manipulator to the first plunger while allowing the first plunger to slip rotationally within the first cradle, and
a second cradle, configured to, upon the steering manipulator receiving the head, cradle the second plunger in a manner that operatively couples the steering manipulator to the second plunger while allowing the second plunger to slip rotationally within the second cradle.

Example 36. The system according to example 17, further comprising a track on which the steering manipulator is slidably mounted.

Example 37. The system according to example 36, wherein the slidable mounting of the steering manipulator on the track enables distal advancement and proximal retraction of the catheter.

Example 38. The system according to example 17, wherein the first wire is operatively coupled to the steering region in a force-multiplication arrangement configured to increase a mechanical advantage of the first wire on the steering region.

Example 39. The system according to example 38, wherein the second wire is operatively coupled to the steering region in a second force-multiplication arrangement configured to increase a mechanical advantage of the second wire on the steering region.

Example 40. The system according to any one of examples 1-39, wherein the manipulator structure comprises a mount from which the steering manipulator hangs.

Example 41. The system according to example 40, wherein the manipulator structure is configured such that operation of the advancement manipulator draws the steering manipulator downward along the advancement path by feeding the tube though the advancement manipulator.

Example 42. The system according to example 40, wherein the manipulator structure comprises a winch configured to lift the steering manipulator upwards in retreat along the advancement path.

Example 43. The system according to any one of examples 1-42, further comprising:
an imaging device, positionable at the distal portion of the catheter; and
a data-processing system comprising means for carrying out the steps of Example 332.

Example 44. The system according to example 43, wherein the robot comprises a robotic control system that comprises the data-processing system, and that is configured to electronically operate the manipulator structure.

Example 45. The system according to any one of examples 1-44, further comprising:
an imaging device, positionable at the distal portion of the catheter; and
a data-processing system comprising means for carrying out the steps of Example 373.

Example 46. The system according to example 45, wherein the robot comprises a robotic control system that comprises the data-processing system, and that is configured to electronically operate the manipulator structure.

Example 47. The system according to any one of examples 1-46, wherein the advancement manipulator comprises a set of rollers.

Example 48. The system according to example 47, wherein the set of rollers is configured to rotate the catheter by rotating the tube.

Example 49. The system according to example 47, wherein the set of rollers is configured for single use.

Example 50. The system according to any one of examples 1-49, wherein the robot further comprises a robotic control system, configured to electronically control the manipulator structure.

Example 51. The system according to example 50, wherein the manipulator assembly is a first manipulator assembly, and the manipulator structure comprises a second manipulator assembly, the robotic control system configured to electronically coordinate control of the first manipulator assembly and the second manipulator assembly.

Example 52. The system according to example 51, further comprising a third manipulator assembly, and wherein the robotic control system is further configured to electronically coordinate control of the first manipulator assembly, the second manipulator assembly, and the third manipulator assembly.

Example 53. The system according to example 50, wherein:
the head comprises a stem, a first plunger, and a second plunger, and
the steering manipulator is configured to receive the head in a manner that operatively couples the steering manipulator to the steering region such that:
   the steering manipulator sliding the first plunger in a first direction along the stem bends the steering region, and
   the steering manipulator sliding the second plunger in a first direction along the stem straightens the steering region.

Example 54. The system according to example 53, wherein the robotic control system is configured to electronically receive information from the manipulator structure.

Example 55. The system according to example 54, wherein the robotic control system is configured to electronically control the manipulator structure responsively to the information.

Example 56. The system according to any one of examples 1-55, wherein the manipulator structure comprises a track that defines at least part of the advancement path.

Example 57. The system according to example 56, wherein the track comprises a rail.

Example 58. The system according to any one of examples 1-57, further comprising a mount configured to movably support the advancement manipulator.

Example 59. The system according to example 58, wherein the steering manipulator and the advancement manipulator are arranged horizontally with respect to each other.

Example 60. The system according to example 58, wherein the steering manipulator and the advancement manipulator are arranged vertically with respect to each other.

Example 61. The system according to example 58, wherein the steering manipulator and the advancement manipulator are arranged substantially perpendicular to a craniocaudal axis of a subject undergoing a procedure facilitated by the robot.

Example 62. The system according to any one of examples 1-61, further comprising a user interface enabled to facilitate, via the manipulator structure, advancement, steering, and rotation of the steering region.

Example 63. The system according to example 62, wherein the user interface comprises a hand-held controller.

Example 64. The system according to example 62, wherein the user interface comprises a display screen.

Example 65. The system according to any one of examples 1-64, further comprising a gate configured to secure the head in the steering manipulator.

Example 66. The system according to example 65, wherein the head is removable from the steering manipulator by opening the gate.

Example 67. The system according to example 66, wherein the catheter is configured to become limp responsively to the head being disengaged from the steering manipulator.

Example 68. The system according to any one of examples 1-67, wherein:
the head comprises:
   a stem,
   a first plunger, operatively coupled to the steering region via a first wire, and mounted on the stem to be slidable linearly along the stem, and
   a second plunger, operatively coupled to the steering region via a second wire, and mounted on the stem to be slidable linearly along the stem independently of the first plunger; and
the steering manipulator comprises:
   a first control unit, comprising:
      a first actuator,
      a first spring, and
      a first cradle, coupled to the first actuator via the first spring, and configured to receive the first plunger, and
   a second control unit, comprising:
      a second actuator,
      a second spring, and
      a second cradle, coupled to the second actuator via the second spring,
   and configured to receive the second plunger, and
the steering manipulator is configured to manipulate the steering region by:
   actuating the first actuator to, via the first spring, slide the first plunger linearly along the stem, and
   actuating the second actuator to, via the second spring, slide the second plunger linearly along the stem.

Example 69. The system according to example 68, wherein the catheter is configured such that linear sliding of the first plunger along the stem bends the steering region.

Example 70. The system according to example 68, wherein the catheter is configured such that linear sliding of the second plunger along the stem straightens the steering region.

Example 71. The system according to example 68, wherein the steering manipulator further comprises a third control unit, each of the first, second, and third control units being configured to pull a respective wire extending from the respective control unit to the steering region.

Example 72. The system according to example 68, wherein:
the steering manipulator comprises a housing, and
each of the first control unit and the second control unit comprises:
   an actuator encoder configured to provide an actuator output indicative of a linear position of the respective actuator with respect to the housing, and
   a cradle encoder configured to provide a cradle output indicative of a linear position of the respective cradle with respect to the housing.

Example 73. The system according to example 72, wherein the actuator encoder is fixedly coupled to the actuator.

Example 74. The system according to example 72, wherein the cradle encoder is fixedly coupled to the cradle.

Example 75. The system according to example 72, wherein the actuator output is indicative of a linear position of the actuator encoder with respect to the housing.

Example 76. The system according to example 72, wherein the cradle output is indicative of a linear position of the cradle encoder with respect to the housing.

Example 77. The system according to example 72, wherein the robot further comprises a robotic control system configured to use the cradle output and the actuator output to control a force applied to each spring by the respective actuator.

Example 78. The system according to example 72, wherein the actuator encoder comprises a first readhead paired with a first scale, and the cradle encoder comprises a second readhead paired with a second scale.

Example 79. The system according to example 78, wherein the first scale and the second scale are fixedly attached to the housing.

Example 80. The system according to example 78, wherein the first readhead is fixedly attached to the actuator and the second readhead is fixedly attached to the cradle.

Example 81. The system according to example 68, wherein, for each of the first control unit and the second control unit, when the corresponding plunger is disposed in the cradle, the control unit is configured such that a distance between the actuator and the cradle is dependent on tension in the corresponding wire.

Example 82. The system according to example 81, further comprising a robotic control system, configured to, for each of the first control unit and the second control unit, receive the actuator output and the cradle output, and determine a magnitude of tension in the corresponding wire responsively to the actuator output and the cradle output.

Example 83. The system according to example 82, wherein, for each of the first control unit and the second control unit, responsively to receiving the actuator output and the cradle output, the robotic control system is configured to calculate a distance between the actuator and the cradle, and to determine a magnitude of the tension responsively to the calculated distance.

Example 84. The system according to example 82, wherein the robotic control system is configured to balance tension on both wires.

Example 85. The system according to example 82, wherein the robotic control system is configured to adjust curvature of the steering region while maintaining stiffness on both wires.

Example 86. The system according to example 82, wherein, for each of the first control unit and the second control unit:
the spring has a predetermined spring constant, and
the robotic control system is configured to determine the magnitude of tension in the corresponding wire responsively to the actuator output, the cradle output, and the predetermined spring constant.

Example 87. The system according to example 82, wherein the robotic control system is configured to prevent the magnitude of the tension in the corresponding wire from rising above a predetermined level.

Example 88. The system according to example 82, wherein the robotic control system is configured to warn an operator about the magnitude of the tension in the corresponding wire rising above a predetermined level.

Example 89. The system according to example 78, wherein concurrent application of a balanced magnitude of tension to each wire at a point at which the steering region has a specific curvature is configured to stiffen the steering region in the specific curvature.

Example 90. The system according to example 68, wherein the steering manipulator further comprises:
a first motor configured to produce linear movement of the first actuator, and responsively, the first cradle and the first plunger; and
a second motor configured to produce linear movement of the second actuator, and responsively, the second cradle and the second plunger.

Example 91. The system according to example 90, wherein linear movement of the first plunger bends the steering region.

Example 92. The system according to example 90, wherein linear movement of the second plunger straightens the steering region.

Example 93. The system according to example 90, wherein rotation of the head rotates the steering region.

Example 94. The system according to example 90, wherein the steering manipulator further comprises a third motor.

Example 95. The system according to example 94, wherein the third motor is configured to rotate the head.

Example 96. The system according to any one of examples 1-95, further comprising a sensor configured to sense forward and rotational movement of the tube with respect to the advancement manipulator.

Example 97. The system according to example 96, wherein the sensor comprises:
a rider, the sensor being resiliently connected to the advancement manipulator in a manner that maintains contact between the rider and the tube such that the rider rolls responsively to movement of the tube, and
an optical reader mounted facing the rider, and configured to:
   detect rolling of the rider, and
   responsively to detecting the rolling, provide an output indicative of the movement.

Example 98. The system according to example 97, wherein rolling of the rider enables verification of movement of the tube by the optical reader in two degrees of motion.

Example 99. The system according to example 96, wherein the sensor is connected to the advancement manipulator in a manner that rolling of the rider follows movement of the tube.

Example 100. The system according to example 96, wherein the rider is spherical.

Example 101. The system according to example 96, wherein the sensor is disposed distally to the advancement manipulator.

Example 102. The system according to any one of examples 1-101, wherein:
the manipulator assembly is a first manipulator assembly,
the catheter is a first catheter, and
the system further comprises:
   a second catheter, and
   a second manipulator assembly, defining a second advancement path, such that the robot is configured to control the first catheter and the second catheter independently and in parallel with each other.

Example 103. The system according to example 102, wherein the system further comprises:
a third catheter, and
a third manipulator assembly, defining a third advancement path, such that the robot is configured to control the first catheter, the second catheter, and the third catheter independently and in parallel with each other.

Example 104. The system according to any one of examples 1-103, wherein the manipulator structure is configured such that the advancement manipulator is repositionable with respect to the steering manipulator.

Example 105. The system according to example 104, wherein the manipulator structure is configured such that the advancement manipulator is movable with respect to the steering manipulator in a manner that facilitates positioning of the advancement manipulator adjacent to an opening into a lumen of a subject.

Example 106. A system, comprising:
a catheter, comprising:
   a tube, having a steering region at a distal portion thereof;
   a first wire, and a second wire, each of the first wire and the second wire extending from the steering region proximally along the tube; and
   a head, comprising:
      a stem,
      a first plunger, operatively coupled to the steering region by being attached to the first wire, and mounted on the stem to be slidable linearly along the stem, and
      a second plunger, operatively coupled to the steering region by being attached to the second wire, and mounted on the stem to be slidable linearly along the stem independently of the first plunger; and
a steering manipulator configured to engage the head by receiving the head in a manner that operatively couples the steering manipulator to the first plunger and the second plunger in a manner that configures the steering manipulator to manipulate the steering region by controlling sliding of the first plunger and the second plunger linearly along the stem.

Example 107. The system according to example 106, wherein the catheter is sterilized.

Example 108. The system according to any one of examples 106-107, wherein the steering manipulator is configured to stiffen the steering region by facilitating balancing of tension between the first wire and the second wire.

Example 109. The system according to any one of examples 106-108, wherein the catheter is configured such that, while the steering region has a given curvature, balanced tension applied to the first wire and the second wire stiffens the steering region in the given curvature.

Example 110. The system according to any one of examples 106-109, wherein the first wire is operatively coupled to the steering region in a manner that causes deflection of the steering region in a first direction upon pulling of the first wire, and the second wire is operatively coupled to the steering region in a manner that causes deflection of the steering region in a second direction upon pulling of the second wire, the second direction being opposite to the first direction.

Example 111. The system according to any one of examples 106-110, wherein the steering manipulator is configured to control, via sliding of the first plunger and the second plunger linearly along the stem, (i) a curvature of the steering region, and (ii) a stiffness of the steering region.

Example 112. The system according to any one of examples 106-111, wherein the system is configured such that engaging the head with the steering manipulator enables the steering manipulator to rotate, bend, and straighten the steering region.

Example 113. The system according to any one of examples 106-112, wherein, for each of the first plunger and the second plunger, the plunger is mounted on the stem in a manner in which, while the head is not engaged by the steering manipulator, the plunger is slidable freely along at least part of the stem.

Example 114. The system according to any one of examples 106-113, wherein the first plunger is configured to tension the first wire to control a curvature of the steering region.

Example 115. The system according to any one of examples 106-114, wherein the second plunger is configured to tension the second wire to control a curvature of the steering region.

Example 116. The system according to any one of examples 106-115, wherein the steering manipulator comprises a motor enabled to rotate the catheter while the head is engaged with the steering manipulator.

Example 117. The system according to any one of examples 106-116, wherein the steering manipulator is configured to receive the head in a manner that enables the steering manipulator to, while remaining operatively coupled to the first plunger and the second plunger, rotate the steering region by rotating the head.

Example 118. The system according to any one of examples 106-117, wherein the system is configured such that engagement of the head by the steering manipulator enables measurement and calibration of tension on each of the first wire and the second wire of the catheter.

Example 119. The system according to any one of examples 106-118, wherein the catheter is configured such that, upon the head becoming released from the steering manipulator, the steering region responsively becomes limp.

Example 120. The system according to any one of examples 106-119, further comprising a robotic control system in electronic communication with the steering manipulator, and configured to identify the catheter upon the head being engaged by the steering manipulator.

Example 121. The system according to example 120, wherein the robotic control system is configured to, responsively to identifying the catheter, set one or more parameters for manipulation of the catheter.

Example 122. The system according to example 121, wherein the one or more parameters include allowable ranges of sliding of the first plunger and the second plunger linearly along the stem.

Example 123. The system according to example 121, wherein the one or more parameters include a maximum allowable force with which the steering manipulator may slide the first plunger linearly along the stem.

Example 124. The system according to example 121, wherein the one or more parameters include a maximum allowable force with which the steering manipulator may slide the second plunger linearly along the stem.

Example 125. The system according to any one of examples 106-124, wherein the steering manipulator comprises:
a first control unit, comprising:
   a first actuator,
   a first force sensor, and
   a first cradle, coupled to the first actuator via the first force sensor, and
   configured to receive the first plunger, and
a second control unit, comprising:
   a second actuator,
   a second force sensor, and
   a second cradle, coupled to the second actuator via the second force sensor, and
   configured to receive the second plunger,
the steering manipulator configured to manipulate the steering region by:
actuating the first actuator to, via the first force sensor, slide the first plunger linearly along the stem, and
actuating the second actuator to, via the second force sensor, slide the second plunger linearly along the stem.

Example 126. The system according to example 125, wherein the first actuator is a first linear actuator, and the second actuator is a second linear actuator.

Example 127. The system according to any one of examples 106-126, wherein the steering manipulator comprises:
a first control unit, comprising:
   a first actuator,
   a first spring, and
   a first cradle, coupled to the first actuator via the first spring, and configured to receive the first plunger, and
a second control unit, comprising:
   a second actuator,
   a second spring, and
   a second cradle, coupled to the second actuator via the second spring, and configured to receive the second plunger,
the steering manipulator configured to manipulate the steering region by:
   actuating the first actuator to, via the first spring, slide the first plunger linearly along the stem, and
   actuating the second actuator to, via the second spring, slide the second plunger linearly along the stem.

Example 128. The system according to example 127, wherein the first actuator is a first linear actuator, and the second actuator is a second linear actuator.

Example 129. The system according to example 127, wherein each of the first control unit and the second control unit is configured such that, when the plunger is disposed in the cradle, a linear distance between the actuator and the cradle is relative to tension in the corresponding wire.

Example 130. The system according to example 127, wherein, while the head is engaged by the steering manipulator, for each of the first plunger and the second plunger, the system is configured to maintain an axial position of the plunger with respect to the stem by automatically adjusting a force applied to the plunger by the actuator via the spring and the cradle.

Example 131. The system according to example 127, wherein the catheter is configured such that linear movement of the first plunger along the stem bends the steering region.

Example 132. The system according to example 127, wherein the catheter is configured such that linear movement of the second plunger along the stem straightens the steering region.

Example 133. The system according to example 127, wherein the steering manipulator comprises a motor enabled to rotate the catheter by rotating the head while the head is engaged with the steering manipulator.

Example 134. The system according to example 133, wherein, for each of the first plunger and the second plunger, while the head is engaged by the steering manipulator with the plunger disposed within the cradle, the steering manipulator is enabled to manipulate the steering region and rotate the head independently and concurrently.

Example 135. The system according to example 133, wherein each plunger is rotationally locked with respect to the stem.

Example 136. The system according to example 133, wherein each plunger has a circular outer cross-section, allowing it to slip rotationally within its cradle.

Example 137. The system according to example 127, wherein the steering manipulator further comprises:
a first motor configured to produce linear movement of the first actuator, and responsively, the first cradle and the first plunger; and
a second motor configured to produce linear movement of the second actuator, and responsively, the second cradle and the second plunger.

Example 138. The system according to example 137, wherein the steering manipulator further comprises a third motor.

Example 139. The system according to example 138, wherein the third motor is configured to rotate the head.

Example 140. The system according to example 127, wherein each of the first control unit and the second control unit comprises a cradle encoder configured to provide a cradle output indicative of a linear position of the cradle within the steering manipulator.

Example 141. The system according to example 140, wherein the cradle encoder is fixedly coupled to the cradle.

Example 142. The system according to example 140, wherein the cradle output is indicative of a linear position of the cradle encoder with respect to the steering manipulator.

Example 143. The system according to example 140, wherein each of the first control unit and the second control unit comprises an actuator encoder configured to provide an actuator output indicative of a linear position of the respective actuator within the steering manipulator.

Example 144. The system according to example 143, wherein the actuator encoder is fixedly coupled to the respective actuator.

Example 145. The system according to example 143, wherein the actuator output is indicative of a linear position of the actuator encoder with respect to the steering manipulator.

Example 146. The system according to example 140, wherein each of the first control unit and the second control unit comprises a force sensor configured to provide a force output indicative of strain on the spring resulting from actuation of the corresponding actuator.

Example 147. The system according to example 146, wherein the force sensor comprises a strain gauge.

Example 148. The system according to example 146, wherein the force sensor is disposed between the cradle and the actuator.

Example 149. The system according to example 140, wherein each of the first control unit and the second control unit comprises a force sensor configured to provide a force output indicative of force exerted on the cradle by the corresponding actuator via the corresponding spring.

Example 150. The system according to example 140, wherein:
each of the first control unit and the second control unit comprises another encoder, configured to provide another output, and
the system further comprises a robotic control system configured to, for each of the first control unit and the second control unit:
receive the cradle output and the other output, and
responsively to the cradle output and the other output, determine a tension magnitude in the corresponding wire.

Example 151. The system according to example 150, wherein, for each of the first control unit and the second control unit:
the other encoder is an actuator encoder,
the other output is an actuator output indicative of a linear position of the actuator within the steering manipulator, and
the actuator encoder is configured to provide the actuator output.

Example 152. The system according to example 150, wherein, for each of the first control unit and the second control unit:
the other output is indicative of a linear position of the actuator with respect to the cradle, and
the other encoder is configured to provide the other output that is indicative of the linear position of the actuator with respect to the cradle.

Example 153. The system according to example 150, wherein, for each of the first control unit and the second control unit, responsively to receiving the cradle output and the other output, the control system is configured to calculate a distance between the actuator and the cradle, and to determine the tension magnitude in the corresponding wire responsively to the calculated distance.

Example 154. The system according to example 150, wherein, for each of the first control unit and the second control unit:
the spring has a predetermined spring constant,
the other output is the actuator output, and
the control system is configured to determine the tension magnitude in the corresponding wire responsively to the actuator output, the cradle output, and the predetermined spring constant.

Example 155. The system according to example 150, wherein, for each of the first control unit and the second control unit:
the other output is a force sensor output, and
the robotic control system is configured to determine the tension magnitude in the corresponding wire responsively to the force sensor output.

Example 156. The system according to example 150, wherein the robotic control system is configured to disallow the steering manipulator from increasing the tension magnitude in the corresponding wire to above a predetermined level.

Example 157. The system according to example 150, wherein the robotic control system is configured to warn an operator upon the tension magnitude in the corresponding wire rising above a predetermined level.

Example 158. The system according to example 150, wherein the robotic control system is configured to stiffen the steering region by balancing the tension magnitude of the first wire with the tension magnitude of the second wire.

Example 159. The system according to example 150, wherein the catheter is configured such that, while the steering region has a given curvature, balanced tension applied to each wire stiffens the steering region in the given curvature.

Example 160. The system according to example 140, wherein the actuator encoder comprises a first readhead paired with a first scale, and the cradle encoder comprises a second readhead paired with a second scale.

Example 161. The system according to example 160, wherein the first scale is fixedly attached to the steering manipulator.

Example 162. The system according to example 160, wherein the second scale is fixedly attached to the steering manipulator.

Example 163. The system according to example 160, wherein the first readhead is fixedly attached to the corresponding actuator.

Example 164. The system according to example 160, wherein the second readhead is fixedly attached to the cradle.

Example 165. The system according to example 127, wherein each of the first control unit and the second control unit comprises an actuator encoder configured to provide an actuator output indicative of a linear position of the actuator within the steering manipulator.

Example 166. The system according to example 165, wherein the actuator encoder is fixedly coupled to the actuator.

Example 167. The system according to example 165, wherein the actuator output is indicative of a linear position of the actuator encoder with respect to the steering manipulator.

Example 168. The system according to example 127, wherein each of the first control unit and the second control unit comprises a force sensor configured to provide a force output indicative of strain on the spring resulting from actuation of the corresponding actuator.

Example 169. The system according to example 168, wherein the force sensor comprises a strain gauge.

Example 170. The system according to example 168, wherein the force sensor is disposed between the cradle and the actuator.

Example 171. The system according to example 127, wherein each of the first control unit and the second control unit comprises a force sensor configured to provide a force output indicative of force exerted on the cradle by the corresponding actuator via the corresponding spring.

Example 172. The system according to example 127, wherein each of the first control unit and the second control unit is configured to provide an output indicative of a linear distance between the cradle and the actuator.

Example 173. The system according to example 172, wherein, for each of the first control unit and the second control unit, the control unit comprises a movable linear scale, fixed with respect to the actuator, and the control unit is configured to provide the output responsively to a linear position of the cradle with respect to the movable linear scale.

Example 174. The system according to example 172, wherein, for each of the first control unit and the second control unit, the control unit comprises a movable linear scale, fixed with respect to the cradle, and the control unit is configured to provide the output responsively to a linear position of the actuator with respect to the movable linear scale.

Example 175. The system according to example 127, wherein each of the first control unit and the second control unit comprises a first encoder and a second encoder.

Example 176. The system according to example 175, wherein, for each of the first control unit and the second control unit:
the first encoder is configured to provide an output indicative of a position of the cradle within the steering manipulator, and
the second encoder is configured to provide an output indicative of a linear distance between the cradle and the actuator.

Example 177. The system according to example 175, wherein, for each of the first control unit and the second control unit:
the first encoder is configured to provide an output indicative of a position of the actuator within the steering manipulator, and
the second encoder is configured to provide an output indicative of a linear distance between the cradle and the actuator.

Example 178. The system according to example 175, wherein, for each of the first control unit and the second control unit:
the first encoder is configured to provide an output indicative of a position of the cradle within the steering manipulator, and
the second encoder is configured to provide an output indicative of a position of the actuator within the steering manipulator.

Example 179. The system according to example 175, wherein:
the system further comprises a control system,
each of the first encoder and the second encoder is configured to provide a respective output, and
the control system is configured to receive the respective output of each of the first encoder and the second encoder.

Example 180. The system according to any one of examples 106-179, wherein the first wire is operatively coupled to the steering region in a pulley arrangement configured to increase a mechanical advantage of the first wire on the steering region.

Example 181. The system according to example 180, wherein the second wire is operatively coupled to the steering region in a second pulley arrangement configured to increase a mechanical advantage of the second wire on the steering region.

Example 182. The system according to any one of examples 106-181, further comprising a track on which the steering manipulator is slidably mounted.

Example 183. The system according to example 182, wherein the slidable mounting of the steering manipulator on the track enables distal advancement and proximal retreat of the steering manipulator.

Example 184. The system according to example 183, wherein the steering manipulator is biased to retreat proximally along the track.

Example 185. The system according to example 184, wherein the steering manipulator is biased to retreat proximally along the track by the track sloping downward proximally.

Example 186. The system according to example 184, wherein the steering manipulator is biased to retreat proximally along the track by a pulley attached to the slidable mounting.

Example 187. The system according to example 184, wherein the steering manipulator is biased to retreat proximally along the track by a spring.

Example 188. The system according to example 184, wherein the steering manipulator is biased to retreat proximally along the track by gravitational pull.

Example 189. The system according to example 188, wherein the system further comprises a counterweight, coupled to the steering manipulator in a manner that provides the gravitational pull.

Example 190. The system according to example 188, wherein the track is sloped in a manner that provides the gravitational pull.

Example 191. The system according to any one of examples 106-190, wherein the first plunger and the second plunger and the stem are complementarily shaped to rotationally lock the first plunger and the second plunger to the stem.

Example 192. The system according to example 191, wherein the complementary shapes define a keyed joint.

Example 193. The system according to example 191, wherein the stem has a noncircular outer cross-section, and each of the first plunger and the second plunger has a complementary noncircular inner cross-section, thereby rotationally locking the first plunger and the second plunger to the stem.

Example 194. The system according to example 191, wherein the steering manipulator comprises:
a first cradle, configured to, upon the steering manipulator receiving the head, cradle the first plunger in a manner that operatively couples the steering manipulator to the first plunger while allowing the first plunger to slip rotationally within the first cradle, and
a second cradle, configured to, upon the steering manipulator receiving the head, cradle the second plunger in a manner that operatively couples the steering manipulator to the second plunger while allowing the second plunger to slip rotationally within the second cradle.

Example 195. The system according to example 194, wherein the steering manipulator is configured to rotate the head and to cause the rotational slipping of the first plunger and the second plunger.

Example 196. The system according to any one of examples 106-195, wherein:
the catheter is a first catheter,
the steering manipulator is a first steering manipulator, and
the system further comprises a second catheter and a second steering manipulator configured to engage a head of the second catheter in a manner that configures the steering manipulator to manipulate a steering region of the second catheter.

Example 197. The system according to example 196, wherein the system further comprises a robotic control system, electronically couplable to both the first steering manipulator and the second steering manipulator in a manner that enables the robotic control system to coordinate control of both the first catheter and the second catheter.

Example 198. The system according to example 197, wherein:
the system further comprises a third catheter, and a third steering manipulator, and
the robotic control system is electronically couplable to the first steering manipulator, the second steering manipulator, and the third steering manipulator in a manner that enables the robotic control system to coordinate control of the first catheter, the second catheter, and the third catheter.

Example 199. The system according to any one of examples 106-198, further comprising a gate configured to maintain engagement of the head by the steering manipulator by securing the head within the steering manipulator.

Example 200. The system according to example 199, wherein the head defines a circular surface, and the gate comprises a roller configured to ride on the circular surface, facilitating rotation of the catheter while the gate maintains the head secured within the steering manipulator.

Example 201. Apparatus, comprising:
a catheter, comprising:
   a tube, having a steering region at a distal portion thereof;
   a first wire, and a second wire, each of the first wire and the second wire extending from the steering region proximally along the tube; and
   a head, comprising:
      a stem,
      a first plunger, mounted on the stem, and operatively coupled to the steering region via the first wire, such that linear sliding of the first plunger along the stem deflects the steering region in a first direction by pulling on the first wire, and
      a second plunger, mounted on the stem, and operatively coupled to the steering region via the second wire, such that linear sliding of the second plunger along the stem deflects the steering region in a second direction by pulling on the second wire.

Example 202. The apparatus according to example 201, wherein the catheter is sterilized.

Example 203. The apparatus according to any one of examples 201-202, wherein the head is absent of means to maintain a linear position, along the stem, of either of the first plunger and the second plunger.

Example 204. The apparatus according to any one of examples 201-203, wherein the head further comprises a head-gearwheel, fixed to the stem.

Example 205. The apparatus according to any one of examples 201-204, wherein the first plunger and the second plunger are linearly slidable along the stem independently of each other.

Example 206. The apparatus according to any one of examples 201-205, wherein the stem extends through the first plunger and the second plunger.

Example 207. The apparatus according to any one of examples 201-206, further comprising a holder, removably mounted on the head in a manner that inhibits linear sliding of the first plunger along the stem.

Example 208. The apparatus according to example 207, wherein the holder is shaped to define a handle that facilitates handling of the head by a human operator.

Example 209. The apparatus according to example 207, wherein the manner in which the holder is mounted on the head inhibits linear sliding of the second plunger along the stem.

Example 210. The apparatus according to example 209, wherein the manner in which the holder is mounted on the head retains the first plunger at a first-plunger linear position along the stem, and retains the second plunger at a second-plunger linear position along the stem.

Example 211. The apparatus according to any one of examples 201-210, further comprising an electronically-controlled steering manipulator, the head being loadable into the steering manipulator in a manner that operatively couples the steering manipulator to the steering region such that the steering region is adjustable via sliding, by the steering manipulator, of the first plunger and the second plunger linearly along the stem.

Example 212. The apparatus according to example 211, wherein:
the steering manipulator comprises a motor,
the head further comprises a head-gearwheel, fixed to the stem, and
the manner in which the head is loadable into the steering manipulator operatively couples the head-gearwheel to the motor such that the catheter is rotatable by the motor driving the head-gearwheel.

Example 213. The apparatus according to example 212, wherein:
the motor is operatively coupled to a drive axle, and
the apparatus further comprises a single-use gearwheel, configured to be temporarily mounted on the drive axle such that loading the head into the steering manipulator operatively couples the motor to the head-gearwheel via the drive axle and the single-use gearwheel, the single-use gearwheel being discardable with the catheter after use.

Example 214. The apparatus according to example 211, wherein the steering manipulator defines a first push-face with which the steering manipulator is configured to push the first plunger linearly along the stem, and a second push-face with which the steering manipulator is configured to push the second plunger linearly along the stem.

Example 215. The apparatus according to example 214, wherein the steering manipulator is configured to push the first plunger proximally along the stem by pushing the first push-face proximally against the first plunger.

Example 216. The apparatus according to example 215, wherein the steering manipulator is configured to push the second plunger proximally along the stem by pushing the second push-face proximally against the second plunger.

Example 217. The apparatus according to example 215, further comprising a holder, removably mounted on the head in a manner that maintains the first plunger at a first-plunger linear position along the stem, and maintains the second plunger at a second-plunger linear position along the stem, such that, upon loading of the head into the steering manipulator, the first plunger is positioned at the first push-face, and the second plunger is positioned at the second push-face.

Example 218. The apparatus according to example 217, wherein the holder is shaped to define a handle that facilitates loading of the head into the steering manipulator by a human operator.

Example 219. The apparatus according to example 217, wherein the holder is removably mounted on the head by snap-fit.

Example 220. The apparatus according to any one of examples 201-219, wherein the catheter comprises a roller assembly, mounted on the tube, and comprising a roller configured to feed the tube through the roller assembly.

Example 221. The apparatus according to example 220, wherein the apparatus comprises a sterile package containing the catheter.

Example 222. The apparatus according to example 221, wherein the head further comprises a head-gearwheel, fixed to the stem, and wherein the sterile package further contains at least one manipulator-gearwheel that is complementary to the head-gearwheel.

Example 223. The apparatus according to example 220, wherein the roller is a roller of a set of rollers, the set of rollers being configured to feed the tube through the roller assembly.

Example 224. The apparatus according to example 220, wherein the roller is configured to be pressed into engagement with the tube.

Example 225. The apparatus according to example 224, wherein the roller assembly has a rest state in which the roller is disengaged from the tube.

Example 226. The apparatus according to example 220, further comprising an electronically-controlled advancement manipulator, wherein the roller assembly is loadable onto the advancement manipulator in a manner that configures the advancement manipulator to feed the tube through the roller assembly by rotating the roller.

Example 227. The apparatus according to example 226, wherein the advancement manipulator is configured to press the roller into engagement with the tube.

Example 228. The apparatus according to example 227, wherein the advancement manipulator is configured to press the roller into engagement with the tube upon loading of the roller assembly onto the advancement manipulator.

Example 229. The apparatus according to example 226, wherein the advancement manipulator comprises an axle, and the roller assembly is loadable onto the advancement manipulator in a manner that mounts the roller on the axle such that the advancement manipulator becomes configured to feed the tube through the roller assembly by rotating the roller via rotating the axle.

Example 230. The apparatus according to example 227, wherein the roller assembly is configured such that the mounting of the roller onto the axle presses the roller into engagement with the tube.

Example 231. Apparatus, comprising:
a catheter, comprising:
   a tube, having a steering region at a distal portion thereof;
   a head, at a proximal region of the tube, and operatively coupled to the steering region such that operation of the head controls bending of the steering region; and
   a feeder, mounted on the tube, and configured to feed the tube through the feeder.

Example 232. The apparatus according to example 231, wherein the catheter is sterilized.

Example 233. The apparatus according to any one of examples 231-232, wherein the apparatus comprises a sterile package containing the catheter.

Example 234. The apparatus according to any one of examples 231-233, wherein the apparatus is for use with an advancement manipulator, the feeder being loadable onto the advancement manipulator in a manner that configures the advancement manipulator to drive the feeder to feed the tube through the feeder.

Example 235. The apparatus according to any one of examples 231-234, wherein the feeder is passive, and is configured to be driven to feed the tube through the feeder.

Example 236. The apparatus according to any one of examples 231-235, wherein the feeder comprises a roller configured to feed the tube through the feeder.

Example 237. The apparatus according to example 236, wherein the feeder has a rest state in which the feeder is mounted on the tube and the roller is disengaged from the tube.

Example 238. The apparatus according to example 237, wherein the roller is configured to be pressed into engagement with the tube.

Example 239. The apparatus according to example 236, wherein the roller is a feed roller, and wherein the feeder further comprises a rider roller that has a rider-roller axis, and that is mounted in the feeder such that feeding of the tube through the feeder by the feed roller rotates the rider roller about the rider-roller axis.

Example 240. The apparatus according to example 239, wherein the feeder is configured to feed the tube through the feeder via rotation of the feed roller about a feed-roller axis, and the feed roller is mounted in the feeder such that rotation of the tube within the feeder translates the feed roller along the feed-roller axis.

Example 241. The apparatus according to example 240, wherein:
the feed roller is a first feed roller, the feed-roller axis being a first feed-roller axis,
the feeder comprises a second feed roller, complementary to the first feed roller,
the feeder is configured to feed the tube through the feeder via concurrent rotation (i) of the first feed roller in a first rotational direction about the first feed-roller axis, and (ii) of the second feed roller in a second rotational direction about a second feed-roller axis, the second rotational direction being opposite to the first rotational direction, and
the first feed roller and the second feed roller are mounted in the feeder such that rotation of the tube within the feeder concurrently translates (i) the first feed roller in a first axial direction along the first feed-roller axis, and (ii) the second feed roller in a second axial direction along the second feed-roller axis, the second axial direction being opposite to the first axial direction.

Example 242. The apparatus according to example 239, wherein the rider roller is mounted in the feeder such that rotation of the tube within the feeder translates the rider roller along the rider-roller axis.

Example 243. The apparatus according to example 242, wherein:
the rider roller is a first rider roller, the rider-roller axis being a first rider-roller axis,
the feeder comprises a second rider roller, complementary to the first rider roller,
the first rider roller and the rider roller are mounted in the feeder such that:
   feeding of the tube through the feeder by the feed roller concurrently rotates (i) the first rider roller in a first rotational direction about the first rider-roller axis, and (ii) the second rider roller in a second rotational direction about a second rider-roller axis, the second rotational direction being opposite to the first rotational direction, and
   rotation of the tube within the feeder concurrently translates (i) the first rider roller in a first axial direction along the first rider-roller axis, and (ii) the second rider roller in a second axial direction along the second rider-roller axis, the second axial direction being opposite to the first axial direction.

Example 244. The apparatus according to example 242, further comprising an electronically-controlled advancement manipulator, the feeder being loadable onto the advancement manipulator in a manner that configures the advancement manipulator to feed the tube through the feeder by rotating the feed roller.

Example 245. The apparatus according to example 244, wherein the advancement manipulator comprises a sensor, and wherein, when the feeder is loaded onto the advancement manipulator, the advancement manipulator is configured to:
detect the rotation of the rider roller about the rider-roller axis,
responsively to the detected rotation, provide an advancement output indicative of advancement of the tube through the feeder,
detect the translation of the rider roller along the rider-roller axis, and
responsively to the detected translation, provide a rotation output indicative of rotation of the tube within the feeder.

Example 246. The apparatus according to example 244, wherein:
the advancement manipulator comprises:
   a motorized feed axle; and
   a passive rider axle, and
the feeder is configured such that loading the feeder onto the advancement manipulator:
   loads the feed roller onto the feed axle such that the feed roller becomes rotationally locked to the feed axle, but axially slidable along the feed axle, and
   loads the rider roller onto the rider axle such that the rider roller becomes rotationally and axially locked to the rider axle.

Example 247. The apparatus according to example 246, wherein the feed axle and the rider axle are spring-loaded such that, when the feeder is loaded onto the advancement manipulator, the feed axle presses the feed roller into engagement with the tube and the rider axle presses the rider roller into engagement with the tube.

Example 248. Apparatus for use with an elongate member, the apparatus comprising an advancement unit that comprises:
a feed axle that lies on a feed-axle axis;
a motor, operatively coupled to the feed axle such that operation of the motor rotates the feed axle about the feed-axle axis;
a rider axle that lies on a rider-axle axis; and
one or more sensors, operatively coupled to the rider axle in a manner that detects:
   rotation of the rider axle about the rider-axle axis, and
   translation of the rider axle along the rider-axle axis,
      the advancement unit being configured to receive the elongate member such that:
operation of the motor feeds the elongate member through the advancement unit via rotation of the feed axle about the feed-axle axis in a manner in which the elongate member rotates the rider axle about the rider-axle axis, and
rotation of the elongate member within the advancement unit translates the rider axle along the rider-axle axis.

Example 249. The apparatus according to example 248, further comprising a feed roller mounted on the feed axle, and configured to engage the elongate member such that operation of the motor feeds the elongate member through the advancement unit by rotating the feed roller about the feed-axle axis.

Example 250. The apparatus according to example 249, wherein the advancement unit is configured to receive the elongate member such that rotation of the elongate member within the advancement unit slides the feed roller along the feed axle.

Example 251. The apparatus according to any one of examples 248-250, further comprising a rider roller mounted on the rider axle, and configured to engage the elongate member such that feeding of the elongate member through the advancement unit rotates the rider axle about the rider-axle axis by rotating the rider roller about the rider-axle axis.

Example 252. The apparatus according to example 251, wherein the advancement unit is configured to receive the elongate member such that rotation of the elongate member within the advancement unit translates the rider axle along the rider-axle axis by translating the rider roller along the rider-axle axis.

Example 253. Apparatus comprising a catheter for use in operative procedures, the catheter comprising:
a tube having a steering region at a distal portion thereof,
a set of wires, extending proximally along the tube from the steering region, and
a head, attached to a proximal portion of the tube, and operatively coupled to the steering region via the set of wires such that:
curvature of the steering region is controllable by manipulating the head to apply tension to the set of wires, and
the head is configured to not maintain the tension in the wires of the set in an absence of an exogenous force applied to the head.

Example 254. The apparatus according to example 253, wherein the catheter is sterilized.

Example 255. The apparatus according to any one of examples 253-254, wherein the wires of the set are tensionable independently of each other.

Example 256. The apparatus according to any one of examples 253-255, wherein the set of wires comprises exactly three wires distributed circumferentially around the tube and extending proximally along the tube.

Example 257. The apparatus according to any one of examples 253-256, wherein the set of wires comprises exactly two wires extending, opposite each other, proximally along the tube.

Example 258. The apparatus according to any one of examples 253-257, wherein the catheter is a single-use catheter.

Example 259. The apparatus according to any one of examples 253-258, wherein the catheter is configured such that increasing tension on all of the wires of the set stiffens the steering region.

Example 260. The apparatus according to example 259, wherein the steering region of the tube comprises intercalating vertebrae.

Example 261. The apparatus according to example 260, wherein the stiffening of the steering region is facilitated by the increasing of the tension on all of the wires axially compressing the intercalating vertebrae.

Example 262. The apparatus according to example 260, wherein the vertebrae are configured such that, when the steering region is stiffened, the vertebrae slip against each other in a single direction.

Example 263. The apparatus according to example 260, wherein the vertebrae are configured such that, when the steering region is stiffened, the vertebrae slip against each other in any direction.

Example 264. The apparatus according to any one of examples 253-263, wherein:
the set of wires comprises:
   a bending wire, tensioning of the bending wire bending the steering region, and
   a straightening wire, tensioning of the bending wire straightening the steering region, and
the head comprises:
   a stem, fixed to the proximal portion of the tube,
   a bending plunger attached to the bending wire, and slidable along the stem in a manner that applies tension to the bending wire, and
   a straightening plunger attached to the straightening wire, and slidable along the stem in a manner that applies tension to the straightening wire.

Example 265. The apparatus according to example 264, wherein, within the steering region, the bending wire is arranged in a force-multiplication arrangement that provides the bending wire with a mechanical advantage.

Example 266. The apparatus according to example 265, wherein, within the steering region, the mechanical advantage of the bending wire is greater than a mechanical advantage of the straightening wire.

Example 267. The apparatus according to example 265, wherein, within the steering region, the straightening wire is not arranged in a force-multiplication arrangement.

Example 268. The apparatus according to example 265, wherein the mechanical advantage is at least two.

Example 269. The apparatus according to example 265, wherein the mechanical advantage is at least three.

Example 270. The apparatus according to example 265, wherein the force-multiplication arrangement is a pulley system.

Example 271. The apparatus according to any one of examples 253-270, further comprising a steering manipulator, reversibly engageable with the head, and configured to apply and maintain the exogenous force on the head.

Example 272. The apparatus according to example 271, further comprising a control system configured to electronically control the steering manipulator to apply and maintain the exogenous force on the head.

Example 273. Apparatus, comprising a catheter for use in operative procedures, the catheter comprising:
a tube having a steering region at a distal portion thereof,
a wire, extending from the steering region proximally along the tube,
a head, attached to a proximal portion of the tube, and comprising:
   a stem, and
   a plunger, slidably coupled to the stem, and connected to the wire such that:
   application of a sliding force to the plunger slides the plunger in a first direction along the stem in a manner that increases a curvature of the steering region by pulling on the wire, and
   releasing the sliding force allows the wire to responsively pull the plunger in a reverse direction along the stem.

Example 274. The apparatus according to example 273, wherein the catheter is sterilized.

Example 275. The apparatus according to any one of examples 273-274, wherein the stem is elongate and has a linear axis.

Example 276. The apparatus according to any one of examples 273-275, wherein:
the plunger is a first plunger, and the head further comprises a second plunger slidably coupled to the stem; and
the wire is a first wire, and
the catheter further comprises a second wire, extending from the steering region proximally along the tube, and connected to the second plunger, such that application of a sliding force to the second plunger slides the second plunger in the first direction along the stem in a manner that decreases a curvature of the steering region by applying tension to the second wire.

Example 277. The apparatus according to example 276, wherein the first plunger is disposed on the stem axially between the second plunger and the tube.

Example 278. The apparatus according to example 276, wherein the steering region of the tube is stiffenable by balanced tensioning of the first wire and the second wire.

Example 279. The apparatus according to example 276, further comprising a steering manipulator, reversibly engageable with the head, and configured to tension the first wire and the second wire independently of each other.

Example 280. Apparatus, comprising a catheter for use in operative procedures, the catheter comprising:
a flexible tube having a proximal region, an intermediate region, and a steering region distal to the intermediate region;
a first wire and a second wire, each of the first wire and the second wire extending from the steering region proximally along the tube, and
a head, attached to a proximal portion of the tube, to the first wire, and to the second wire, and configured:
   to facilitate bending of the steering region by pulling on the first wire, and
   to facilitate stiffening of the steering region via application of tension to the first wire and the second wire concurrently.

Example 281. The apparatus according to example 280, wherein the catheter is sterilized.

Example 282. The apparatus according to any one of examples 280-281, wherein the intermediate region is more flexible than the steering region.

Example 283. The apparatus according to any one of examples 280-282, wherein the steering region comprises a series of vertebrae having a predetermined compressive strength.

Example 284. The apparatus according to example 283, wherein the vertebrae are configured to lock in a fixed curvature responsively to the application of the tension to the first wire and the second wire concurrently.

Example 285. The apparatus according to any one of examples 280-284, wherein the head comprises:
a stem,
a first plunger, connected to the first wire, and slidably coupled to the stem, and
a second plunger, connected to the second wire, and slidably coupled to the stem independently of the first plunger.

Example 286. The apparatus according to example 285, wherein the head is configured such that, for each of the first plunger and the second plunger, application of a linear force to the plunger pulls on the corresponding wire.

Example 287. The apparatus according to example 286, further comprising a steering manipulator, reversibly engageable with the head, and configured to apply linear force to the first plunger and to apply linear force to the second plunger.

Example 288. The apparatus according to example 287, further comprising a control system configured to electronically control the steering manipulator to apply linear force to the first plunger and to apply linear force to the second plunger.

Example 289. The apparatus according to example 288, wherein the control system is configured to electronically control the steering manipulator to stiffen the steering region by applying tension to the first wire and the second wire concurrently.

Example 290. The apparatus according to any one of examples 280-289, wherein, within the steering region, the first wire is arranged to have a greater mechanical advantage than in the intermediate region.

Example 291. The apparatus according to example 290, wherein, within the steering region, the greater mechanical advantage is provided by the first wire being arranged in a pulley arrangement.

Example 292. The apparatus according to example 290, wherein, within the steering region, the second wire is arranged to have a greater mechanical advantage than in the intermediate region.

Example 293. A method, comprising:
advancing a steering region at a distal portion of a tube of a catheter into a real or simulated subject, the catheter including a first wire and a second wire extending from the steering region proximally along the tube;
adjusting a curvature of the steering region by pulling the first wire proximally relative to the tube and to the second wire; and
subsequently, stabilizing the curvature by balancing tension between the first wire and the second wire.

Example 294. The method according to example 293, wherein the steps of adjusting the curvature of the steering region and advancing the steering region are performed concurrently.

Example 295. The method according to any one of examples 293-294, wherein the steps of adjusting the curvature of the steering region and stabilizing the curvature of the steering region are performed concurrently.

Example 296. The method according to any one of examples 293-295, wherein adjusting the curvature comprises pulling the first wire.

Example 297. The method according to example 296, wherein stabilizing the curvature comprises pulling the second wire while maintaining the steering region in a desired curvature.

Example 298. The method according to any one of examples 293-297, wherein advancing the steering region into the subject comprises transluminally advancing the steering region into the subject.

Example 299. The method according to example 298, wherein transluminally advancing the steering region into the subject comprises transbronchially advancing the steering region into the subject.

Example 300. The method according to any one of examples 293-299, further comprising sliding a medical tool within a lumen of the tube while the curvature of the steering region remains stabilized.

Example 301. The method according to example 300, wherein sliding the medical tool within the lumen comprises advancing the medical tool distally through the lumen.

Example 302. The method according to example 300, wherein sliding the medical tool within the lumen comprises retracting the medical tool proximally through the lumen.

Example 303. The method according to example 300, wherein sliding the medical tool within the lumen comprises advancing a camera through the lumen.

Example 304. The method according to example 300, wherein sliding the medical tool within the lumen comprises advancing a needle through the lumen.

Example 305. The method according to example 300, wherein sliding the medical tool within the lumen comprises advancing an ultrasound probe through the lumen.

Example 306. The method according to any one of examples 293-305, wherein advancing the steering region into the subject is robotically performed by an advancement manipulator.

Example 307. The method according to example 306, wherein adjusting the curvature of the steering region and stabilizing the curvature are robotically performed by a steering manipulator.

Example 308. The method according to any one of examples 293-307, wherein the catheter is a first catheter, and the method further comprises:
advancing a steering region at a distal portion of a tube of a second catheter into the subject, the second catheter including a first wire and a second wire extending from the steering region proximally along the tube;
adjusting a curvature of the steering region of the second catheter by pulling the first wire of the second catheter proximally relative to the tube of the second catheter and to the second wire of the second catheter; and subsequently, stabilizing the curvature of the steering region of the second catheter by balancing tension between the first wire and the second wire of the second catheter.

Example 309. The method according to example 308, wherein advancing the steering region of the second catheter into the subject comprises advancing the steering region of the second catheter into the subject while the steering region of the first catheter remains within the subject.

Example 310. The method according to example 308, wherein adjusting the curvature of the steering region of the second catheter comprises adjusting the curvature of the steering region of the second catheter while the steering region of the first catheter remains within the subject.

Example 311. A method, comprising:
advancing a steering region at a distal portion of a tube of a catheter into a real or simulated subject, the catheter including a first wire and a second wire extending from the steering region proximally along the tube;
concurrently tensioning the first wire and the second wire; and
adjusting a curvature of the steering region within the subject.

Example 312. The method according to example 311, wherein adjusting a curvature of the steering region comprises adjusting a curvature of the steering region while maintaining the concurrent tensioning on the first wire and the second wire.

Example 313. The method according to any one of examples 311-312, wherein advancing the steering region into the subject comprises transbronchially advancing the steering region into the subject.

Example 314. The method according to any one of examples 311-313, wherein the catheter is a first catheter, and the method further comprises:
advancing a steering region at a distal portion of a tube of a second catheter into the subject, the second catheter including a first wire and a second wire extending from the steering region proximally along the tube;
concurrently tensioning the first wire and the second wire of the second catheter; and
adjusting a curvature of the steering region of the second catheter within the subject.

Example 315. The method according to example 314, wherein advancing the steering region of the second catheter into the subject comprises advancing the steering region of the second catheter into the subject while the steering region of the first catheter remains within the subject.

Example 316. The method according to example 314, wherein adjusting the curvature of the steering region of the second catheter comprises adjusting the curvature of the steering region of the second catheter while the steering region of the first catheter remains within the subject.

Example 317. The method according to any one of examples 311-316, wherein:
the first wire is arranged within the steering region to define a force-multiplication arrangement, and
adjusting the curvature of the steering region comprises adjusting the curvature of the steering region by applying a tensioning force to a proximal end of the first wire such that the tensioning force is multiplied, within the steering region, by the force-multiplication arrangement.

Example 318. A system, comprising:
a tube feeder, comprising a set of rollers, and configured to feed a tube therethrough, and
a sensor comprising a rider configured to roll passively in response to movement of the tube, the sensor configured:
to detect passive rolling of the rider, and
responsively to detecting the rolling, to provide an output indicative of the movement.

Example 319. The system according to example 318, wherein the tube feeder is sterilized.

Example 320. The system according to any one of examples 318-319, wherein at least the rider of the sensor is sterilized.

Example 321. The system according to any one of examples 318-320, wherein rolling of the rider enables verification of movement of the tube by the sensor in two degrees of motion.

Example 322. The system according to example 318, wherein the rider is spherical.

Example 323. The system according to example 318, wherein the rider is cylindrical.

Example 324. The system according to example 318, wherein the sensor comprises an optical reader.

Example 325. The system according to example 318, wherein the sensor comprises an electromechanical encoder.

Example 326. The system according to example 318, wherein the sensor is disposed downstream of the tube feeder.

Example 327. Apparatus, comprising a steering manipulator for use with a catheter comprising a head and a steering region, the steering manipulator comprising:
a housing;
a first control unit, comprising:
   a first actuator,
   a first spring, and
   a first cradle, coupled to the first actuator via the first spring, and configured to receive a first plunger of the head of the catheter,
   a first actuator encoder configured to provide a first actuator output indicative of a linear position of the first actuator with respect to the housing, and
   a first cradle encoder configured to provide a cradle output indicative of a linear position of the cradle with respect to the housing; and
a second control unit, comprising:
   a second actuator,
   a second spring, and
   a second cradle, coupled to the second actuator via the second spring, and configured to receive a second plunger of the head of the catheter, and
   a second actuator encoder configured to provide a second actuator output indicative of a linear position of the second actuator with respect to the housing, and
   a second cradle encoder configured to provide a second cradle output indicative of a linear position of the second cradle with respect to the housing;
      wherein the steering manipulator is configured to receive the head of the catheter in a manner that operatively couples the steering manipulator to the head, enabling the steering manipulator to manipulate the steering region by:
actuating the first actuator to, via the first spring, slide the first plunger linearly along a stem of the head of the catheter, and
actuating the second actuator to, via the second spring, slide the second plunger linearly along the stem.

Example 328. The apparatus according to example 327, wherein linear movement of the first plunger along the stem is configured to bend the steering region.

Example 329. The apparatus according to any one of examples 327-328, wherein linear movement of the second plunger along the stem is configured to straighten the steering region.

Example 330. The apparatus according to any one of examples 327-329, wherein the steering manipulator further comprises a third motor.

Example 331. The apparatus according to example 330, wherein the third motor is configured to rotate the head.

Example 332. A computer-implemented method for use with a lung of a subject, the method comprising:
while an imaging device, disposed at a distal portion of a catheter, is disposed within an airway of the lung, receiving an input from the imaging device, the imaging device having a field of view;
referencing:
   a three-dimensional model of the airway, and
   a planned route through the model;
identifying, within the model, a viewing frustum that corresponds to the field of view of the imaging device; and
generating an output that includes:
   an output image derived from the input, and
   superimposed on the output image, an indication of a part of the planned route that appears within the viewing frustum.

Example 333. The method according to example 332, wherein the method is performed iteratively as the field of view changes.

Example 334. The method according to any one of examples 332-333, wherein the output is provided via a user interface to a user.

Example 335. The method according to any one of examples 332-334, wherein the input from the imaging device comprises a video feed, and wherein generating the output comprises generating the output image from the video feed.

Example 336. The method according to any one of examples 332-335, wherein:
the part of the planned route that appears within the viewing frustum corresponds to an upcoming part of the route to be followed by a distal portion of the catheter, and
generating the output comprises superimposing on the output image the upcoming part of the planned route.

Example 337. The method according to any one of examples 332-336, wherein identifying the viewing frustum includes simulating lighting conditions corresponding to actual lighting used by the imaging device.

Example 338. The method according to any one of examples 332-337, wherein:
the imaging device comprises a light source, and
generating the output image comprises generating the output image acquired during illumination of the airway by the light source.

Example 339. The method according to any one of examples 332-338, wherein superimposing on the output image the indication of the part of the planned route, comprises superimposing a series of dots on the output image.

Example 340. The method according to any one of examples 332-339, wherein superimposing on the output image the indication of the part of the planned route, comprises superimposing an arrow on the output image.

Example 341. The method according to any one of examples 332-340, wherein superimposing on the output image the indication of the part of the planned route, comprises superimposing on the output image a distinct marking/coloration of the airway that lies on the part of the planned route.

Example 342. The method according to any one of examples 332-341, wherein superimposing on the output image the indication of the part of the planned route, comprises superimposing on the output image a visual indication of the planned route.

Example 343. The method according to any one of examples 332-342, further comprising:
referencing an expected disposition, along the route, of a representation of the imaging device; and
responsively to the expected disposition and to the identified viewing frustum, updating the model to become more representative of the airway.

Example 344. The method according to example 343, wherein the method further comprises determining the expected disposition responsively to receiving a sensor output from a sensor configured to sense advancement of the catheter.

Example 345. The method according to any one of examples 332-344, further comprising:
referencing an expected disposition, along the route, of a representation of the imaging device; and
responsively to the expected disposition and to the identified viewing frustum, determining a refined disposition, along the route, of the representation of the imaging device, the refined disposition being more representative, than the expected disposition, of a true position of the imaging device within the airway.

Example 346. The method according to example 345, wherein determining the refined disposition comprises updating the model in a manner that refines the expected disposition into the refined disposition.

Example 347. The method according to example 345, wherein the method further comprises determining the expected disposition responsively to receiving a sensor output from a sensor configured to sense advancement of the catheter.

Example 348. The method according to any one of examples 332-347, wherein identifying the viewing frustum comprises:
generating a set of possible viewing frustums, the set of candidate viewing frustums based on an expected disposition of the imaging device with respect to the three-dimensional model, and
from the set of candidate viewing frustums, selecting the viewing frustum that most closely matches the field of view.

Example 349. The method according to example 348, wherein the expected disposition of the imaging device is based on an output from a sensor, and wherein the method further comprises identifying the expected disposition of the imaging device by reading the output of the sensor.

Example 350. The method according to any one of examples 332-349, further comprising advancing the imaging device along the planned route by manipulating a head of the catheter, the head being functionally coupled to the distal portion of the catheter via a wire.

Example 351. The method according to example 350, wherein manipulating the head of the catheter comprises rotating the head.

Example 352. The method according to example 350, wherein manipulating the head of the catheter comprises pulling on the wire, such that the wire changes a curvature of a distal portion of the catheter.

Example 353. The method according to any one of examples 332-352, wherein:
the method further comprises generating, from the viewing frustum, a derived image, and
identifying the viewing frustum comprises matching the derived image to the input from the imaging device.

Example 354. The method according to example 353, wherein:
identifying the viewing frustum comprises selecting the viewing frustum from a plurality of candidate viewing frustums within an expected range of the three-dimensional model, each of the candidate viewing frustums having a corresponding derived image, and
selecting the viewing frustum from the plurality of candidate viewing frustums comprises comparing, with the input from the imaging device, the derived image of each of the candidate viewing frustums.

Example 355. The method according to example 353, wherein generating the derived image comprises calculating light reflections from a virtual light source within the model in a manner that the derived image matches the input from the imaging device.

Example 356. The method according to any one of examples 332-355, wherein:
the imaging input is a three-dimensional image acquired in real time, and
comparing image inputs acquired iteratively by the imaging device comprises comparing image inputs in a manner that creates a real-time three-dimensional representation of the airway.

Example 357. A system, comprising:
a catheter comprising:
   a head, at a proximal portion of the catheter, and
   a tube, extending from the head toward a distal portion of the catheter;
a manipulator assembly configured to receive and engage the catheter in a manner that configures the manipulator assembly to manipulate the distal portion of the catheter through an airway of a subject; and
a control system configured to electronically operate the manipulator assembly, and comprising a data-processing system comprising means for carrying out the steps of Example 332.

Example 358. The system according to example 357, wherein the catheter is sterilized.

Example 359. The system according to any one of examples 357-358, further comprising an imaging device, positionable at the distal portion of the catheter.

Example 360. The system according to any one of examples 357-359, further comprising a sensor, configured to:
sense manipulation of the catheter; and
provide:
   an advancement output indicative of a sensed linear manipulation of the catheter; and
   a rotation output indicative of a sensed rotational manipulation of the catheter.

Example 361. The system according to any one of examples 357-360, wherein:
the catheter is a first catheter;
the manipulator assembly is a first manipulator assembly;
the system further comprises:
   a second catheter; and
   a second manipulator assembly;
the first manipulator assembly is configured to receive and engage the first catheter in a manner that configures the first manipulator assembly to manipulate a distal portion of the first catheter through the airway; and
the second manipulator assembly is configured to receive and engage the second catheter in a manner that configures the second manipulator assembly to manipulate a distal portion of the second catheter through the airway.

Example 362. The system according to example 361, further comprising:
a first sensor configured to sense the manipulation of the first catheter by the first manipulator assembly, and
a second sensor configured to sense the manipulation of the second catheter by the second manipulator assembly,
   wherein, for each of the first sensor and the second sensor, a sensor output reflective of the manipulation of the respective catheter is provided to the control system, and the control system is configured to use the sensor output to update the generated output.

Example 363. A computer-implemented method for use with an imaging device within an airway of a lung of a subject, the method comprising:
while the imaging device is disposed at a first site within the airway such that the imaging device has a first field of view, receiving a first input from the imaging device;
identifying, within a three-dimensional model of the airway, a first viewing frustum that corresponds to the first field of view;
while the imaging device is disposed at a second site having a second field of view, receiving a second input from the imaging device;
identifying, within the three-dimensional model, a second viewing frustum that corresponds to the second field of view; and
responsively to identifying the first viewing frustum and the second viewing frustum, adjusting at least a part of the three-dimensional model.

Example 364. The method according to example 363, wherein adjusting at least a part of the three-dimensional model comprises adjusting the part of the three-dimensional model between the first viewing frustum and the second viewing frustum.

Example 365. The method according to any one of examples 363-364, wherein adjusting the part of the three-dimensional model comprises scaling the three-dimensional model to match a difference in scale between the part of the model, and at least one of the first field of view and the second field of view.

Example 366. The method according to any one of examples 363-365, wherein adjusting the part of the three-dimensional model comprises rotating a depiction of the airway within the model.

Example 367. The method according to any one of examples 363-366, wherein the method is performed iteratively, the computer receiving iterative inputs from the imaging device as the imaging device moves along the airway.

Example 368. The method according to any one of examples 363-367, wherein a distance between the first field of view and the second field of view comprises a part of a planned route through the model, and wherein adjusting the part of the model comprises adjusting a part of the planned route through the model.

Example 369. The method according to example 368, wherein adjusting the part of the planned route comprises adjusting a distance between subsequent forks of the airway.

Example 370. The method according to example 368, wherein adjusting the part of the planned route comprises adjusting an angle between subsequent forks of the airway.

Example 371. The method according to any one of examples 363-370, further comprising:
referencing a planned route through the model; and
subsequently to adjusting at least the part of the three-dimensional model, and while the imaging device remains at the second site, generating an output that includes:
   an output image derived from the second input, and
   an indication of a part of the planned route that appears within the second viewing frustum.

Example 372. The method according to example 371, wherein the method further comprises providing a display of at least part of the planned route through a corresponding part of the three-dimensional model.

Example 373. A computer-implemented method for use with a lung of a subject, the method comprising:
while an imaging device, disposed at a distal portion of a catheter, is disposed in a true disposition within an airway of the lung, receiving an input from the imaging device, the imaging device having a field of view;
referencing:
   a three-dimensional model of the airway,
   a planned route through the model, and
   an expected disposition, along the route, of a representation of the imaging device;
identifying, within the model, a viewing frustum that corresponds to the field of view of the imaging device, and
responsively to the identified viewing frustum, determining a refined disposition, along the route, of the representation of the imaging device, the refined disposition being more representative, than the expected disposition, of the true disposition.

Example 374. The method according to example 373, wherein the true disposition comprises a true position and a true orientation, and wherein being disposed in the true disposition comprises being disposed in the true position and true orientation.

Example 375. The method according to any one of examples 373-374, wherein the refined disposition comprises a refined position and a refined orientation, and wherein determining the refined disposition comprises determining the refined position and refined orientation of the representation of the imaging device.

Example 376. The method according to any one of examples 373-375, wherein the expected disposition comprises an expected disposition and an expected orientation, and wherein referencing the expected disposition comprises referencing the expected disposition and expected orientation of the representation of the imaging device.

Example 377. The method according to any one of examples 373-376, wherein the method comprises referencing a portion of the three-dimensional model within a threshold distance of the expected disposition, and within the threshold distance, matching the viewing frustum to the field of view.

Example 378. The method according to any one of examples 373-377, wherein identifying the viewing frustum comprises identifying similar anatomical features in the received input and in a referenced portion of the three-dimensional model.

Example 379. The method according to any one of examples 373-378, wherein determining the refined disposition of the imaging device provides a refined indication of a disposition of the distal portion of the catheter.

Example 380. The method according to any one of examples 373-379, further comprising:
generating an output that includes:
   an input image derived from the input from the imaging device, and
   superimposed on the input image, an indication of a part of the planned route that appears within the viewing frustum.

Example 381. The method according to any one of examples 373-380, wherein:
the planned route is determined by an algorithm preoperatively; and
the method further comprises guiding a distal steering region of the catheter according to the planned route preoperatively determined by the algorithm.

Example 382. The method according to any one of examples 373-381, wherein:
the planned route is determined by an operator intraoperatively; and
the method further comprises guiding a distal steering region according to the planned route intraoperatively determined by the operator.

Example 383. The method according to any one of examples 373-382, further comprising:
receiving a position input from a sensor indicative of a linear and rotational position of the imaging device with respect to the airway; and
wherein determining the refined disposition of the representation of the imaging device comprises refining the linear and rotational disposition of the representation the imaging device with respect to the model.

Example 384. The method according to example 383, wherein receiving the position input from the sensor comprises receiving input from the sensor indicative of a linear and rotational disposition of the imaging device.

Example 385. The method according to example 383, wherein receiving the position input from the sensor comprises, using the sensor, measuring a distance traveled by, and rotation of, the imaging device.

Example 386. The method according to any one of examples 373-385, wherein the method further comprises calculating a quantitative difference between the refined disposition and the expected disposition.

Example 387. The method according to example 386, wherein the quantitative difference is provided in three dimensions, such that calculating the quantitative difference comprises calculating the quantitative difference in three dimensions.

Example 388. The method according to example 386, wherein the method further comprises outputting the quantitative difference as an adjustment to the three-dimensional model.

Example 389. The method according to any one of examples 373-388, wherein the method is performed iteratively as the catheter follows the planned route to a target, such that receiving the input from the imaging device comprises receiving a video feed.

Example 390. The method according to example 389, wherein in at least some iterations of the method, the expected disposition differs from the true disposition, such that determining the refined disposition requires an adjustment to the model.

Example 391. The method according to example 390, wherein the required adjustments improve accuracy of the model, such that the refined disposition approaches the true disposition as the catheter approaches the target.

Example 392. The method according to any one of examples 373-391, wherein the method further comprises determining the expected disposition of the representation of the imaging device based on receiving input from a sensor.

Example 393. The method according to example 392, wherein referencing the expected disposition comprises referencing the expected disposition derived by a controller with respect to sensor input indicative of a distance traveled along the planned route.

Example 394. The method according to example 393, wherein the method further comprises displaying an indication of the refined disposition by the controller.

Example 395. A system, comprising:
a catheter, comprising:
   a head, at a proximal portion of the catheter; and
   a tube, extending from the head toward a distal portion of the catheter;
an imaging device;
a manipulator structure comprising:
   a manipulator assembly configured to receive and engage the catheter in a manner that configures the manipulator assembly to manipulate the distal portion of the catheter through an airway of a subject, and
   a sensor, configured to:
      sense the manipulation of the catheter by the manipulator assembly, and
      provide a manipulation output indicative of the sensed manipulation of the catheter; and
a control system:
   configured to:
      electronically operate the manipulator structure,
      receive the manipulator output; and
   comprising a data-processing system comprising means for carrying out the steps of example 373, the control system determining the expected disposition responsively to the manipulation output.

Example 396. The system according to example 395, wherein the catheter is sterilized.

Example 397. The system according to any one of examples 395-396, wherein the sensor is configured to measure movement of the catheter by determining:
a length of the tube moving past a reference point on the manipulator assembly; and
rotation of the tube from an initial position.

Example 398. The system according to any one of examples 395-397, wherein the manipulation output comprises a measurement of a distance traveled by a distal end of the tube.

Example 399. The system according to any one of examples 395-398, wherein the manipulation output comprises a measurement of rotation of the tube as determined by determining a degree of rotation of the head.

Example 400. The system according to any one of examples 395-399, wherein the control system is configured to verify an expected disposition of the distal portion of the catheter in the airway.

Example 401. The system according to any one of examples 395-400, wherein the imaging device used for a distal part of the operative plan is an ultrasound probe.

Example 402. The system according to example 401, wherein the ultrasound probe is configured to produce planar images.

Example 403. The system according to example 402, wherein determination of the expected disposition is based on three-dimensional reconstruction of a plurality of stacked planar images acquired intraoperatively.

Example 404. The system according to example 402, wherein the system is configured to align/stack the planar images to produce a three-dimensional image intraoperatively.

Example 405. The system according to any one of examples 395-404, further comprising an imaging device, positionable at the distal portion of the catheter.

Example 406. The system according to example 405, wherein the imaging device is an ultrasound transceiver.

Example 407. The system according to example 405, wherein the imaging device is a camera.

Example 408. The system according to example 407, wherein the camera has a fiber optic light source.

Example 409. The system according to example 407, wherein the camera is configured to provide a video output of a position of a distal end of the catheter.

Example 410. The system according to any one of examples 395-409, wherein:
the catheter is a first catheter;
the system further comprises a second catheter;
the manipulator assembly is a first manipulator assembly;
the system further comprises a second manipulator assembly;
the sensor is a first sensor;
the manipulation output is a first manipulation output;
the system further comprises a second sensor;
the second manipulator assembly is configured to receive and engage the second catheter in a manner that configures the second manipulator assembly to manipulate a distal portion of the second catheter through the airway; and
the second sensor is configured to:
   sense the manipulation of the second catheter by the second manipulator assembly; and
   provide a second manipulation output indicative of the sensed manipulation of the second catheter.

Example 411. The system according to example 410, wherein:

the first catheter is configured to carry a tool;
the second catheter is configured to carry an imaging device;
the first manipulation output provides information on the expected disposition of the tool; and
the second manipulation output provides information on the expected disposition of the imaging device.

Example 412. The system according to example 411, wherein the control system is configured to:
record a tool site for the tool;
compare the first manipulation output with the tool site; and
provide a tool comparison output indicating the expected disposition of the tool relative to the tool site.

Example 413. The system according to example 411, wherein the control system is configured to:
record an imaging site for the imaging device;
compare the second manipulation output with the imaging site; and
provide an imaging comparison output indicating the expected disposition of the imaging device relative to the imaging site.

Example 414. A computer-implemented method for use with a lung of a subject, the method comprising:
while an imaging device, disposed at a distal portion of a catheter, is disposed in a true position within an airway of the lung, receiving an input from the imaging device;
referencing:
   a three-dimensional model of the airway,
   a planned route through the model, and
   an expected disposition, along the planned route, of a representation of the imaging device; and
determining a refined position, along the route, of the representation of the imaging device, the refined position being more representative, than the expected disposition, of the true position.

Example 415. A system for performing a bronchoscopic procedure on a lung of a subject, the system comprising:
a catheter, comprising:
   a head; and
   a tube, having a steering region at a distal portion thereof;
an extracorporeal manipulator structure that:
   comprises a manipulator assembly and a sensor; and
   is configured to receive the catheter such that:
      the manipulator assembly becomes engaged with the catheter; and
      the sensor is configured to:
         sense linear advancement of the catheter; and
         provide an advancement output indicative of the sensed linear advancement; and
a control system, electronically connectable with the manipulator structure, and comprising a data-processing system that comprises:
   a manipulator module, configured to instruct the manipulator assembly to linearly advance the catheter;
   a tracing module, configured to:
      reference a three-dimensional model of a lung of a subject, the model including a representation of an airway of the lung,
      receive the advancement output, and
      responsively to the advancement output, determine an expected disposition within the representation of the airway corresponding to a disposition of the distal portion of the catheter along the airway; and
   a display module, configured to provide a navigational guide responsively to the disposition determined by the tracing module.

Example 416. The system according to example 415, wherein the catheter is sterilized.

Example 417. The system according to any one of examples 415-416, wherein:
the system further comprises:
   an imaging device having a field of view, and being extendable through the catheter, and
   a model-image bridging module configured to, while (i) the distal portion of the tube is disposed within the airway and (ii) the imaging device is disposed at the distal portion of the tube:
   reference a planned route through the model,
   receive an imaging input from the imaging device,
   identify, within the model, a viewing frustum that corresponds to the field of view of the imaging device, and
   responsively to the expected disposition and the identified viewing frustum, refine the expected disposition into a refined disposition, and
the navigational guide comprises an output that includes:
   an output image derived from the imaging input, and
   superimposed on the output image, an indication of a part of the planned route that appears within the viewing frustum, positioning of the indication with respect to the output image being responsive to the refined disposition.

Example 418. The system according to any one of examples 415-417, wherein the advancement output comprises a measurement of a distance traveled by a distal end of the tube as determined by a length of the tube moving past a reference point on the manipulator assembly.

Example 419. The system according to any one of examples 415-418, wherein the advancement output is determined by mechanical contact of the sensor with the tube.

Example 420. The system according to any one of examples 415-419, wherein the sensor is an electromechanical sensor.

Example 421. The system according to any one of examples 415-420, wherein sensing of linear advancement is via optical observation of the tube.

Example 422. The system according to any one of examples 415-421, wherein the sensor is configured to record and output a length of tube passing the sensor.

Example 423. The system according to any one of examples 415-422, wherein the navigational guide comprises an indication of the planned route beyond the identified viewing frustum.

Example 424. The system according to any one of examples 415-423, wherein the navigational guide comprises an indication of a part of a planned route distal to the refined disposition.

Example 425. The system according to any one of examples 415-424, wherein the sensor is positioned in proximity to an entry point of the tube to the subject.

Example 426. The system according to any one of examples 415-425, wherein the sensor is configured to track and output rotation of the tube.

Example 427. The system according to any one of examples 415-426, wherein the control system is configured to use the advancement output to adjust a position of the steering region.

Example 428. The system according to any one of examples 415-427, wherein the sensor is configured to generate output indicative of a position of the steering region in three-dimensional space.

Example 429. The system according to any one of examples 415-428, wherein a tool is disposed at a distal end of the catheter, and the system is configured to verify a position of the tool.

Example 430. The system according to any one of examples 415-429, wherein:
the manipulator structure further comprises a rotation sensor configured to:
   sense rotation of the catheter, and
   provide a rotation output indicative of the sensed rotation, and
the tracing module is configured to:
   receive the rotation output, and
   determine the expected disposition responsively to the advancement output and the rotation output.

Example 431. The system according to example 430, wherein the navigational guide includes a rotation indication that is indicative of the rotation output.

Example 432. The system according to any one of examples 415-431, wherein:
the manipulator structure further comprises a bending sensor configured to:
   sense bending of the catheter, and
   provide a bending output indicative of the sensed bending, and
the tracing module is configured to:
   receive the bending output, and
   determine the expected disposition responsively to the advancement output and the bending output.

Example 433. The system according to example 432, wherein the navigational guide includes a bending indication that is indicative of the bending output.

Example 434. The system according to any one of examples 415-433, wherein the manipulator assembly comprises a steering manipulator and an advancement manipulator, and the manipulator assembly becomes engaged with the catheter by the steering manipulator receiving the head and the advancement manipulator receiving the tube of the catheter.

Example 435. The system according to example 434, wherein the sensor is further configured to sense rotation of the tube and provide a rotational output indicative of the sensed rotation.

Example 436. The system according to example 435, wherein the steering manipulator is configured to sense bending of the tube and to provide a bending output indicative of the sensed bending.

Example 437. The system according to example 436, wherein the tracing module is configured to:
receive the advancement output, the rotational output, and the bending output, and
determine the disposition responsively to the advancement output, the rotational output, and the bending output.

Example 438. The system according to example 437, wherein the manipulator structure further comprises a force sensor configured to sense force exerted on the steering region, and to provide a force output indicative thereof.

Example 439. The system according to example 438, wherein the control system further comprises means to maintain a bending state of the steering region by automatically responding to the force output.

Example 440. The system according to any one of examples 415-439, further comprising:
an imaging device, positionable at the distal portion of the catheter; and
a data-processing system comprising means for carrying out the steps of Example 332.

Example 441. The system according to example 440, wherein the control system comprises the data-processing system, configured to electronically operate the extracorporeal manipulator structure.

Example 442. The system according to any one of examples 415-441, further comprising:
an imaging device, positionable at the distal portion of the catheter; and
a data-processing system comprising means for carrying out the steps of Example 373.

Example 443. The system according to example 442, wherein the control system comprises the data-processing system, configured to electronically operate the manipulator structure.

Example 444. The system according to any one of examples 415-443, wherein the catheter further comprises a first wire, and a second wire, each of the first wire and the second wire extending from the steering region proximally along the tube, and the head further comprises:
a stem,
a first plunger, operatively coupled to the steering region by being attached to the first wire, and mounted on the stem to be slidable linearly along the stem, and
a second plunger, operatively coupled to the steering region by being attached to the second wire, and mounted on the stem to be slidable linearly along the stem independently of the first plunger.

Example 445. The system according to example 444, wherein the steering manipulator is configured to bend the steering region by sliding the first plunger.

Example 446. The system according to example 444, wherein the steering manipulator is configured to straighten the steering region by sliding the second plunger.

Example 447. A system for use with a subject, comprising:
a catheter, comprising:
   a head at a proximal region of the catheter, and
   a tube, having a distal portion configured to be advanced into the subject via a body orifice of the subject, and having a steering region at the distal portion; and
a robot, comprising a manipulator assembly, the manipulator assembly:
   comprising a steering manipulator and an advancement manipulator, and
   configured to be loaded with the catheter such that:
      the steering manipulator receives the head in a manner that operatively couples the steering manipulator to the steering region such that a curvature of the steering region is adjustable by the steering manipulator manipulating the head, and
      the advancement manipulator receives the tube such that operation of the advancement manipulator feeds the tube through the advancement manipulator in a manner that (i) pulls the head and the steering manipulator distally toward the advancement manipulator and the body orifice, and (ii) pushes the tube distally through the body orifice into the subject.

Example 448. A method for use with a real or simulated subject, the subject having a real or simulated body orifice, the method comprising:
into an advancement manipulator of a manipulator assembly, loading a tube of a catheter, the tube having a steering region at a distal portion of the tube, and the catheter having a head at a proximal region of the catheter;
loading the head into a steering manipulator of the manipulator assembly, the steering manipulator being configured to adjust a curvature of the steering region by manipulating the head; and
operating the advancement manipulator to feed the tube through the advancement manipulator in a manner that (i) pulls the head and the steering manipulator distally toward the advancement manipulator and the body orifice, and (ii) pushes the tube distally through the body orifice into the subject.

Example 449. A method for use with a real or simulated subject, the subject having a real or simulated body orifice, the method comprising:
positioning an advancement manipulator of a manipulator assembly at the body orifice; and
while (i) a tube of a catheter is disposed through the advancement manipulator, and (ii) a head of the catheter is disposed within a steering manipulator of the advancement assembly, operating the advancement manipulator to feed the tube through the advancement manipulator in a manner that (i) pulls the head and the steering manipulator distally toward the advancement manipulator and the body orifice, and (ii) pushes the tube distally through the body orifice into the subject.

The present invention is not limited to the examples that have been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A system for use with a lung of a subject, the system comprising:
a catheter, comprising:
a head at a proximal region of the catheter, and
a tube, having a distal portion configured to be advanced into the lung via a mouth of the subject, and having a steering region at the distal portion; and
a robot, comprising a manipulator structure that defines an advancement path and comprises a manipulator assembly, the manipulator assembly comprising a steering manipulator and an advancement manipulator, and configured to be loaded with the catheter such that:
the steering manipulator receives the head in a manner that operatively couples the steering manipulator to the steering region such that a curvature of the steering region is adjustable by the steering manipulator manipulating the head, and
the advancement manipulator receives a part of the tube such that operation of the advancement manipulator feeds the tube through the advancement manipulator in a manner that (i) pulls the head and the steering manipulator distally along the advancement path toward the advancement manipulator and the mouth, and (ii) pushes at least the part of the tube distally through the mouth toward the lung.

2.-116. (canceled)

117. The system according to claim 1, wherein the steering manipulator is configured to, while remaining operatively coupled to the steering region, rotate the steering region by rotating the head.

118. The system according to claim 1, wherein the manipulator assembly is configured such that, while remaining loaded with the catheter, feeding of the tube through the advancement manipulator (i) pulls the head and the steering manipulator distally along the advancement path by a distance, and (ii) pushes the part of the tube distally by the same distance.

119. The system according to claim 1, wherein:
the manipulator assembly is configured such that the advancement manipulator is positionable distally from the steering manipulator to define a separation, between the steering manipulator and the advancement manipulator, of at least 40 cm, and
the manipulator assembly is configured such that the steering manipulator is pullable, by the advancement manipulator feeding the tube, to reduce the separation to less than 20 cm.

120. The system according to claim 1, wherein the steering manipulator is biased to retreat proximally along the advancement path.

121. The system according to claim 120, wherein the biasing maintains a straightness of the tube between the steering manipulator and the advancement manipulator.

122. The system according to claim 1, wherein the catheter further comprises:
a first wire, and a second wire, each of the first wire and the second wire extending from the steering region proximally along the tube; and
wherein the head further comprises:
a stem,
a first plunger, operatively coupled to the steering region by being attached to the first wire, and mounted on the stem to be slidable linearly along the stem, and
a second plunger, operatively coupled to the steering region by being attached to the second wire, and mounted on the stem to be slidable linearly along the stem independently of the first plunger.

123. The system according to claim 122, wherein the steering manipulator is configured to receive the head in a manner that operatively couples the steering manipulator to the first plunger and the second plunger in a manner that configures the steering manipulator to control sliding of the first plunger and the second plunger linearly along the stem.

124. The system according to claim 122, wherein application of a sliding force to either plunger slides the plunger in a first direction along the stem in a manner that adjusts a curvature of the steering region by applying tension to the respective wire, and releasing the sliding force allows the respective wire to relax, by the wire responsively pulling the plunger in a reverse direction along the stem.

125. The system according to claim 122, wherein application of a force to the head adjusts a curvature of the steering region by applying tension to one of the first wire or the second wire, and concurrent tensioning of both wires adjusts a stiffness of the steering region such that the steering region maintains a specific curvature.

126. The system according to claim 122, wherein the first wire is operatively coupled to the steering region in a force-multiplication arrangement configured to increase a mechanical advantage of the first wire on the steering region.

127. The system according to claim 1, further comprising:
an imaging device, positionable at the distal portion of the catheter; and
a data-processing system comprising means for carrying out the steps of:
(i) while the imaging device, at the distal portion of the catheter, is disposed within an airway of the lung, receiving an input from the imaging device, the imaging device having a field of view;
(ii) referencing:
a three-dimensional model of the airway, and
a planned route through the model;
(iii) identifying, within the model, a viewing frustum that corresponds to the field of view of the imaging device; and
(iv) generating an output that includes:
an output image derived from the input, and
superimposed on the output image, an indication of a part of the planned route that appears within the viewing frustum.

128. The system according to claim 127, wherein the robot comprises a robotic control system that comprises the data-processing system, and that is configured to electronically operate the manipulator structure.

129. The system according to claim 1, wherein the robot further comprises a robotic control system, configured to electronically control the manipulator structure.

130. The system according to claim 129, wherein the manipulator assembly is a first manipulator assembly, and the manipulator structure comprises a second manipulator assembly, the robotic control system configured to electronically coordinate control of the first manipulator assembly and the second manipulator assembly.

131. The system according to claim 1, wherein the manipulator structure comprises a track that defines at least part of the advancement path, the steering manipulator being slidably mounted on the track such that the steering manipulator slides along the track responsively to feeding of the tube through the advancement manipulator.

132. The system according to claim 1, wherein the catheter is configured such that the steering region becomes limp responsively to the head being disengaged from the steering manipulator.

133. The system according to claim 1, wherein:
the head comprises:
a stem,
a first plunger, operatively coupled to the steering region via a first wire, and mounted on the stem to be slidable linearly along the stem, and
a second plunger, operatively coupled to the steering region via a second wire, and mounted on the stem to be slidable linearly along the stem independently of the first plunger; and
the steering manipulator comprises:
a first control unit, comprising:
a first actuator,
a first spring, and
a first cradle, coupled to the first actuator via the first spring, and configured to receive the first plunger, and
a second control unit, comprising:
a second actuator,
a second spring, and
a second cradle, coupled to the second actuator via the second spring, and configured to receive the second plunger, and
the steering manipulator is configured to manipulate the steering region by:
actuating the first actuator to, via the first spring, slide the first plunger linearly along the stem, and
actuating the second actuator to, via the second spring, slide the second plunger linearly along the stem.

134. The system according to claim 1, further comprising a sensor, disposed distally to the advancement manipulator, and configured to sense forward and rotational movement of the tube with respect to the advancement manipulator.

135. The system according to claim 1, wherein:
the manipulator assembly is a first manipulator assembly,
the catheter is a first catheter, and
the system further comprises:
a second catheter, and
a second manipulator assembly, defining a second advancement path, such that the robot is configured to control the first catheter and the second catheter independently and in parallel with each other.
